SEQUENCE LISTING (69 sequences)

SEQ ID NO: 1 - DNA, 957 bp, Homo sapiens
  CDS (1)..(954)
atgatggtggatcccaatggcaatgaatccagtgctacatacttcatcctaataggcctccctggtttagaagaggctcagttctggttggccttcccattgtgctccctctaccttattgctgtgctaggtaacttgacaatcatctacattgtgcggactgagcacagcctgcatgagcccatgtatatatttctttgcatgctttcaggcattgacatcctcatctccacctcatccatgcccaaaatgctggccatcttctggttcaattccactaccatccagtttgatgcttgtctgctacagatgtttgccatccactccttatctggcatggaatccacagtgctgctggccatggcttttgaccgctatgtggccatctgtcacccactgcgccatgccacagtacttacgttgcctcgtgtcaccaaaattggtgtggctgctgtggtgcggggggctgcactgatggcaccccttcctgtcttcatcaagcagctgcccttctgccgctccaatatcctttcccattcctactgcctacaccaagatgtcatgaagctggcctgtgatgatatccgggtcaatgtcgtctatggccttatcgtcatcatctccgccattggcctggactcacttctcatctccttctcatatctgcttattcttaagactgtgttgggcttgacacgtgaagcccaggccaaggcatttggcacttgcgtctctcatgtgtgtgctgtgttcatattctatgtacctttcattggattgtccatggtgcatcgctttagcaagcggcgtgactctccgctgcccgtcatcttggccaatatctatctgctggttcctcctgtgctcaacccaattgtctatggagtgaagacaaaggagattcgacagcgcatccttcgacttttccatgtggccacacacgcttcagagccctag

SEQ ID NO: 2 - protein, 318 aa, Homo sapiens
MMVDPNGNESSATYFILIGLPGLEEAQFWLAFPLCSLYLIAVLGNLTIIYIVRTEHSLHEPMYIFLCMLSGIDILISTSSMPKMLAIFWFNSTTIQFDACLLQMFAIHSLSGMESTVLLAMAFDRYVAICHPLRHATVLTLPRVTKIGVAAVVRGAALMAPLPVFIKQLPFCRSNILSHSYCLHQDVMKLACDDIRVNVVYGLIVIISAIGLDSLLISFSYLLILKTVLGLTREAQAKAFGTCVSHVCAVFIFYVPFIGLSMVHRFSKRRDSPLPVILANIYLLVPPVLNPIVYGVKTKEIRQRILRLFHVATHASEP

SEQ ID NO: 3 - DNA, 1381 bp, Homo sapiens
ccacgcgtccgctctgccctgaatccaggatagaccaggacaacaagatgagtggctaactgtaggatggtgtccatctgtgctctaggggaggagtagcatcaaaggagaagcaagaactgagaactgtttggggcactgaagaagtaggactaaggaagagttagggggttagtacaaatctgaggcctggttttctggaaagagaccagagactgaccttattgcatgtcatacaacatgcttgcttagagacccctaatttattttcttctcttactctttctgaggaagcatgagccacaccctcagttagttttgtataatcttaggcttgatgagaatataatcttagtcttgaaggctttaaaggggaagaaatagctgtctgtgttagtggtgtgtcagtcagcaggagaacctgctaggggtggaaggaggagggtaggagtatagcctagaccatgagtagataccccgctccaccttgaaagtctcctactggacctcttatgatggagttaatacctcctgtttcctctattccagattgttttcagtttccagaaggcaaaactgacatctcccaggagtccaagtaggagattagggcctcccgtccctatctactcagtgctagccttggctaagagagaggaaattcctgcctagaggggaaaatctgcaggacttcgttaccactttcactttggcagaggaaggaggtcagggatggaaggggaagtgagtctagaaattaaaacatagaattctgtctacaggtggtggagagcctttctgaaagtgcttctgggttgaggctgtcacctagattttatattagagtttaagtgttccaaaaaattaagaagcaggaagtagaaaagagaacaatttcagaagcagacgaaaggaacagtaataggaagatctagcaaggatgtggtggggcagtttcagtgtgagatgccatggacaggaaaatggcagcatatgtgtgtgtgtgtgtgtgtgtgtgtgtccatgagacagagagacataaataactaaataaaaaggcatatcacaaagaggggctcctgcttcagcttgagtcctggatgcaaagacatgtggactgggatcctagcaacctatctgcagccaaggacatgacgttagacgccccaagaaaaggaaaattggtcaaacataggaagagcactcaagtgccagctacagtgaatgacaaatacccaccacaagcacaagctctacattcacaaaaacttggaaaacacaagttcatagactgggcaaccctgagtagtggagagatcaccagccatgtttcaggttgtaccctctacctgcctggtgctggtcacagttcagcttcttc

SEQ ID NO: 4 - DNA, 2034 bp, Homo sapiens
gtgtcagtgatcaaacttcttttccattcagagtcctctgattcagattttaatgttaacattttggaagacagtattcagaaaaaaaatttccttaataaaaatacaactcagatccttcaaatatgaaactggttggggaatctccattttttcaatattattttcttctttgttttcttgctacatataattattaataccctgactaggttgtggttggagggttattacttttcattttaccatgcagtccaaatctaaactgcttctactgatggtttacagcattctgagataagaatggtacatctagagaacatttgccaaaggcctaagcacggcaaaggaaaataaacacagaatataataaaatgagataatctagcttaaaactataacttcctcttcagaactcccaaccacattggatctcagaaaaatactgtcttcaaaatgacttctacagagaagaaataatttttcctctggacactagcacttaaggggaagattggaagtaaagccttgaaaagagtacatttacctacgttaatgaaagttgacacactgttctgagagttttcacagcatatggaccctgtttttcctatttaattttcttatcaaccctttaattaggcaaagatattattagtaccctcattgtagccatgggaaaattgatgttcagtggggatcagtgaattaaatggggtcatacaagtataaaaattaaaaaaaaaagacttcatgcccaatctcatatgatgtggaagaactgttagagagaccaacagggtagtgggttagagatttccagagtcttacattttctagaggaggtatttaatttcttctcactctctccagtgttgtatttaggaatttcctggcaacagaactcatggctttaatcccactagctattgcttattgtcctggtccaattgccaattacctgtgtcttggaagaagtgatttctaggttcaccattatggaagattcttattcagaaagtctgcatagggcttatagcaagttatttatttttaaaagttccataggtgattctgataggcagtgaggttagggagccaccagttatgatgggaagtatggaatggcaggtcttgaagataacattggccttttgagtgtgactcgtagctggaaagtgagggaatcttcaggaccatgctttatttggggctttgtgcagtatggaacagggactttgagaccaggaaagcaatctgacttaggcatgggaatcaggcatttttgcttctgaggggctattaccaagggttaataggtttcatcttcaacaggatatgacaacagtgttaaccaagaaactcaaattacaaatactaaaacatgtgatcatatatgtggtaagtttcattttctttttcaatcctcaggttccctgatatggattcctataacatgctttcatccccttttgtaatggatatcatatttggaaatgcctatttaatacttgtatttgctgctggactgtaagcccatgagggcactgtttattattgaatgtcatctctgttcatcattgactgctctttgctcatcattgaatcccccagcaaagtgcctagaacataatagtgcttatgcttgacaccggttatttttcatcaaacctgattccttctgtcctgaacacatagccaggcaattttccagccttctttgagttgggtattattaaattctggccattacttccaatgtgagtggaagtgacatgtgcaatttctatacctggctcataaaaccctcccatgtgcagcctttcatgttgacattaaatgtgacttgggaagctatgtgttacacagagtaaatcaccagaagcctggatttctgaaaaaactgtgcagagccaaacctctgtcatttgcaactcccacttgtatttgtacgaggcagttggataagtgaaaaataaagtactattgtgtcaagtcaaaaaaaaaaaaaaaaaaaaaaaaaaaaaaaaaaaa

SEQ ID NO: 5 - DNA, 80 bp, Homo sapiens
gatccaccatcatgaagaagctgaactgtgaccagcaccaggcaggtagaggctcaaccgtatggaaggaatgtgtgacc

SEQ ID NO: 6 - DNA, 20 bp, Homo sapiens
actgagcacagcctgcatga

SEQ ID NO: 7 - DNA, 25 bp, Homo sapiens
tctgtagcagacaagcatcaaactg

SEQ ID NO: 8 - protein, 311 aa, Mus musculus
MWPNSSDAPFLLTGFLGLEMIHHWISIPFFVIYFSIIVGNGTLLFIIWSDHSLHEPMYYFLAVLASMDLGMTLTTMPTVLGVLVLNQREIVHGACFIQSYFIHSLAIVESGVLLAMSYDRFVAICTPLHYNSILTNSRVMKMALGALLRGFVSIVPPIMPLFWFPYCHSHVLSHAFCLHQDVMKLACADITFNLIYPVVLVALTFFLDALIIIFSYVLILKKVMGIASGEERKKSLNTCVSHISCVLVFYITVIGLTFIHRFGKNAPHVVHITMSYVYFLFPPFMNPIIYSIKTKQIQRSILRLLSKHSRT

SEQ ID NO: 9 - protein, 307 aa, Mus musculus
MWSNISAAPFLLTGFPGLEAAHHWISIPFFAIYISVLLGNGTLLYLIKDDHNLHEPMYYFLAMLAGTDLTVTLTTMPTVMAVLWVNHREIRHGACFLQAYIIHSLSIVESGVLLAMSYDRFVAICTPLHYNSILTNSRVIAIGLGVVLRGFLSLVPPILPLFWFSYCRSHVLSHAFCLHQDVMKLACADITFNRIYPVVLVALTFFLDALIIVFSYVLILKTVMGIASGEERAKALNTCVSHISCVLVFYITVIGLTFIHRFGKNAPHVVHITMSYVYFLFPPFMNPIIYSIKTKQIQRSVLHLLSV

SEQ ID NO: 10 - protein, 312 aa, HUMAN
MWPNITAAPFLLTGFPGLEAAHHWISIPFFAVYVCILLGNGMLLYLIKHDHSLHEPMYYFLTMLAGTDLMVTLTTMPTVMGILWVNHREISSVGCFLQAYFIHSLSVVESGSLLAMAYDRFIAIRNPLRYASIFTNTRVIALGVGVFLRGFVSILPVILRLFSFSYCKSHVITRAFCLHQEIMRLACADITFNRLYPVILISLTIFLDSLIILFSYILILNTVIGIASGEEQTKALNTCVSHFCAVLIFYIPLAGLSIIHRYGRNAPPISHAVMANVYLFVPPILNPVIYSIKTKQIQYGIIRLLSKHRFSR

SEQ ID NO: 11 - protein, 319 aa, CHICKEN
MYPRNSSQAQPFLLAGLPGMAQFHHWVFLPFGLMYLVAVLGNGTILLVVRVHRQLHQPMYYFLLMLATTDLGLTLSTLPTVLRVFWLGAMEISFPACLIQMFCIHVFSFMESSVLLAMAFDRYVAICCPLRYSSILTGARVAQIGLGIICRCTLSLLPLICLLTWLPFCRSHVLSHPYCLHQDIIRLACTDATLNSLYGLILVLVAILDFVLIALSYIMIFRTVLGITSKEEQTKALNTCVSHFCAVLIFYIPLAGLSIIHRYGRNAPPISHAVMANVYLFVPPILNPVLYSMKSKAICKGLLRLLCQRAAWPGHAQNC

SEQ ID NO: 12 - protein, 320 aa, Rattus norvegicus
MSSCNFTHATFMLIGIPGLEEAHFWFGFPLLSMYAVALFGNCIVVFIVRTERSLHAPMYLFLCMLAAIDLALSTSTMPKILALFWFDSREITFDACLAQMFFIHALSAIESTILLAMAFDRYVAICHPLRHAAVLNNTVTVQIGMVALVRGSLFFFPLPLLIKRLAFCHSNVLSHSYCVHQDVMKLAYTDTLPNVVYGLTAILLVMGVDVMFISLSYFLIIRAVLQLPSKSERAKAFGTCVSHIGVVLAFYVPLIGLSVVHRFGNSLDPIVHVLMGDVYLLLPPVINPIIYGAKTKQIRTRVLAMFKISCDKDIEAGGNT

SEQ ID NO: 13 - protein, 321 aa, Mus musculus
MNSKASMLGTNFTIIHPTVFILLGIPGLEQYHTWLSIPFCLMYIAAVLGNGALILVVLSERTLHEPMYVFLSMLAGTDILLSTTTVPKTLAIFWFHAGEIPFDACIAQMFFIHVAFVAESGILLAMAFDRYVAICTPLRYSAVLTPMAIGKMTLAIWGRSIGTIFPIIFLLKRLSYCRTNVIPHSYCEHIGVARLACADITVNIWYGFSVPMASVLVDVALIGISYTLILQAVFRLPSQDARHKALNTCGSHIGVILLFFIPSFFTFLTHRFGKNIPHHVHILLANLYVLVPPMLNPIIYGAKTKQIRDSMTRMLSVVWKS

SEQ ID NO: 14 - protein, 326 aa, Mus musculus
MKVASSFHNDTNPQDVWYVLIGIPGLEDLHSWIAIPICSMYIVAVIGNVLLIFLIVTERSLHEPMYFFLSMLALADLLLSTATAPKMLAIFWFHSRGISFGSCVSQMFFIHFIFVAESAILLAMAFDRYVAICYPLRYTTILTSSVIGKIGTAAVVRSFLICFPFIFLVYRLLYCGKHIIPHSYCEHMGIARLACDNITVNIIYGLTMALLSTGLDILLIIISYTMILRTVFQIPSWAARYKALNTCGSHICVILLFYTPAFFSFFAHRFGGKTVPRHIHILVANLYVVVPPMLNPIIYGVKTKQIQDRVVFLFSSVSTCQHDSRC

SEQ ID NO: 15 - protein, 318 aa, Mus musculus
  MISC_FEATURE (286)..(286): wherein “X” is unknown.
MSPGNSSWIHPSSFLLLGIPGLEELQFWLGLPFGTVYLIAVLGNVIILFVIYLEHSLHQPMFYLLAILAVTDLGLSTATVPRALGIFWFGFHKIAFRDCVAQMFFIHLFTGIETFMLVAMAFDRYIAICNPLRYNTILTNRTICIIVGVGLFKNFILVFPLIFLILRLSFCGHNIIPHTYCEHMGIARLACVSIKVNVLFGLILISMILLDVVLSALSYAKILHAVFKLPSWEARLKALNTCGSHVCVILAFFTPAFFSFLTHRFGHNIPRYIHILLANLYVIIPXALNPIIYGVRTKQIQDRAVTILCNEVGQLADD

SEQ ID NO: 16 - protein, 316 aa, Mus musculus
MIKFNGSVFMPSVLTLVGIPGLESVQCWIGIPFCVMYIIAMIGNSLILVIIKSEKSLHIPMYIFLAILAVTDIALSTCILPKMLGIFWFHMPQISFDACLLQMELIHSFQATESGILLAMALDRYVAICNPLRHATIFSPQLTTCLGAGALLRSLITTFPLILLIKFCLKYFRTTIISHSYCEHMAIVKLAAQDIRINKICGLLVAFAILGFDIVFITFSYVRIFITVFQLPQKEARFKAFNTCIAHICVFLQFYLLAFFSFFTHRFGAHIPPYVHILLSDLYLLVPPFLNPIVYGIKTKQIRDQVLKMFFSKKPL

SEQ ID NO: 17 - protein, 27 aa, Homo sapiens
MMVDPNGNESSATYFILIGLPGLEEAQ

SEQ ID NO: 18 - protein, 11 aa, Homo sapiens
RTEHSLHEPMY

SEQ ID NO: 19 - protein, 14 aa, Homo sapiens
NSTTIQFDACLLQM

SEQ ID NO: 20 - protein, 16 aa, Homo sapiens
HPLRHATVLTLPRVTK

SEQ ID NO: 21 - protein, 30 aa, Homo sapiens
KQLPFCRSNILSHSYCLHQDVMKLACDDIR

SEQ ID NO: 22 - protein, 14 aa, Homo sapiens
KTVLGLTREAQAKA

SEQ ID NO: 23 - protein, 10 aa, Homo sapiens
HRFSKRRDSP

SEQ ID NO: 24 - protein, 22 aa, Homo sapiens
KTKEIRQRILRLFHVATHASEP

SEQ ID NO: 25 - DNA, 22 bp, Homo sapiens
cctgtgctcaacccaattgtct

SEQ ID NO: 26 - DNA, 22 bp, Homo sapiens
actgacacctagggctctgaag

SEQ ID NO: 27 - DNA, 17 bp, Homo sapiens
agccgagccacatcgct

SEQ ID NO: 28 - DNA, 19 bp, Homo sapiens
gtgaccaggcgcccaatac

SEQ ID NO: 29 - DNA, 28 bp, Homo sapiens
caaatccgttgactccgaccttcacctt

SEQ ID NO: 30 - DNA, 39 bp, Homo sapiens
cccaagcttgcaccatgatggtggatcccaatggcattg

SEQ ID NO: 31 - DNA, 33 bp, Homo sapiens
gaagatctctagggctctgaagcgtgtgtggcc

SEQ ID NO: 32 - DNA, 59 bp, Homo sapiens
gaagatctctacttgtcgtcgtcgtccttgtagtccatgggctctgaagcgtgtgtggc

SEQ ID NO: 33 - protein, 13 aa, Homo sapiens
MVHRFSKRRDSPL

SEQ ID NO: 34 - protein, 14 aa, Homo sapiens
VRTEHSLHEPMYIF

SEQ ID NO: 35 - protein, 14 aa, Homo sapiens
FLCMLSGIDILIST

SEQ ID NO: 36 - protein, 14 aa, Homo sapiens
AIHSLSGMESTVLL

SEQ ID NO: 37 - protein, 14 aa, Homo sapiens
HRFSKRRDSPLPVI

SEQ ID NO: 38 - protein, 14 aa, Homo sapiens
VDPNGNESSATYFI

SEQ ID NO: 39 - protein, 14 aa, Homo sapiens
IAVLGNLTIIYIVR

SEQ ID NO: 40 - protein, 14 aa, Homo sapiens
AIFWFNSTTIQFDA

SEQ ID NO: 41 - protein, 16 aa, Homo sapiens
MVDPNGNESSATYFIL

SEQ ID NO: 42 - protein, 16 aa, Homo sapiens
LIGLPGLEEAQFWLAF

SEQ ID NO: 43 - protein, 16 aa, Homo sapiens
IHSLSGMESTVLLAMA

SEQ ID NO: 44 - protein, 16 aa, Homo sapiens
QAKAFGTCVSHVCAVF

SEQ ID NO: 45 - protein, 27 aa, Homo sapiens
HSLSGMESTVLLAMAFDRYVAICHPLR

SEQ ID NO: 46 - DNA, 99 bp, Artificial Sequence
  Synthesized Random Oligonucleotide.
cgaagcgtaagggcccagccggccnnknnknnknnknnknnknnknnknnknnknnknnknnknnknnknnknnknnknnknnkccgggtccgggcggc

SEQ ID NO: 47 - DNA, 95 bp, Artificial Sequence
  Synthesized Random Oligonucleotide.
aaaaggaaaaaagcggccgcvnnvnnvnnvnnvnnvnnvnnvnnvnnvnnvnnvnnvnnvnnvnnvnnvnnvnnvnnvnngccgcccggacccgg

SEQ ID NO: 48 - protein, 5 aa, Artificial Sequence
  Consensus sequence.
PGPGG

SEQ ID NO: 49 - DNA, 38 bp, Homo sapiens
gcagcagcggccgccagttctggttggccttcccattg

SEQ ID NO: 50 - DNA, 36 bp, Homo sapiens
gcagcagtcgacgggctctgaagcgtgtgtggccac

SEQ ID NO: 51 - DNA, 39 bp, Homo sapiens
gcagcagcggccgcatgatggtggatcccaatggcaatg

SEQ ID NO: 52 - DNA, 37 bp, Homo sapiens
gcagcagtcgaccttcactccatagacaattgggttg

SEQ ID NO: 53 - protein, 15 aa, Artificial Sequence
  Synthesized Polypeptide.
GDFWYEACESSCAFW

SEQ ID NO: 54 - protein, 15 aa, Artificial Sequence
  Synthesized Polypeptide.
CLRSGTGCAFQLYRF

SEQ ID NO: 55 - protein, 15 aa, Artificial Sequence
  Synthesized Polypeptide.
FAGQIIWYDALDTLM

SEQ ID NO: 56 - protein, 15 aa, Artificial Sequence
  Synthesized Polypeptide.
LIFFDARDCCFNEQL

SEQ ID NO: 57 - protein, 15 aa, Artificial Sequence
  Synthesized Polypeptide.
LEWGSDVFYDVYDCC

SEQ ID NO: 58 - protein, 15 aa, Artificial Sequence
  Synthesized Polypeptide.
RIVPNGYFNVHGRSL

SEQ ID NO: 59 - protein, 15 aa, Artificial Sequence
  Synthesized Polypeptide.
WERSSAGCADQQYRC

SEQ ID NO: 60 - protein, 15 aa, Artificial Sequence
  Synthesized Polypeptide.
YFSDGESFFEPGDCC

SEQ ID NO: 61 - DNA, 23 bp, Homo sapiens
cattgactgctctttgctcatca

SEQ ID NO: 62 - DNA, 23 bp, Homo sapiens
aataaccggtgtcaagcataagc

SEQ ID NO: 63 - DNA, 33 bp, Homo sapiens
tgaatcccccagcaaagtgcctagaacataata

SEQ ID NO: 64 - protein, 19 aa, Homo sapiens
KEIRQRILRLFHVATHASE

SEQ ID NO: 65 - DNA, 25 bp, Artificial Sequence
  Synthesized Oligonucleotide.
ggucuaggcuauacuccuacccucc

SEQ ID NO: 66 - DNA, 25 bp, Artificial Sequence
  Synthesized Oligonucleotide.
ggacaccauccuacaguuagccacu

SEQ ID NO: 67 - DNA, 25 bp, Artificial Sequence
  Synthesized Oligonucleotide.
ccuccuuccucugccaaagugaaag

SEQ ID NO: 68 - DNA, 25 bp, Artificial Sequence
  Synthesized Oligonucleotide.
ccuguccauggcaucucacacugaa

SEQ ID NO: 69 - DNA, 25 bp, Artificial Sequence
  Synthesized Oligonucleotide.
ccaggccucagauuuguacuaaccc

(19) United States
(12) Patent Application Publication
(10) Pub. No.: US 2004/0121330 A1
(43) Pub. Date: Jun. 24, 2004

(54) Novel human G-protein coupled receptor, HGPRBMY4, and methods of use thereof

(76) Inventors: John N. Feder, Belle Mead, NJ (US); Gabriel A. Mintier, Hightstown, NJ (US); Chandra S. Ramanathan, Wallingford, CT (US); Donald R. Hawken, Lawrenceville, NJ (US); Angela Cacace, Clinton, CT (US); Lauren E. Barber, Higganum, CT (US); Michael G. Kornacker, Princeton, NJ (US); Rolf-Peter Ryseck, Ewing, NJ (US); Kelly L. Bennett, Skillman, NJ (US); Thomas C. Nelson, Lawrenceville, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/323,412

(22) Filed: Dec. 18, 2002

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention describes a newly discovered human G-protein coupled receptor and its encoding polynucleotide. Also described are expression vectors, host cells, agonists, antagonists, antisense molecules, and antibodies associated with the polynucleotide and polypeptide of the present invention. Methods for treating, diagnosing, preventing, and screening for neurological, cardiovascular, and prostate-, colon-, breast-, or lung-related conditions or disorders are described.

This application claims benefit to non-provisional application U.S. Ser. No. 09/966,459, filed Sep. 26, 2001; which claims benefit to provisional application U.S. Serial. No. 60/235,833, filed Sep. 27, 2000; to provisional application U.S. Serial. No. 60/261,776, filed Jan. 16, 2001; to provisional application U.S. Serial. No. 60/305,351, filed Jul. 13, 2001; and to provisional application U.S. Serial. No. 60/313,202, filed Aug. 17, 2001.

### FIELD OF THE INVENTION

The present invention relates to the fields of pharmacogenomics, diagnostics, and patient therapy. More specifically, the present invention relates to methods of diagnosing and treating diseases involving the Human G-Protein Coupled Receptor, HGPRBMY4.

### BACKGROUND OF THE INVENTION

It is well established that many medically significant biological processes are mediated by proteins participating in signal transduction pathways that involve G-proteins and second messengers, for example, cAMP (Lefkowitz, Nature, 351:353-354 (1991)). Herein these proteins are referred to as proteins participating in pathways with G-proteins or PPG proteins. Some examples of these proteins include the GPC receptors, such as those for adrenergic agents and dopamine (Kobilka, B. K., et al., PNAS, 84:46-50 (1987); Kobilka, B. K., et al., Science, 238:650-656 (1987); Bunzow, J. R., et al., Nature, 336:783-787 (1988)), G-proteins themselves, effector proteins, for example, phospholipase C, adenylate cyclase, and phosphodiesterase, and actuator proteins, for example, protein kinase A and protein kinase C (Simon, M. I., et al., Science, 252:802-8 (1991)).

For example, in one form of signal transduction, the effect of hormone binding is activation of an enzyme, adenylate cyclase, inside the cell. Enzyme activation by hormones is dependent on the presence of the nucleotide GTP, and GTP also influences hormone binding. A G-protein connects the hormone receptors to activated by hormone receptors. The GTP-carrying form then binds to an activated. adenylate cyclase. Hydrolysis of GTP to GDP, catalyzed by the G-protein itself, returns the G-protein to its basal, inactive form. Thus, the G-protein serves a dual role, as an intermediate that relays the signal from receptor to effector, and as a clock that controls the duration of the signal.

G-protein coupled receptors (GPCRs) are one of the largest receptor superfamilies known. The structure of GPCRs consists of seven conserved hydrophobic stretches of about 20 to 30 amino acids or transmembrane alpha helical domains that are connected by at least eight divergent extracellular or cytoplasmic hydrophilic loops. Most G-protein coupled receptors have single conserved cysteine residues in each of the first two extracellular loops which form disulfide bonds that are believed to stabilize functional protein structure. The 7 transmembrane (TM) regions are designated as TM1, TM2, TM3, TM4, TM5, TM6, and TM7. TM3 has been implicated in signal transduction. The N-terminus is always extracellular and C-terminus is intracellular. Phosphorylation and lipidation (palmitylation or famesylation) of cysteine residues can influence signal transduction of some G-protein coupled receptors. Most G-protein coupled receptors contain potential phosphorylation sites within the third cytoplasmic loop or the carboxyl terminus. For several G-protein coupled receptors, such as the β-adrenoreceptor, phosphorylation by protein kinase A or specific receptor kinases mediates receptor desensitization.

For some receptors, the ligand binding sites of G-protein coupled receptors are believed to comprise a hydrophilic socket formed by several G-protein coupled receptors transmembrane domains, where the socket is surrounded by hydrophobic residues of the G-protein coupled receptors. The hydrophilic side of each G-protein coupled receptor transmembrane helix is postulated to face inward and form the polar ligand-binding site. TM3 has been implicated in several G-protein coupled receptors as having a ligand-binding site, such as including the TM3 aspartate residue. Additionally, TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc. Rev., 10:317-331(1989)). Different G-protein β-subunits preferentially stimulate particular effectors to modulate various biological functions in a cell. Phosphorylation of cytoplasmic residues of G-protein coupled receptors have been identified as an important mechanism for the regulation of G-protein coupling of some G-protein coupled receptors. G-protein coupled receptors are found in numerous sites within a mammalian host. GPCRs are involved in signal transduction. The signal is received at the extracellular N-terminus side. The signal can be an endogenous ligand, a chemical moiety, or light. This signal is then transduced through the membrane to the cytosolic side where a heterotrimeric protein G-protein is activated which in turn elicits a response (F. Horn et al., Recept. and Chann., 5: 305-314 (1998)). Ligands, agonists and antagonists for these GPCRs useful for therapeutic purposes.

The membrane protein gene superfamily of G-protein coupled receptors has been characterized as having seven putative transmembrane domains. The domains are believed to represent transmembrane alpha-helices connected by extracellular or cytoplasmic loops. G-protein coupled receptors include a wide range of biologically active receptors, such as hormone, viral, growth factor and neuroreceptors. The G-protein family of coupled receptors includes dopamine receptors, which bind to neuroleptic drugs, used for treating psychotic and neurological disorders. Other examples of members of this family include calcitonin, adrenergic, endothelin, cAMP, adenosine, muscarinic, acetylcholine, serotonin, histamine, thrombin, kinin, follicle stimulating hormone, opsins, endothelial differentiation gene-1 receptor, rhodopsins, odorant, cytomegalovirus receptors, etc. These receptors are biologically important and malfunction of these receptors results in diseases such as Alzheimer's, Parkinson's, diabetes, dwarfism, color blindness, retinal pigmentosa and asthma. GPCRs are also involved in depression, schizophrenia, insomnia, hypertension, anxiety, stress, renal failure and in several other cardiovascular, metabolic, neuronal, oncology-related and immune disorders (F. Horn and G. Vriend, J. Mol. Med., 76: 464-468 (1998)). They have also been shown to play a role in HIV infection (Y. Feng et al., Science, 272: 872-877 (1996)).

The fate of a cell in multicellular organisms often requires choosing between life and death. This process of cell suicide, known as programmed cell death or example, in development of an embryo, during the course of an immunological response, or in the demise of cancerous cells after drug treatment, among others. The final outcome of cell survival versus apoptosis is dependent on the balance of two counteracting events, the onset and speed of caspase cascade activation (essentially a protease chain reaction), and the delivery of antiapoptotic factors which block the caspase activity (Aggarwal B. B. Biochem. Pharmacol. 60, 1033-1039, (2000); Thomberry, N. A. and Lazebnik, Y. Science 281, 1312-1316, (1998)).

The production of antiapoptotic proteins is controlled by the transcriptional factor complex NFkB. For example, exposure of cells to the protein tumor necrosis factor (TNF) can signal both cell death and survival, an event playing a major role in the regulation of immunological and inflammatory responses (Ghosh, S., May, M. J., Kopp, E. B. Annu. Rev. Immunol. 16, 225-260, (1998); Silverman, N. and Maniatis, T., Genes & Dev. 15, 2321-2342, (2001); Baud, V. and Karin, M., Trends Cell Biol. 11, 372-377, (2001)). The anti-apoptotic activity of NFkB is also crucial to oncogenesis and to chemo- and radio-resistance in cancer (Baldwin, A. S., J. Clin. Invest. 107, 241-246, (2001)).

Nuclear Factor kappa B (NFkB), is composed of dimeric complexes of p50 (NFkB1) or p52 (NFkB2) usually associated with members of the Rel family (p65, c-Rel, Rel B) which have potent transactivation domains. Different combinations of NFkB/Rel proteins bind distinct kappa B sites to regulate the transcription of different genes. Early work involving NFkB suggested its expression was limited to specific cell types, particularly in stimulating the transcription of genes encoding kappa immunoglobulins in B lymphocytes. However, it has been discovered that NFkB is, in fact, present and inducible in many, if not all, cell types and that it acts as an intracellular messenger capable of playing a broad role in gene regulation as a mediator of inducible signal transduction. Specifically, it has been demonstrated that NFkB plays a central role in regulation of intercellular signals in many cell types. For example, NFkB has been shown to positively regulate the human beta-interferon (beta-IFN) gene in many, if not all, cell types. Moreover, NFkB has also been shown to serve the important function of acting as an intracellular transducer of external influences.

The transcription factor NFkBis sequestered in an inactive form in the cytoplasm as a complex with its inhibitor, IkB, the most prominent member of this class being IkB alpha. A number of factors are known to serve the role of stimulators of NFkBactivity, such as, for example, TNF. After TNF exposure, the inhibitor is phosphorylated and proteolytically removed, releasing NFkBinto the nucleus and allowing its transcriptional activity. Numerous genes are upregulated by this transcription factor, among them IkB alpha. The newly synthezised IkB alpha protein inhibits NFKB, effectively shutting down further transcriptional activation of its downstream effectors. However, as mentioned above, the IkB alpha protein can only inhibit NFKB in the absence of IrB alpha stimuli, such as TNF stimulation, for example. Other agents that are known to stimulate NFKB release, and thus NFkB activity, are bacterial lipopolysaccharide, extracellular polypeptides, chemical agents, such as phorbol esters, which stimulate intracellular phosphokinases, inflammatory cytokines, IL-1, oxidative and fluid mechanical stresses, and ionizing radiation (Basu, S., Rosenzweig, K, R., Youmell, M., Price, B, D, Biochem. Biophys. Res. Commun., 247(1):79-83, (1998)). Therefore, as a general rule, the stronger the insulting stimulus, the stronger the resulting NFkB activation, and the higher the level of IkB alpha transcription. As a consequence, measuring the level of WB alpha RNA can be used as a marker for antiapoptotic events, and indirectly, for the onset and strength of pro-apoptotic events.

It has been shown that the IkB promoter is driven by NFkB and by an NFkB-independent arsenite/heat stress response (Nucleic Acids Res. 1994; 22:3787, J. Clin. Invest. 1997; 99:2423). In addition, the E-selectin promoter has been shown to be activated by NFkB, but that elevated levels of cAMP can inhibit TNF-alpha stimulation of E-selectin expression on endothelial cells (J. Biol. Chem. 1996; 271: 20828, J. Biol. Chem. 1994; 269: 19193). Likewise, LPS stimulation of TNF-alpha expression, a promoter that is also driven by NFkB, has been shown to be inhibited by elevated cAMP in RAW246.7 and THP-1 cells, (J. Biol. Chem. 1996; 271: 20828, J. Biol. Chem. 1996; 273:31427). While the signaling pathway responsible for driving the NFkB-independent arsenite/heat induced stress response has not yet been defined, stress induced by arsenite in PC12 cell has been shown to stimulate ATF/CREB family members (cAMP responsive element-binding proteins) to drive Gadd153 expression (J. Biochem. 1999; 339: 135).

### SUMMARY OF THE INVENTION

The present invention provides a novel human member of the GPCR family (HGPRBMY4). Based on sequence homology, the protein HGPRBMY4 is a candidate GPCR. This protein sequence has been predicted to contain seven transmembrane domains, which is a characteristic structural feature of GPCRs. This orphan GPCR is expressed highly in prostate, colon, breast and lung with moderate expression in the heart.

The present invention provides an isolated HGPRBMY4 polynucleotide as depicted in SEQ ID NO: 1 (CDS: 1 to 2211).

The present invention also provides the HGPRBMY4 polypeptide (MW: 35.4 Kd), encoded by the polynucleotide of SEQ ID NO: 1 and having the amino acid sequence of SEQ ID NO: 2, or a functional or biologically active portion thereof.

The present invention further provides compositions comprising the HGPRBMY4 polynucleotide sequence, or a fragment thereof, or the encoded HGPRBMY4 polypeptide, or a fragment or portion thereof. Also provided by the present invention are pharmaceutical compositions comprising at least one HGPRBMY4 polypeptide, or a functional portion thereof, wherein the compositions further comprise a pharmaceutically acceptable carrier, excipient, or diluent.

The present invention provides a novel isolated and substantially purified polynucleotide that encodes the GPCR homologue. In a particular aspect, the polynucleotide comprises the nucleotide sequence of SEQ ID NO: 1. The present invention also provides a polynucleotide sequence comprising the complement of SEQ ID NO: 1, or variants thereof. In addition, the present invention features polynucleotide sequences, which hybridize under moderately stringent or high stringency conditions to the polynucleotide sequence of SEQ ID NO: 1.

The present invention further provides a nucleic acid sequence encoding the HGPRBMY4 polypeptide and an antisense of the nucleic acid sequence, as well as oligonucleotides, fragments, or portions of the nucleic acid molecule or antisense molecule. Also provided are expression vectors and host cells comprising polynucleotides that encode the HGPRBMY4 polypeptide.

The present invention provides methods for producing a polypeptide comprising the amino acid sequence depicted in SEQ ID NO: 2, or a fragment thereof, comprising the steps of a) cultivating a host cell containing an expression vector containing at least a functional fragment of the polynucleotide sequence encoding the HGPRBMY4 homologue according to this invention under conditions suitable for the expression of the polynucleotide; and b) recovering the polypeptide from the host cell.

Also provided are antibodies, and binding fragments thereof, which bind specifically to the HGPRBMY4 polypeptide, or an epitope thereof, for use as therapeutics and diagnostic agents.

The present invention also provides methods for screening for agents which modulate HGPRBMY4 polypeptide, as well as modulators, for example, agonists and antagonists, particularly those that are obtained from the screening methods described.

Also provided by the present invention is a substantially purified antagonist or inhibitor of the polypeptide of SEQ ID NO: 2. In this regard, and by way of example, a purified antibody that binds to a polypeptide comprising the amino acid sequence of SEQ ID NO: 2 is provided.

Substantially purified agonists of the G-protein coupled receptor polypeptide of SEQ ID NO: 2 are further provided.

The present invention provides HGPRBMY4 nucleic acid sequences, polypeptide, peptides and antibodies for use in the diagnosis and/or screening of disorders or diseases associated with expression of the polynucleotide and its encoded polypeptide as described herein.

The present invention provides kits for screening and diagnosis of disorders associated with aberrant or uncontrolled cellular development and with the expression of the polynucleotide and its encoded polypeptide as described herein.

The present invention further provides methods for the treatment or prevention of cancers, immune disorders, neurological, or prostate-, colon-, lung-, breast-, and cardiovascular-related disorders involving administering, to an individual in need of treatment or prevention, an effective amount of a purified antagonist of the HGPRBMY4 polypeptide. Due to its elevated levels of expression in specific tissues, the novel GPCR protein of the present invention is particularly useful in treating or preventing prostate-, colon-, lung-, breast-, and cardiovascular-related disorders, conditions, or diseases.

The present invention also provides a method for detecting a polynucleotide that encodes the HGPRBMY4 polypeptide in a biological sample comprising the steps of: a) hybridizing the complement of the polynucleotide sequence encoding SEQ ID NO: 2 to a nucleic acid material of a biological sample, thereby forming a hybridization complex; and b) detecting the hybridization complex, wherein the presence of the complex correlates with the presence of a polynucleotide encoding the HGPRBMY4 polypeptide in the biological sample. The nucleic acid material can be further amplified by the polymerase chain reaction prior to hybridization.

Further objects, features, and advantages of the present invention will be better understood upon a reading of the detailed description of the invention when considered in connection with the accompanying figures or drawings.

One aspect of the instant invention comprises methods and compositions to detect and diagnose alterations in the HGPRBMY4 sequence in tissues and cells as they relate to ligand response.

The present invention further provides compositions for diagnosing prostate-, colon-, lung-, breast-, and/or cardiovascular-related disorders and response to HGPRBMY4 therapy in humans. In accordance with the invention, the compositions detect an alteration of the normal or wild type HGPRBMY4 sequence or its expression product in a patient sample of cells or tissue.

Another embodiment provides diagnostic probes for diseases and a patient's response to therapy. The probe sequence comprises the HGPRBMY4 locus polymorphism. The probes can be constructed of nucleic acids or amino acids.

The invention further relates to a method for preventing, treating, or ameliorating a medical condition with the polypeptide provided as SEQ ID NO:2, in addition to, its encoding nucleic acid, or a modulator thereof, wherein the medical condition is a reproductive disorder; a male reproductive disorder; a prostate disorder; prostate cancer; proliferative condition of the prostate; cardiovascular disorder; heart disorder; pulmonary disorder; lung disorder; lung cancer; proliferative condition of the lung; gastrointestinal disorder; a colon disorder; colon cancer; female reproductive disorder; ovarian cancer; placental disorder; proliferative condition of the ovary; melanoma; vascular disorders; umbilical cord disorder; disorders associated with aberrant E-selectin expression or activity; disorders associated with aberrant NFkB expression or activity; disorders associated with aberrant IkBalpha expression or activity; an inflammatory disorder; an inflammatory disorder associated with abberant NFkB regulation or regulation of the NFkB pathway; and a proliferative disorder associated with abberant NFKB regulation or regulation of the NFkB pathway.

The invention further relates to a method of diagnosing a pathological condition or a susceptibility to a pathological condition in a subject comprising the steps of (a) determining the presence or amount of expression of the polypeptide of SEQ ID NO:2 in a biological sample; (b) and diagnosing a pathological condition or a susceptibility to a pathological condition based on the presence or amount of expression of the polypeptide relative to a control, wherein said condition is a member of the group consisting of a reproductive disorder; a male reproductive disorder; a prostate disorder; prostate cancer; proliferative condition of the prostate; cardiovascular disorder; heart disorder; pulmonary disorder; lung disorder; lung cancer; proliferative condition of the lung; gastrointestinal disorder; a colon disorder; colon cancer; female reproductive disorder; ovarian cancer; placental disorder; proliferative condition of the ovary; melanoma; vascular disorders; umbilical cord disorder; disorders associated with aberrant E-selectin expression or activity; disorders associated with aberrant NFkB expression or activity; disorders associated with aberrant IkBalpha expression or activity; an inflammatory disorder; an inflammatory disorder associated with abberant NFkB regulation or regulation of the NFkB pathway; and a proliferative disorder associated with abberant NFkB regulation or regulation of the NFkB pathway.

The invention relates to a method of preventing, treating, or ameliorating an inflammatory or immune-related disease or disorder comprising inhibiting E-selectin expression by administering to a mammal in need thereof, HGPRBMY4 polypeptide of SEQ ID NO: 2, homologue, or functional fragment thereof, in an amount effective to inhibit E-selectin expression.

The invention relates to a method of inhibiting activation of NFkB-dependent gene expression associated with the inhibition of E-selectin expression, comprising administering to a mammal in need thereof an amount of HGPRBMY4 polypeptide of SEQ ID NO: 2, or homologue thereof, effective to inhibit E-selectin expression, thereby inhibiting activation of NFkB-dependent gene expression.

The invention relates to a method of inhibiting E-selectin expression, comprising administering to a mammal in need thereof, an amount of HGPRBMY4 polypeptide of SEQ ID NO: 2, homologue, or fragment thereof, effective to inhibit E-selectin expression.

The invention relates to a method of treating, preventing, or ameliorating a disease, disorder, or condition, comprising administering the G-protein coupled receptor polynucieotide of SEQ ID NO:1 or polypeptide, homologue, modulator, or fragment thereof in an amount effective to treat, prevent or ameliorate the disease, disorder or condition, further comprising inhibiting E-selectin, wherein inhibition of E-selectin results in one or more of the following: (I) inhibition of E-selectin activity; (ii) inhibition of phosphorylation of IκB; (iii) inhibition of NFkB-dependent gene expression; or (iv) increase of cAMP.

A further embodiment provides antibodies that recognize and bind to the HGPRBMY4 protein. Such antibodies can be either polyclonal or monoclonal. Antibodies that bind to the HGPRBMY4 protein can be utilized in a variety of diagnostic and prognostic formats and therapeutic methods.

Another embodiment relates to diagnostic kits for the determination of the nucleotide sequence of human HGPRBMY4 alleles. The kits are based on amplification-based assays, nucleic acid probe assays, protein nucleic acid probe assays, antibody assays or any combination thereof.

Methods for detecting genetic predisposition, susceptibility and response to therapy related to the prostate, colon, lung, breast and heart are also provided. In accordance with the invention, the method comprises isolating a human sample, for example, blood or tissue from adults, children, embryos or fetuses, and detecting at least one alteration in the wild-type HGPRBMY4 sequence or its expression product from the sample, wherein the alterations are indicative of genetic predisposition, susceptibility or altered response to therapy related to the prostate, colon, lung, breast, and heart.

In addition, methods for making determinations as to which drug to administer, dosages, duration of treatment and the like are provided.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are CHO cells.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of CRE response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are HEK cells.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, and further wherein said cells express the polypeptide at either low, moderate, or high levels.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said candidate compound is a small molecule, a peptide, or an antisense molecule.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said candidate compound is a small molecule, a peptide, or an antisense molecule, wherein said candidate compound is an agonist or antagonist.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said candidate compound is a small molecule, a peptide, or an antisense molecule.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said candidate compound is a small molecule, a peptide, or an antisense molecule, wherein said candidate compound is an agonist or antagonist.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said cells express beta lactamase at low, moderate, or high levels.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2682, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said cells express beta lactamase at low, moderate, or high levels.

### BRIEF DESCRIPTION OF THE FIGURES

FIG. 1 shows the full length nucleotide sequence of cDNA clone HGPRBMY4, a human G-protein coupled receptor (SEQ ID NO: 1).

FIG. 2 shows the amino acid sequence (SEQ ID NO: 2) from the conceptual translation of the full length HGPRBMY4 cDNA sequence.

FIG. 3 shows the 5′ untranslated sequence of the orphan receptor, HGPRBMY4 (SEQ ID NO: 3).

FIG. 4 shows the 3′ untranslated sequence of the orphan receptor, HGPRBMY4 (SEQ ID NO: 4).

FIG. 5 shows the predicted transmembrane region of the HGPRBMY4 protein where the predicted transmembranes, bold-faced and underlined, correspond to the peaks with scores above 750.

FIGS. 6A-6B show the multiple sequence alignment of the translated sequence of the orphan G-protein coupled receptor, HGPRBMY4, where the GCG pileup program was used to generate the alignment with other G-protein coupled receptor sequences. The blackened areas represent identical amino acids in more than half of the listed sequences and the grey highlighted areas represent similar amino acids. As shown in FIGS. 6A-6B, the sequences are aligned according to their amino acids, where: HGPRBMY4 (SEQ ID NO: 2) is the translated full length HGPRBMY4 cDNA; Q9WVN4 (SEQ ID NO: 8) represents the mouse form of MOR 5′ Beta1; Q9WVN5 (SEQ ID NO: 9) is the mouse form of MOR 5′ Beta2; Q9Y5P1 (SEQ ID NO: 10) is the human form of HOR 5′ Beta3; Q9YH55 (SEQ ID NO: 11) is the chicken form of an olfactory receptor-like protein; O88628 (SEQ ID NO: 12) represents the rat form of olfactory GPCR RA1C; Q9WU89 (SEQ ID NO: 13) is the mouse form of odorant receptor S18; Q9WVD9 (SEQ ID NO: 14) is the mouse form of MOR 3′ Beta 1; Q9WU93 (SEQ ID NO: 15) is the mouse form of odorant receptor S46; and Q9WVD7 (SEQ ID NO: 16) is the mouse form of MOR 3′ Beta3.

FIG. 7 shows the expression profiling of the novel human orphan GPCR, HGPRBMY4, as described in Example 3.

FIG. 8 shows the expression profiling of the novel human orphan GPCR, HGPRBMY4, as described in Example 4 and Table I.

FIG. 9 shows the FACS profile of an untransfected CHO NFAT-CRE cell line.

FIG. 10 shows that the overexpression of HGPRBMY4 constitutively couples through the NFAT/CRE response element.

FIG. 11 shows the FACS profile of an untransfected CHO NFAT-G alpha 15 cell line.

FIG. 12 shows that the overexpression of HGPRBMY4 constitutively couples through the NFAT response element via the promiscuous G protein, G alpha 15.

FIG. 13 shows that expressed HGPRBMY4 localizes to the cell surface.

FIG. 14 shows that representative transfected CHO-NFAT/CRE cell lines with intermediate and high beta lactamase expression levels useful in screens to identify HGPRBMY4 agonists and antagonists.

FIG. 15 shows an expanded expression profile of the novel G-protein coupled receptor, HGPRBMY4. The figure illustrates the relative expression level of HGPRBMY4 amongst various mRNA normal tissue sources. As shown, the HGPRBMY4 polypeptide was expressed predominantly in the prostate, heart, and testis. Expression of HGPRBMY4 was also significantly expressed in the placenta, cerebral blood vessel and the umbilical cord. Expression data was obtained by measuring the steady state HGPRBMY4 mRNA levels by quantitative PCR using the PCR primer pair provided as SEQ ID NOs: 61 and 62, and Taqman™ probe (SEQ ID NO: 63) as described in Example 5 herein.

FIG. 16 shows an expanded expression profile of the novel human G-protein coupled receptor, HGPRBMY4, of the present invention. The figure illustrates the relative expression level of HGPRBMY4 amonst various mRNA tissue sources isolated from normal and tumor prostate tissues. As shown, the HGPRBMY4 polypeptide was expressed in the prostate tissues and no other tumor type evidenced altered expression.

FIG. 17 shows an expanded expression profile of HGPRBMY4. The figure illustrates the relative expression level of HGPRBMY4 amongst various mRNA tissue sources isolated from prostate tumors.

FIG. 18 shows an expanded expression profile of HGPRBMY4 in cell lines of breast origin.

FIG. 19 shows an expanded expression profile of HGPRBMY4 in cell lines of colon origin. The figure illustrates steady state RNA levels for HGPRBMY4.

FIG. 20 shows an expanded expression profile of HGPRBMY4 in cell lines of lung origin.

FIG. 21 shows relative expression of HGPRBMY4 in OCLP3, where total RNA from ovary and SHP-77 from lung carcinoma have the highest expression. Other tissues having high to moderate expression include the following: LS 174T (colon), A375 (melanoma), total RNA from breast and fetal lung, LNCAP prostate, NCI-N87.

### DETAILED DESCRIPTION OF THE INVENTION

The present invention provides a novel isolated polynucleotide and encoded polypeptide, the expression of which is high in prostate-, colon-, lung-, breast-, and cardiovascular-related tissues. This novel polypeptide is termed herein HGPRBMY4, an acronym for “Human G-Protein coupled Receptor BMY4.” HGPRBMY4 is also referred to as GPCR9.

In particular, the present invention provides a newly discovered G-protein coupled receptor protein, which can be involved in cellular growth properties in the prostate, colon, lung, breast, and heart based on its abundance in those specific tissues. The present invention also relates to newly identified polynucleotides, polypeptides encoded by such polynucleotides, the use of such polynucleotides and polypeptides, as well as the production of such polynucleotides and polypeptides. More particularly, the polypeptides of the present invention are human seven transmembrane receptors. In addition, the invention also relates to inhibiting the action of such polypeptides. A further embodiment of the invention relates to the HGPRBMY4 polypeptide and its involvement in the NFkB signaling pathway through modulation of E-selectin, either directly or indirectly.

### Definitions

The HGPRBMY4 polypeptide (or protein) refers to the amino acid sequence of substantially purified HGPRBMY4, which can be obtained from any species, preferably mammalian, and more preferably, human, and from a variety of sources, including natural, synthetic, semi-synthetic, or recombinant. Functional fragments of the HGPRBMY4 polypeptide are also embraced by the present invention.

An “agonist” refers to a molecule which, when bound to the HGPRBMY4 polypeptide, or a functional fragment thereof, increases or prolongs the duration of the effect of the HGPRBMY4 polypeptide. Agonists can include proteins, nucleic acids, carbohydrates, or any other molecules that bind to and modulate the effect of the HGPRBMY4 polypeptide. An antagonist refers to a molecule which, when bound to the HGPRBMY4 polypeptide, or a functional fragment thereof, decreases the amount or duration of the biological or immunological activity of the HGPRBMY4 polypeptide. “Antagonists” can include proteins, nucleic acids, carbohydrates, antibodies, or any other molecules that decrease or reduce the effect of the HGPRBMY4 polypeptide.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

“Nucleic acid sequence,” as used herein, refers to an oligonucleotide, nucleotide, or polynucleotide, and fragments or portions thereof, and to DNA or RNA of genomic or synthetic origin which can be single- or double-stranded, and represent the sense or anti-sense strand. By way of non-limiting example, fragments include nucleic acid sequences that are greater than 20-60 nucleotides in length, and preferably include fragments that are at least 70-100 nucleotides, or which are at least 1000 nucleotides or greater in length.

Similarly, “amino acid sequence” as used herein refers to an oligopeptide, peptide, polypeptide, or protein sequence, and fragments or portions thereof, and to naturally occurring or synthetic molecules. Amino acid sequence fragments are typically from about 5 to about 30, preferably from about 5 to about 15 amino acids in length and retain the biological activity or function of the HGPRBMY4 polypeptide.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule. In addition, the terms HGPRBMY4 polypeptide and HGPRBMY4 protein are used interchangeably herein to refer to the encoded product of the HGPRBMY4 nucleic acid sequence of the present invention.

A “variant” of the HGPRBMY4 polypeptide refers to an amino acid sequence that is altered by one or more amino acids. The variant can have “conservative” changes, wherein a substituted amino acid has similar structural or chemical properties, for example, replacement of leucine with isoleucine. More rarely, a variant can have “non-conservative” changes, for example, replacement of a glycine with a tryptophan. Minor variations can also include amino acid deletions or insertions, or both. Guidance in determining which amino acid residues can be substituted, inserted, or deleted without abolishing functional biological or immunological activity can be found using computer programs well known in the art, for example, DNASTAR software.

An “allele” or “allelic sequence” is an alternative form of the HGPRBMY4 nucleic acid sequence. Alleles can result from at least one mutation in the nucleic acid sequence and can yield altered mRNAs or polypeptides whose structure or function may or may not be altered. Any given gene, whether natural or recombinant, can have none, one, or many allelic forms. Common mutational changes, which give rise to alleles, are generally ascribed to natural deletions, additions, or substitutions of nucleotides. Each of these types of changes can occur alone, or in combination with the others, one or more times in a given sequence.

“Altered” nucleic acid sequences encoding the HGPRBMY4 polypeptide include nucleic acid sequences containing deletions, insertions and/or substitutions of different nucleotides resulting in a polynucleotide that encodes the same or a functionally equivalent HGPRBMY4 polypeptide. Altered nucleic acid sequences can further include polymorphisms of the polynucleotide encoding the HGPRBMY4 polypeptide; such polymorphisms may or may not be readily detectable using a particular oligonucleotide probe. The encoded protein can also contain deletions, insertions, or substitutions of amino acid residues, which produce a silent change and result in a functionally equivalent HGPRBMY4 protein. Deliberate amino acid substitutions can be made on the basis of similarity in polarity, charge, solubility, hydrophobicity, hydrophilicity, and/or the amphipathic nature of the residues, as long as the biological activity of the HGPRBMY4 protein is retained. For example, negatively charged amino acids can include aspartic acid and glutamic acid; positively charged amino acids can include lysine and arginine; and amino acids with uncharged polar head groups having similar hydrophilicity values can include leucine, isoleucine, and valine; glycine and alanine; asparagine and glutamine; serine and threonine; and phenylalanine and tyrosine.

“Peptide nucleic acid” (PNA) refers to an antisense molecule or anti-gene agent which comprises an oligonucleotide (“oligo”) linked via an amide bond, similar to the peptide backbone of amino acid residues. PNAs typically comprise oligos of at least 5 nucleotides linked to amino acid residues. PNAs may or may not terminate in positively charged amino acid residues to enhance binding affinities to DNA. Such amino acids include, for example, lysine and arginine among others. These small molecules stop transcript elongation by binding to their complementary strand of nucleic acid (P. E. Nielsen et al., 1993, Anticancer Drug Des., 8:53-63). PNA can be pegylated to extend their lifespan in the cell where they preferentially bind to complementary single stranded DNA and RNA.

“Oligonucleotides” or “oligomers” refer to a nucleic acid sequence, preferably comprising contiguous nucleotides, of at least about 6 nucleotides to about 60 nucleotides, preferably at least about 8 to 10 nucleotides in length, more preferably at least about 12 nucleotides in length for example, about 15 to 35 nucleotides, or about 15 to 25 nucleotides, or about 20 to 35 nucleotides, which can be typically used in PCR amplification assays, hybridization assays, or in microarrays. It will be understood that the term oligonucleotide is substantially equivalent to the terms primer, probe, or amplimer, as commonly defined in the art. It will also be appreciated by those skilled in the pertinent art that a longer oligonucleotide probe, or mixtures of probes, such as, degenerate probes, can be used to detect longer, or more complex, nucleic acid sequences, for example, genomic DNA. In such cases, the probe can comprise at least 20-200 nucleotides, preferably, at least 30-100 nucleotides, more preferably, 50-100 nucleotides.

“Amplification” refers to the production of additional copies of a nucleic acid sequence and is generally carried out using polymerase chain reaction (PCR) technologies, which are well known and practiced in the art (see, D. W. Dieffenbach and G. S. Dveksler, 1995, PCR Primer, a Laboratory Manual, Cold Spring Harbor Press, Plainview, N.Y.).

“Microarray” is an array of distinct polynucleotides or oligonucleotides synthesized on a substrate, such as paper, nylon, or other type of membrane; filter; chip; glass slide; or any other type of suitable solid support.

The term “antisense” refers to nucleotide sequences, and compositions containing nucleic acid sequences, which are complementary to a specific DNA or RNA sequence. The term “antisense strand” is used in reference to a nucleic acid strand that is complementary to the “sense” strand. Antisense (i.e., complementary) nucleic acid molecules include PNA and can be produced by any method, including synthesis or transcription. Antisense oligonucleotides may be single or double stranded. Double stranded RNA's may be designed based upon the teachings of Paddison et al., Proc. Nat. Acad. Sci., 99:1443-1448 (2002); and International Publication Nos. WO 01/29058, and WO 99/32619; which are hereby incorporated herein by reference. Once introduced into a cell, the complementary nucleotides combine with natural sequences produced by the cell to form duplexes, which block either transcription or translation. The designation “negative” is sometimes used in reference to the antisense strand, and “positive” is sometimes used in reference to the sense strand.

The term “consensus” refers to the sequence that reflects the most common choice of base or amino acid at each position among a series of related DNA, RNA or protein sequences. Areas of particularly good agreement often represent conserved functional domains.

A “deletion” refers to a change in either nucleotide or amino acid sequence and results in the absence of one or more nucleotides or amino acid residues. By contrast, an insertion (also termed “addition”) refers to a change in a nucleotide or amino acid sequence that results in the addition of one or more nucleotides or amino acid residues, as compared with the naturally occurring molecule. A substitution refers to the replacement of one or more nucleotides or amino acids by different nucleotides or amino acids.

A “derivative” nucleic acid molecule refers to the chemical modification of a nucleic acid encoding, or complementary to, the encoded HGPRBMY4 polypeptide. Such modifications include, for example, replacement of hydrogen by an alkyl, acyl, or amino group. A nucleic acid derivative encodes a polypeptide, which retains the essential biological and/or functional characteristics of the natural molecule. A derivative polypeptide is one, which is modified by glycosylation, pegylation, or any similar process that retains the biological and/or functional or immunological activity of the polypeptide from which it is derived.

The term “biologically active,” i.e., functional, refers to a protein or polypeptide or fragment thereof having structural, regulatory, or biochemical functions of a naturally occurring molecule. Likewise, “immunologically active” refers to the capability of the natural, recombinant, or synthetic HGPRBMY4, or any oligopeptide thereof, to induce a specific immune response in appropriate animals or cells, for example, to generate antibodies, and to bind with specific antibodies.

The term “hybridization” refers to any process by which a strand of nucleic acid binds with a complementary strand through base pairing.

The term “hybridization complex” refers to a complex formed between two nucleic acid sequences by virtue of the formation of hydrogen bonds between complementary G and C bases and between complementary A and T bases. The hydrogen bonds can be further stabilized by base stacking interactions. The two complementary nucleic acid sequences hydrogen bond in an anti-parallel configuration. A hybridization complex can be formed in solution (e.g., Cₒt or Rₒt analysis), or between one nucleic acid sequence present in solution and another nucleic acid sequence immobilized on a solid support (e.g., membranes, filters, chips, pins, or glass slides, or any other appropriate substrate to which cells or their nucleic acids have been affixed).

The terms “stringency” or “stringent conditions” refer to the conditions for hybridization as defined by nucleic acid composition, salt and temperature. These conditions are well known in the art and can be altered to identify and/or detect identical or related polynucleotide sequences in a sample. A variety of equivalent conditions comprising either low, moderate, or high stringency depend on factors such as the length and nature of the sequence (DNA, RNA, base composition), reaction milieu (in solution or immobilized on a solid substrate), nature of the target nucleic acid (DNA, RNA, base composition), concentration of salts and the presence or absence of other reaction components (e.g., formamide, dextran sulfate and/or polyethylene glycol) and reaction temperature (within a range of from about 5° C. below the melting temperature of the probe to about 20° C. to 25° C. below the melting temperature). One or more factors can be varied to generate conditions, either low or high stringency, that are different from but equivalent to the aforementioned conditions.

As will be understood by those of skill in the art, the stringency of hybridization can be altered in order to identify or detect identical or related polynucleotide sequences. As will be further appreciated by the skilled practitioner, melting temperature, Tₘ, can be approximated by the formulas as known in the art, depending on a number of parameters, such as the length of the hybrid or probe in number of nucleotides, or hybridization buffer ingredients and conditions (see, for example, T. Maniatis et al., Molecular Cloning: A Laboratory Manual, Cold Spring Harbor Laboratory, Cold Spring Harbor, N.Y., 1982 and J. Sambrook et al., Molecular Cloning: A Laboratory Manual, Cold Spring Harbor Laboratory, Cold Spring Harbor, N.Y., 1989; Current Protocols in Molecular Biology, Eds. F. M. Ausubel et al., Vol. 1, “Preparation and Analysis of DNA,” John Wiley and Sons, Inc., 1994-1995, Suppls. 26, 29, 35 and 42; pp. 2.10.7-2.10.16; G. M. Wahl and S. L. Berger (1987; Methods Enzymol. 152:399-407); and A. R. Kimmel, 1987; Methods of Enzymol. 152:507-511). As a general guide, Tₘ decreases approximately 1° C.-1.5° C. with every 1% decrease in sequence homology. Also, in general, the stability of a hybrid is a function of sodium ion concentration and temperature. Typically, the hybridization reaction is initially performed under conditions of low stringency, followed by washes of varying, but higher stringency. Reference to hybridization stringency, for example, high, moderate, or low stringency, typically relates to such washing conditions.

Thus, by way of non-limiting example, “high stringency” refers to conditions that permit hybridization of those nucleic acid sequences that form stable hybrids in 0.018 M NaCl at about 65° C. (i.e., if a hybrid is not stable in 0.018 M NaCl at about 65° C., it will not be stable under high stringency conditions). High stringency conditions can be provided, for instance, by hybridization in 50% formamide, 5× Denhardt's solution, 5×SSPE (saline sodium phosphate EDTA) (1×SSPE buffer comprises 0.15 M NaCl, 10 mM Na₂HPO₄, 1 mM EDTA), (or 1×SSC buffer containing 150 mM NaCl, 15 mM Na₃ citrate •2 H₂O, pH 7.0), 0.2% SDS at about 42° C., followed by washing in 1×SSPE (or saline sodium citrate, SSC) and 0.1% SDS at a temperature of at least about 42° C., preferably about 55° C., more preferably about 65° C.

“Moderate stringency” refers, by non-limiting example, to conditions that permit hybridization in 50% formamide, 5×Denhardt's solution, 5×SSPE (or SSC), 0.2% SDS at 42° C. (to about 50° C.), followed by washing in 0.2×SSPE (or SSC) and 0.2% SDS at a temperature of at least about 42° C., preferably about 55° C., more preferably about 65° C

“Low stringency” refers, by non-limiting example, to conditions that permit hybridization in 10% formamide, 5×Denhardt's solution, 6×SSPE (or SSC), 0.2% SDS at 42° C., followed by washing in 1×SSPE (or SSC) and 0.2% SDS at a temperature of about 45° C., preferably about 50° C.

For additional stringency conditions, see T. Maniatis et al., Molecular Cloning: A Laboratory Manual, Cold Spring Harbor Laboratory, Cold Spring Harbor, N.Y. (1982). It is to be understood that the low, moderate and high stringency hybridization/washing conditions can be varied using a variety of ingredients, buffers and temperatures well known to and practiced by the skilled artisan.

The terms “complementary” or “complementarity” refer to the natural binding of polynucleotides under permissive salt and temperature conditions by base-pairing. For example, the sequence “A-G-T” binds to the complementary sequence “T-C-A.” Complementarity between two single-stranded molecules can be “partial,” in which only some of the nucleic acids bind, or it can be complete when total complementarity exists between single stranded molecules. The degree of complementarity between nucleic acid strands has significant effects on the efficiency and strength of hybridization between nucleic acid strands. This is of particular importance in amplification reactions, which depend upon binding between nucleic acids strands, as well as in the design and use of PNA molecules.

The term “homology” refers to a degree of complementarity. There can be partial homology or complete homology, wherein complete homology is equivalent to identity. A partially complementary sequence that at least partially inhibits an identical sequence from hybridizing to a target nucleic acid is referred to using the functional term “substantially homologous.” The inhibition of hybridization of the completely complementary sequence to the target sequence can be examined using a hybridization assay (e.g., Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. Nonetheless, conditions of low stringency do not permit non-specific binding; low stringency conditions require that the binding of two sequences to one another be a specific (i.e., selective) interaction. The absence of non-specific binding can be tested by the use of a second target sequence which lacks even a partial degree of complementarity (e.g., less than about 30% identity). In the absence of non-specific binding, the probe will not hybridize to the second non-complementary target sequence.

Those having skill in the art will know how to determine percent identity between or among sequences using, for example, algorithms such as those based on the CLUSTALW computer program (J. D. Thompson et al., 1994, Nucleic Acids Research, 2(22):4673-4680), or FASTDB, (Brutlag et al., 1990, Comp. App. Biosci., 6:237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations.

A “composition comprising a given polynucleotide sequence” refers broadly to any composition containing the given polynucleotide sequence. The composition can comprise a dry formulation or an aqueous solution. Compositions comprising polynucleotide sequence (SEQ ID NO: 1) encoding the HGPRBMY4 polypeptide (SEQ ID NO: 2), or fragments thereof, can be employed as hybridization probes. The probes can be stored in freeze-dried form and can be in association with a stabilizing agent such as a carbohydrate. In hybridizations, the probe can be employed in an aqueous solution containing salts (e.g., NaCl), detergents or surfactants (e.g., SDS) and other components (e.g., Denhardt's solution, dry milk, salmon sperm DNA, and the like).

The term “substantially purified” refers to nucleic acid sequences or amino acid sequences that are removed from their natural environment, isolated or separated, and are at least 60% free, preferably 75% to 85% free, and most preferably 90% or greater free from other components with which they are naturally associated.

The term “sample,” or “biological sample,” is meant to be interpreted in its broadest sense. A biological sample suspected of containing nucleic acids encoding the HGPRBMY4 protein, or fragments thereof, or HGPRBMY4 protein itself, can comprise a body fluid, an extract from cells or tissue, chromosomes isolated from a cell (e.g., a spread of metaphase chromosomes), organelle, or membrane isolated from a cell, a cell, nucleic acid such as genomic DNA (in solution or bound to a solid support such as for Southern analysis), RNA (in solution or bound to a solid support such as for Northern analysis), cDNA (in solution or bound to a solid support), a tissue, a tissue print and the like.

“Transformation” refers to a process by which exogenous DNA enters and changes a recipient cell. It can occur under natural or artificial conditions using various methods well known in the art. Transformation can rely on any known method for the insertion of foreign nucleic acid sequences into a prokaryotic or eukaryotic host cell. The method is selected based on the type of host cell being transformed and can include, but is not limited to, viral infection, electroporation, heat shock, lipofection, and partial bombardment. Such “transformed” cells include stably transformed cells in which the inserted DNA is capable of replication either as an autonomously replicating plasmid or as part of the host chromosome. Transformed cells also include those cells, which transiently express the inserted DNA or RNA for limited periods of time.

The term “nmimetic” refers to a molecule, the structure of which is developed from knowledge of the structure of the HGPRBMY4 protein, or portions thereof, and as such, is able to effect some or all of the actions of the HGPRBMY4 protein.

The term “portion” with regard to a protein (as in “a portion of a given protein”) refers to fragments or segments of that protein. The fragments can range in size from four or five amino acid residues to the entire amino acid sequence minus one amino acid. Thus, a protein “comprising at least a portion of the amino acid sequence of SEQ ID NO: 2” encompasses the full-length human HGPRBMY4 polypeptide, and fragments thereof.

The term “antibody” refers to intact molecules as well as fragments thereof, such as Fab, F(ab′)₂, Fv, which are capable of binding an epitopic or antigenic determinant. Antibodies that bind to HGPRBMY4 polypeptides can be prepared using intact polypeptides or fragments containing small peptides of interest or prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, but are not limited to, bovine serum albumin (BSA), keyhole limpet hemocyanin (KLH), and thyroglobulin. The coupled peptide is then used to immunize the animal (e.g, a mouse, a rat, or a rabbit).

The term “humanized” antibody refers to antibody molecules in which amino acids have been replaced in the non-antigen binding regions in order to more closely resemble a human antibody, while still retaining the original binding capability, for example, as described in U.S. Pat. No. 5,585,089 to C. L. Queen et al.

The term “antigenic determinant” refers to that portion of a molecule that makes contact with a particular antibody (i.e., an epitope). When a protein or fragment of a protein is used to immunize a host animal, numerous regions of the protein can induce the production of antibodies which bind specifically to a given region or three-dimensional structure on the protein. These regions or structures are referred to an antigenic determinants. An antigenic determinant can compete with the intact antigen (i.e., the immunogen used to elicit the immune response) for binding to an antibody.

The terms “specific binding” or “specifically binding” refer to the interaction between a protein or peptide and a binding molecule, such as an agonist, an antagonist, or an antibody. The interaction is dependent upon the presence of a particular structure (i.e., an antigenic determinant or epitope) of the protein that is recognized by the binding molecule. For example, if an antibody is specific for epitope “A,” the presence of a protein containing epitope A (or free, unlabeled A) in a reaction containing labeled “A” and the antibody will reduce the amount of labeled A bound to the antibody.

The term “correlates with expression of a polynucleotide” indicates that the detection of the presence of ribonucleic acid that is similar to SEQ ID NO: 1 by Northern analysis is indicative of the presence of mRNA encoding the HGPRBMY4 polypeptide (SEQ ID NO: 2) in a sample and thereby correlates with expression of the transcript from the polynucleotide encoding the protein.

An “alteration” in the polynucleotide of SEQ ID NO: 1 comprises any alteration in the sequence of the polynucleotides encoding the HGPRBMY4 polypeptide (SEQ ID NO: 2), including deletions, insertions, and point mutations that can be detected using hybridization assays. Included within this definition is the detection of alterations to the genomic DNA sequence which encodes the HGPRBMY4 polypeptide (SEQ ID NO: 2; e.g., by alterations in the pattern of restriction fragment length polymorphisms capable of hybridizing to SEQ ID NO: 2), the inability of a selected fragment of the polypeptide of SEQ ID NO: 2 to hybridize to a sample of genomic DNA (e.g., using allele-specific oligonucleotide probes), and improper or unexpected hybridization, such as hybridization to a locus other than the normal chromosomal locus for the polynucleotide sequence encoding the HGPRBMY4 polypeptide (e.g., using fluorescent in situ hybridization (FISH) to metaphase chromosome spreads).

### Description of the Invention

The present invention provides a novel human member of the G-protein coupled receptor (GPCR) family (HGPRBMY4). Based on sequence homology, the protein HGPRBMY4 is a novel human GPCR. This protein sequence has been predicted to contain seven transmembrane domains, which is a characteristic structural feature of GPCRs. This orphan GPCR is expressed highly in prostate, colon, lung, breast, and moderately in the heart. HGPRBMY4 polypeptides and polynucleotides are useful for diagnosing diseases related to over- and under-expression of HGPRBMY4 proteins by identifying mutations in the HGPRBMY4 gene using HGPRBMY4 probes, or determining HGPRBMY4 protein or mRNA expression levels. HGPRBMY4 polypeptides are also useful for screening compounds, which affect activity of the protein. The invention encompasses the polynucleotide encoding the HGPRBMY4 polypeptide and the use of the HGPRBMY4 polynucleotide or polypeptide, or compositions in thereof, the screening, diagnosis, treatment, or prevention of disorders associated with aberrant or uncontrolled cellular growth and/or function, such as neoplastic diseases (e.g., cancers and tumors), with particular regard to those diseases or disorders related to the prostate, colon, lung, breast, or heart, in addition to vascular tissue disorders.

More specifically, the HGPRBMY4 encoding mRNA is expressed highly in several cell lines. The highest expression is in the lung carcinoma cell line (SHP-77), the colon cell line (LS 174T), and the prostate cell line (LNCAP). Weaker expression is observed in several other colon cell lines (SW403, HT-29, T84, MIP). Significant expression is also found in a single prostate tumor compared to control, as confirmed by immunohistochemistry data showing moderate to strong staining in small subsets of normal prostatic epithelial cells, with most cells staining faintly. In normal tissues, the highest expression is observed in blood vessels and associated tissues. This indicates a potential role in blood flow regulation. Accordingly, diseases that can be treated with HGPRBMY4 include Benign Prostate Hyperplasia, acute heart failure, hypotension, hypertension, angina pectoris, myocardial infarction, psychotic, immune, metabolic, neurological, cardiovascular and other prostate disorders, in addition to, colon, breast, and lung diseases, such as, but not limited to, Crohn's disease, Hirschsprung's disease, colonic carcinoma, inflammatory bowel disease, Chagas' disease, breast cancer, ovarian cancer, endometrium cancer, bronchopulmonary dysplasia, post-inflammatory pseudotumor, and Pancoast's syndrome.

Moreover, the HGPRBMY4 polynucleotides and polypeptides, in addition to modulators thereof, would be useful in the detection, treatment, and/or prevention of a variety of vascular disorders and conditions, which include, but are not limited to miscrovascular disease, vascular leak syndrome, aneurysm, stroke, embolism, thrombosis, coronary artery disease, arteriosclerosis, and/or atherosclerosis. Furthermore, the protein may also be used to determine biological activity, raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

The HGPRBMY4 polypeptide has been shown to be involved in the regulation of mammalian NF-κB and apoptosis pathways (see Example 15). Subjecting cells with an effective amount of a pool of all five HGPRBMY4-specific antisense oligoncleotides resulted in a significant increase in E-selectin expression/activity in HMVEC cells providing convincing evidence that HGPRBMY4 at least regulates the activity and/or expression of E-selectin either directly, or indirectly. Moreover, the results suggest that HGPRBMY4 is involved in the negative regulation of NF-κB/IκBα activity and/or expression, either directly or indirectly. The NFkB/E-selectin assay used is described below and was based upon the analysis of E-selectin activity as a downstream marker for inflammatory/proliferative signal transduction events.

HGPRBMY4 polypeptides are also useful for screening compounds, which affect activity of the protein. Nucleic acids, encoding the HGPRBMY4 protein according to the present invention, were first identified, in Incyte CloneID:998550 from a kidney tumor tissue library, through a computer search for amino acid sequence alignments (see Example 1).

In one of its embodiments, the present invention encompasses a polypeptide comprising the amino acid sequence of SEQ ID NO: 2 as shown in FIG. 1. The HGPRBMY4 polypeptide is 318 amino acids in length and shares amino acid sequence homology the putative G-protein coupled receptor, RA1C. The HGPRBMY4 polypeptide shares 60% identity and 77% similarity with 299 amino acids of the putative G-protein coupled receptor RA1C, wherein “similar” amino acids are those which have the same or similar physical properties and in many cases, the function is conserved with similar residues. For example, amino acids lysine and arginine are similar. Residues such as proline and cysteine do not share any physical property and they are not considered similar. The HGPRBMY4 polypeptide shares 58.3% identity and 66.9% similarity with the Rattus norvegicus putative G-protein coupled receptor RA1C (Ace. No.:O88628); 47% identity and 57.8% similarity with the Mus musculus odorant receptor S18 (Acc. No.:Q9WU89); 43.8% identity and 55.6% similarity with the Mus musculus odorant receptor S46 (Acc. No.:Q9WU93); 47.3% identity and 57.8% similarity with the Mus musculus MOR 3′ BETA3 (Acc. No.:Q9WVD7); 47.5% identity and 62% similarity with the Mus musculus MOR 3′BETA1 (Acc. No.:Q9WVD9); 44.4% identity and 56.9% similarity with the Mus musculus MOR 5′BETA1 (Acc. No.:Q9WVN4); 47% identity and 60.5% similarity with Mus musculus MOR 5′BETA2 (Acc. No.:Q9WVN5); 43.1% identity and 57.2% similarity with human HOR 5′BETA3 (Acc. No.:Q9Y5P1); and 50% identity and 62.2% similarity with the Gallus gallus olfactory receptor-like protein COR3′BETA (Acc. No.:Q9YH55).

Variants of the HGPRBMY4 polypeptide are also encompassed by the present invention. A preferred HGPRBMY4 variant has at least 75% to 80%, more preferably at least 85% to 90%, and even more preferably at least 90% amino acid sequence identity to the amino acid sequence claimed herein, and which retains at least one biological, immunological, or other functional characteristic or activity of HGPRBMY4 polypeptide. Most preferred is a variant having at least 95% amino acid sequence identity to that of SEQ ID NO: 2.

In another embodiment, the present invention encompasses polynucleotides, which encode the HGPRBMY4 polypeptide. Accordingly, any nucleic acid sequence, which encodes the amino acid sequence of the HGPRBMY4 polypeptide, can be used to produce recombinant molecules that express the HGPRBMY4 protein. In a particular embodiment, the present invention encompasses the HGPRBMY4 polynucleotide comprising the nucleic acid sequence of SEQ ID NO: 1 and as shown in FIG. 1. More particularly, the present invention provides the HGPRBMY4 clone, deposited at the American Type Culture Collection (ATCC), 10801 University Boulevard, Manassas, Va. 20110-2209 on Nov. 15, 2000 and under ATCC Accession No. PTA-2682 according to the terms of the Budapest Treaty.

As will be appreciated by the skilled practitioner in the art, the degeneracy of the genetic code results in the production of a multitude of nucleotide sequences encoding the HGPRBMY4 polypeptide. Some of the sequences bear minimal homology to the nucleotide sequences of any known and naturally occurring gene. Accordingly, the present invention contemplates each and every possible variation of nucleotide sequence that could be made by selecting combinations based on possible codon choices. These combinations are made in accordance with the standard triplet genetic code as applied to the nucleotide sequence of naturally occurring HGPRBMY4, and all such variations are to be considered as being specifically disclosed.

Although nucleotide sequences which encode the HGPRBMY4 polypeptide and its variants are preferably capable of hybridizing to the nucleotide sequence of the naturally occurring HGPRBMY4 polypeptide under appropriately selected conditions of stringency, it can be advantageous to produce nucleotide sequences encoding the HGPRBMY4 polypeptide, or its derivatives, which possess a substantially different codon usage. Codons can be selected to increase the rate at which expression of the peptide or polypeptide occurs in a particular prokaryotic or eukaryotic host in accordance with the frequency with which particular codons are utilized by the host. Other reasons for substantially altering the nucleotide sequence encoding the HGPRBMY4 polypeptide, and its derivatives, without altering the encoded amino acid sequences include the production of RNA transcripts having more desirable properties, such as a greater half-life, than transcripts produced from the naturally occurring sequence.

The present invention also encompasses production of DNA sequences, or portions thereof, which encode the HGPRBMY4 polypeptide, and its derivatives, entirely by synthetic chemistry. After production, the synthetic sequence can be inserted into any of the many available expression vectors and cell systems using reagents that are well known and practiced by those in the art. Moreover, synthetic chemistry can be used to introduce mutations into a sequence encoding the HGPRBMY4 polypeptide, or any fragment thereof.

Also encompassed by the present invention are polynucleotide sequences that are capable of hybridizing to the claimed nucleotide sequence of HGPRBMY4, such as that shown in SEQ ID NO: 1, under various conditions of stringency. Hybridization conditions are typically based on the melting temperature (Tₘ) of the nucleic acid binding complex or probe (see, G. M. Wahl and S. L. Berger, 1987; Methods Enzymol., 152:399-407 and A. R. Kimmel, 1987; Methods of Enzymol., 152:507-511), and can be used at a defined stringency. For example, included in the present invention are sequences capable of hybridizing under moderately stringent conditions to the HGPRBMY4 polypeptide sequence of SEQ ID NO: 2 and other sequences which are degenerate to those which encode HGPRBMY4 polypeptide (e.g., as a non-limiting example: prewashing solution of 2×SSC, 0.5% SDS, 1.0 mM EDTA, pH 8.0, and hybridization conditions of 50° C., 5×SSC, overnight.

The nucleic acid sequence encoding the HGPRBMY4 protein can be extended utilizing a partial nucleotide sequence and employing various methods known in the art to detect upstream sequences such as promoters and regulatory elements. For example, one method, which can be employed, is restriction-site PCR, which utilizes universal primers to retrieve unknown sequence adjacent to a known locus (G. Sarkar, 1993, PCR Methods Applic., 2:318-322). In particular, genomic DNA is first amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR can also be used to amplify or extend sequences using divergent primers based on a known region or sequence (T. Triglia et al., 1988, Nucleic Acids Res., 16:8186). The primers can be designed using OLIGO 4.06 Primer Analysis software (National Biosciences Inc.; Plymouth, Minn.), or another appropriate program, to be 22-30 nucleotides in length, to have a GC content of 50% or more, and to anneal to the target sequence at temperatures about 68° C.-72° C. The method uses several restriction enzymes to generate a suitable fragment in the known region of a gene. The fragment is then circularized by intramolecular ligation and used as a PCR template.

Another method which can be used is capture PCR which involves PCR amplification of DNA fragments adjacent to a known sequence in human and yeast artificial chromosome (YAC) DNA (M. Lagerstrom et al., 1991, PCR Methods Applic., 1:111-119). In this method, multiple restriction enzyme digestions and ligations can also be used to place an engineered double-stranded sequence into an unknown portion of the DNA molecule before performing PCR. J. D. Parker et al. (1991; Nucleic Acids Res., 19:3055-3060) provide another method which can be used to retrieve unknown sequences. In addition, PCR, nested primers, and PROMOTERFINDER libraries can be used to walk genomic DNA (Clontech; Palo Alto, Calif.). This process avoids the need to screen libraries and is useful in finding intron/exon junctions.

When screening for full-length cDNAs, it is preferable to use libraries that have been size-selected to include larger cDNAs. Also, randomly primed libraries are preferable, since they will contain more sequences, which contain the 5′ regions of genes. The use of a randomly primed library can be especially preferable for situations in which an oligo d(T) library does not yield a full-length cDNA. Genomic libraries can be useful for extension of sequence into the 5′ and 3′ non-transcribed regulatory regions.

The embodiments of the present invention can be practiced using methods for DNA sequencing which are well known and generally available in the art. The methods can employ such enzymes as the Klenow fragment of DNA polymerase I, SEQUENASE (US Biochemical Corp. Cleveland, Ohio), Taq polymerase (PE Biosystems), thermostable T7 polymerase (Amersham Pharmacia Biotech, Piscataway, N.J.), or combinations of recombinant polymerases and proofreading exonucleases such as the ELONGASE Amplification System marketed by Life Technologies (Gaithersburg, Md.). Preferably, the process is automated with machines such as the Hamilton Micro Lab 2200 (Hamilton, Reno, Nev.), Peltier Thermal Cycler (PTC200; MJ Research; Watertown, Mass.) and the ABI Catalyst and 373 and 377 DNA sequencers (PE Biosystems).

Commercially available capillary electrophoresis systems can be used to analyze the size or confirm the nucleotide sequence of sequencing or PCR products. In particular, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g., GENOTYPER and SEQUENCE NAVIGATOR, PE Biosystems) and the entire process—from loading of samples to computer analysis and electronic data display—can be computer controlled. Capillary electrophoresis is especially preferable for the sequencing of small pieces of DNA, which can be present in limited amounts in a particular sample.

In another embodiment of the present invention, polynucleotide sequences or fragments thereof which encode the HGPRBMY4 polypeptide, or peptides thereof, can be used in recombinant DNA molecules to direct the expression of the HGPRBMY4 polypeptide product, or fragments or functional equivalents thereof, in appropriate host cells. Because of the inherent degeneracy of the genetic code, other DNA sequences, which encode substantially the same or a functionally equivalent amino acid sequence, can be produced and these sequences can be used to clone and express the HGPRBMY4 protein.

As will be appreciated by those having skill in the art, it can be advantageous to produce HGPRBMY4 polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

The nucleotide sequence of the present invention can be engineered using methods generally known in the art in order to alter HGPRBMY4 polypeptide-encoding sequences for a variety of reasons, including, but not limited to, alterations which modify the cloning, processing, and/or expression of the gene product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, or introduce mutations, and the like.

In preferred embodiments, the present invention encompasses a polynucleotide lacking the initiation start codon, in addition to, the resulting encoded polypeptide of HGPRBMY4. Specifically, the present invention encompasses the polynucleotide of nucleotides 4 through 954 of SEQ ID NO: 1, and the polypeptide of amino acids 2 through 318 of SEQ ID NO: 2. Also encompassed are recombinant vectors comprising said encoding sequence, and host cells comprising said vector.

In another embodiment of the present invention, natural, modified, or recombinant nucleic acid sequences encoding the HGPRBMY4 polypeptide can be ligated to a heterologous sequence to encode a fusion protein. For example, for screening peptide libraries for inhibitors of HGPRBMY4 activity, it can be useful to encode a chimeric HGPRBMY4 protein that can be recognized by a commercially available antibody. A fusion protein can also be engineered to contain a cleavage site located between the HGPRBMY4 protein-encoding sequence and the heterologous protein sequence, so that HGPRBMY4 protein can be cleaved and purified away from the heterologous moiety.

In another embodiment, sequences encoding HGPRBMY4 polypeptide can be synthesized in whole, or in part, using chemical methods well known in the art (see, for example, M. H. Caruthers et al., 1980, Nucl. Acids Res. Symp. Ser., 215-223 and T. Horn et al., 1980, Nucl. Acids Res. Symp. Ser., 225-232). Alternatively, the protein itself can be produced using chemical methods to synthesize the amino acid sequence of HGPRBMY4 polypeptide, or a fragment or portion thereof. For example, peptide synthesis can be performed using various solid-phase techniques (J. Y. Roberge et al., 1995, Science, 269:202-204) and automated synthesis can be achieved, for example, using the ABI 431A Peptide Synthesizer (PE Biosystems).

The newly synthesized peptide can be substantially purified by preparative high performance liquid chromatography (e.g., T. Creighton, 1983, Proteins, Structures and Molecular Principles, W. H. Freeman and Co., New York, N.Y.), by reversed-phase high performance liquid chromatography, or other purification methods as are known in the art. The composition of the synthetic peptides can be confirmed by amino acid analysis or sequencing (e.g., the Edman degradation procedure; Creighton, supra). In addition, the amino acid sequence of HGPRBMY4 polypeptide or any portion thereof, can be altered during direct synthesis and/or combined using chemical methods with sequences from other proteins, or any part thereof, to produce a variant polypeptide.

To express a biologically active HGPRBMY4 polypeptide or peptide, the nucleotide sequences encoding HGPRBMY4 polypeptide, or functional equivalents, can be inserted into an appropriate expression vector, i.e., a vector which contains the necessary elements for the transcription and translation of the inserted coding sequence.

Methods, which are well known to those skilled in the art, can be used to construct expression vectors containing sequences encoding HGPRBMY4 polypeptide and appropriate transcriptional and translational control elements. These methods include in vitro recombinant DNA techniques, synthetic techniques, and in vivo genetic recombination. Such techniques are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

A variety of expression vector/host systems can be utilized to contain and express sequences encoding HGPRBMY4 polypeptide. Such expression vector/host systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., bacculovirus); plant cell systems transformed with virus expression vectors (e.g., cauliflower mosaic virus (CaMV) and tobacco mosaic virus (TMV)), or with bacterial expression vectors (e.g., Ti or pBR322 plasmids); or animal cell systems. The host cell employed is not limiting to the present invention.

“Control elements” or “regulatory sequences” are those non-translated regions of the vector, for example, enhancers, promoters, 5′ and 3′ untranslated regions, which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, La Jolla, Calif.) or PSPORT1 plasmid (Life Technologies), and the like, can be used. The baculovirus polyhedrin promoter can be used in insect cells. Promoters or enhancers derived from the genomes of plant cells (e.g., heat shock, RUBISCO; and storage protein genes), or from plant viruses (e.g., viral promoters or leader sequences), can be cloned into the vector. In mammalian cell systems, promoters from mammalian genes or from mammalian viruses are preferred. If it is necessary to generate a cell line that contains multiple copies of the sequence encoding HGPRBMY4, vectors based on SV40 or EBV can be used with an appropriate selectable marker.

In bacterial systems, a number of expression vectors can be selected, depending upon the use intended for the expressed HGPRBMY4 product. For example, when large quantities of expressed protein are needed for the induction of antibodies, vectors, which direct high level expression of fusion proteins that are readily purified, can be used. Such vectors include, but are not limited to, the multifunctional E. coli cloning and expression vectors such as BLUESCRIPT (Stratagene), in which the sequence encoding HGPRBMY4 polypeptide can be ligated into the vector in-frame with sequences for the amino-terminal Met and the subsequent 7 residues of β-galactosidase, so that a hybrid protein is produced; pIN vectors (see, G. Van Heeke and S. M. Schuster, 1989, J. Biol. Chem., 264:5503-5509); and the like. pGEX vectors (Promega, Madison, Wis.) can also be used to express foreign polypeptides, as fusion proteins with glutathione S-transferase (GST). In general, such fusion proteins are soluble and can be easily purified from lysed cells by adsorption to glutathione-agarose beads followed by elution in the presence of free glutathione. Proteins made in such systems can be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH can be used. (For reviews, see F. M. Ausubel et al., supra, and Grant et al., 1987, Methods Enzymol., 153:516-544).

Should plant expression vectors be desired and used, the expression of sequences encoding HGPRBMY4 polypeptide can be driven by any of a number of promoters. For example, viral promoters such as the 35S and 19S promoters of CaMV can be used alone or in combination with the omega leader sequence from TMV (N. Takamatsu, 1987, EMBO J., 6:307-311). Alternatively, plant promoters such as the small subunit of RUBISCO, or heat shock promoters, can be used (G. Coruzzi et al., 1984, EMBO J., 3:1671-1680; R. Broglie et al., 1984, Science, 224:838-843; and J. Winter et al., 1991, Results Probl. Cell Differ. 17:85-105). These constructs can be introduced into plant cells by direct DNA transformation or pathogen-mediated transfection. Such techniques are described in a number of generally available reviews (see, for example, S. Hobbs or L. E. Murry, In: McGraw Hill Yearbook of Scienc and Technology (1992) McGraw Hill, New York, N.Y.; pp. 191-196).

An insect system can also be used to express HGPRBMY4 polypeptide. For example, in one such system, Autographa califomica nuclear polyhedrosis virus (AcNPV) is used as a vector to express foreign genes in Spodoptera frugiperda cells or in Trichoplusia larvae. The sequences encoding HGPRBMY4 polypeptide can be cloned into a non-essential region of the virus such as the polyhedrin gene and placed under control of the polyhedrin promoter. Successful insertion of HGPRBMY4 polypeptide will render the polyhedrin gene inactive and produce recombinant virus lacking coat protein. The recombinant viruses can then be used to infect, for example, S. frugiperda cells or Trichoplusia larvae in which the HGPRBMY4 polypeptide product can be expressed (E. K. Engelhard et al., 1994, Proc. Nat. Acad. Sci., 91:3224-3227).

In mammalian host cells, a number of viral-based expression systems can be utilized. In cases where an adenovirus is used as an expression vector, sequences encoding HGPRBMY4 polypeptide can be ligated into an adenovirus transcription/translation complex containing the late promoter and tripartite leader sequence. Insertion in a non-essential E1 or E3 region of the viral genome can be used to obtain a viable virus which is capable of expressing HGPRBMY4 polypeptide in infected host cells (J. Logan and T. Shenk, 1984, Proc. Natl. Acad. Sci., 81:3655-3659). In addition, transcription enhancers, such as the Rous sarcoma virus (RSV) enhancer, can be used to increase expression in mammalian host cells.

Specific initiation signals can also be used to achieve more efficient translation of sequences encoding HGPRBMY4 polypeptide. Such signals include the ATG initiation codon and adjacent sequences. In cases where sequences encoding HGPRBMY4 polypeptide, its initiation codon, and upstream sequences are inserted into the appropriate expression vector, no additional transcriptional or translational control signals can be needed. However, in cases where only coding sequence, or a fragment thereof, is inserted, exogenous translational control signals, including the ATG initiation codon, should be provided. Furthermore, the initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system that is used, such as those described in the literature (D. Scharf et al., 1994, Results Probl. Cell Differ., 20:125-162).

Moreover, a host cell strain can be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein can also be used to facilitate correct insertion, folding and/or function. Different host cells having specific cellular machinery and characteristic mechanisms for such post-translational activities (e.g., CHO, HeLa, MDCK, HEK293, and W138) are available from the American Type Culture Collection (ATCC), American Type Culture Collection (ATCC), 10801 University Boulevard, Manassas, Va. 20110-2209, and can be chosen to ensure the correct modification and processing of the foreign protein.

For long-term, high-yield production of recombinant proteins, stable expression is preferred. For example, cell lines which stably express HGPRBMY4 protein can be transformed using expression vectors which can contain viral origins of replication and/or endogenous expression elements and a selectable marker gene on the same, or on a separate, vector. Following the introduction of the vector, cells can be allowed to grow for 1-2 days in an enriched cell culture medium before they are switched to selective medium. The purpose of the selectable marker is to confer resistance to selection, and its presence allows the growth and recovery of cells, which successfully express the introduced sequences. Resistant clones of stably transformed cells can be proliferated using tissue culture techniques appropriate to the cell type.

Any number of selection systems can be used to recover transformed cell lines. These include, but are not limited to, the Herpes Simplex Virus thymidine kinase (HSV TK), (M. Wigler et al., 1977, Cell, 11:223-32) and adenine phosphoribosyltransferase (I. Lowy et al., 1980, Cell, 22:817-23) genes which can be employed in tk- or aprt-cells, respectively. Also, anti-metabolite, antibiotic or herbicide resistance can be used as the basis for selection; for example, dhfr, which confers resistance to methotrexate (M. Wigler et al., 1980, Proc. Natl. Acad. Sci., 77:3567-70); npt, which confers resistance to the aminoglycosides neomycin and G-418 (F. Colbere-Garapin et al., 1981, J. Mol. Biol., 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (S. C. Hartman and R. C. Mulligan, 1988, Proc. Natl. Acad. Sci., 85:8047-51). Recently, the use of visible markers has gained popularity with such markers as the anthocyanins, β-glucuronidase and its substrate GUS, and luciferase and its substrate luciferin, which are widely used not only to identify transformants, but also to quantify the amount of transient or stable protein expression that is attributable to a specific vector system (C. A. Rhodes et al., 1995, Methods Mol. Biol., 55:121-131).

Although the presence or absence of marker gene expression suggests that the gene of interest is also present, the presence and expression of the desired gene of interest can need to be confirmed. For example, if the nucleic acid sequence encoding the HGPRBMY4 polypeptide is inserted within a marker gene sequence, recombinant cells containing sequences encoding the HGPRBMY4 polypeptide can be identified by the absence of marker gene function. Alternatively, a marker gene can be placed in tandem with a sequence encoding the HGPRBMY4 polypeptide under the control of a single promoter. Expression of the marker gene in response to induction or selection usually indicates co-expression of the tandem gene.

Alternatively, host cells, which contain the nucleic acid, sequence encoding the HGPRBMY4 polypeptide and which express HGPRBMY4 polypeptide product can be identified by a variety of procedures known to those having skill in the art. These procedures include, but are not limited to, DNA-DNA or DNA-RNA hybridizations and protein bioassay or immunoassay techniques, including membrane, solution, or chip based technologies, for the detection and/or quantification of nucleic acid or protein.

The presence of polynucleotide sequences encoding the HGPRBMY4 polypeptide can be detected by DNA-DNA or DNA-RNA hybridization, or by amplification using probes or portions or fragments of polynucleotides encoding the HGPRBMY4 polypeptide. Nucleic acid amplification based assays involve the use of oligonucleotides or oligomers, based on the sequences encoding the HGPRBMY4 polypeptide, to detect transformants containing DNA or RNA encoding the HGPRBMY4 polypeptide.

A wide variety of labels and conjugation techniques are known and employed by those skilled in the art and can be used in various nucleic acid and amino acid assays. Means for producing labeled hybridization or PCR probes for detecting sequences related to polynucleotides encoding HGPRBMY4 polypeptide include oligo-labeling, nick translation, end-labeling, or PCR amplification using a labeled nucleotide. Alternatively, the sequences encoding HGPRBMY4 polypeptide, or any portions or fragments thereof, can be cloned into a vector for the production of an mRNA probe. Such vectors are known in the art, are commercially available, and can be used to synthesize RNA probes in vitro by addition of an appropriate RNA polymerase, such as T7, T3, or SP(6) and labeled nucleotides. These procedures can be conducted using a variety of commercially available kits (e.g., Amersham Pharmacia Biotech, Promega, and U.S. Biochemical Corp.). Suitable reporter molecules or labels which can be used include radionuclides, enzymes, fluorescent, chemiluminescent, or chromogenic agents, as well as substrates, cofactors, inhibitors, magnetic particles, and the like.

Host cells transformed with nucleotide sequences encoding HGPRBMY4 protein, or fragments thereof, can be cultured under conditions suitable for the expression and recovery of the protein from cell culture. The protein produced by a recombinant cell can be secreted or contained intracellularly depending on the sequence and/or the vector used. As will be understood by those having skill in the art, expression vectors containing polynucleotides which encode the HGPRBMY4 protein can be designed to contain signal sequences which direct secretion of the HGPRBMY4 protein through a prokaryotic or eukaryotic cell membrane. Other constructions can be used to join nucleic acid sequences encoding the HGPRBMY4 protein to nucleotide sequence encoding a polypeptide domain, which will facilitate purification of soluble proteins. Such purification facilitating domains include, but are not limited to, metal chelating peptides such as histidine-tryptophan modules that allow purification on immobilized metals; protein A domains that allow purification on immobilized immunoglobulin; and the domain utilized in the FLAGS extension/affinity purification system (Immunex Corp.; Seattle, Wash.). The inclusion of cleavable linker sequences such as those specific for Factor XA or enterokinase (Invitrogen; San Diego, Calif.) between the purification domain and HGPRBMY4 protein can be used to facilitate purification. One such expression vector provides for expression of a fusion protein containing HGPRBMY4 and a nucleic acid encoding 6 histidine residues preceding a thioredoxin or an enterokinase cleavage site. The histidine residues facilitate purification on IMAC (immobilized metal ion affinity chromatography) as described by J. Porath et al., 1992, Prot. Exp. Purif., 3:263-281, while the enterokinase cleavage site provides a means for purifying from the fusion protein. For a discussion of suitable vectors for fusion protein production, see D. J. Kroll et al., 1993; DNA Cell Biol., 12:441-453.

In addition to recombinant production, fragments of HGPRBMY4 polypeptide can be produced by direct peptide synthesis using solid-phase techniques (J. Merrifield, 1963, J. Am. Chem. Soc., 85:2149-2154). Protein synthesis can be performed using manual techniques or by automation. Automated synthesis can be achieved, for example, using ABI 431A Peptide Synthesizer (PE Biosystems). Various fragments of HGPRBMY4 polypeptide can be chemically synthesized separately and then combined using chemical methods to produce the full length molecule.

Human artificial chromosomes (HACs) can be used to deliver larger fragments of DNA than can be contained and expressed in a plasmid vector. HACs are linear microchromosomes which can contain DNA sequences of 10K to 10M in size, and contain all of the elements that are required for stable mitotic chromosome segregation and maintenance (see, J. J. Harrington et al., 1997, Nature Genet., 15:345-355). HACs of 6 to 10M are constructed and delivered via conventional delivery methods (e.g., liposomes, polycationic amino polymers, or vesicles) for therapeutic purposes.

### Diagnostic Assays

A variety of protocols for detecting and measuring the expression of the HGPRBMY4 polypeptide using either polyclonal or monoclonal antibodies specific for the protein are known and practiced in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive with two non-interfering epitopes on the HGPRBMY4 polypeptide is preferred, but a competitive binding assay can also be employed. These and other assays are described in the art as represented by the publications of R. Hampton et al., 1990; Serological Methods, a Laboratory Manual, APS Press, St. Paul, Minn. and D. E. Maddox et al., 1983; J. Exp. Med., 158:1211-1216.

This invention also relates to the use of HGPRBMY4 polynucleotides as diagnostic reagents. Detection of a mutated form of the HGPRBMY4 gene associated with a dysfunction will provide a diagnostic tool that can add to or define a diagnosis of a disease or susceptibility to a disease which results from under-expression, over-expression, or altered expression of HGPRBMY4. Individuals carrying mutations in the HGPRBMY4 gene can be detected at the DNA level by a variety of techniques.

Nucleic acids for diagnosis can be obtained from a subject's cells, such as from, but not limited to blood, urine, saliva, tissue biopsy or autopsy material. The genomic DNA can be used directly for detection or can be amplified enzymatically by using PCR or other amplification techniques prior to analysis. RNA or cDNA can also be used in similar fashion. Deletions and insertions can be detected by a change in size of the amplified product in comparison to the normal genotype. Hybridizing amplified DNA to labeled HGPRBMY4 polynucleotide sequences can identify point mutations. Perfectly matched sequences can be distinguished from mismatched duplexes by RNase digestion or by differences in melting temperatures. DNA sequence differences can also be detected by alterations in electrophoretic mobility of DNA fragments in gels, with or without denaturing agents, or by direct DNA sequencing. (See, e.g., Myers et al., Science (1985) 230:1242). Sequence changes at specific locations can also be revealed by nuclease protection assays, such as RNase and S1 protection or the chemical cleavage method (see Cotton et al., Proc. Natl. Acad. Sci., USA (1985) 85:43297-4401). In another embodiment, an array of oligonucleotides probes comprising the HGPRBMY4 nucleotide sequence or fragments thereof can be constructed to conduct efficient screening of, for example, genetic mutations. Array technology methods are well known and have general applicability and can be used to address a variety of questions in molecular genetics including gene expression, genetic linkage, and genetic variability (see e.g., M. Chee et al., Science, 274:610-613, 1996).

The diagnostic assays offer a process for diagnosing or determining a susceptibility to infections such as bacterial, fungal, protozoan and viral infections, particularly infections caused by HIV-1 or HIV-2 through detection of a mutation in the HGPRBMY4 gene by the methods described. The invention also provides diagnostic assays for determining or monitoring susceptibility to the following conditions, diseases, or disorders: cancers; anorexia; bulimia asthma; Parkinson's disease; acute heart failure; hypotension; hypertension; urinary retention; osteoporosis; angina pectoris; myocardial infarction; ulcers; asthma; allergies; benign prostatic hypertrophy; prostate intraepithelial neoplasm; and psychotic and neurological disorders, including anxiety, schizophrenia, manic depression, delirium, dementia, severe mental retardation and dyskinesias, such as Huntington's disease or Gilles dela Tourett's syndrome.

In addition, infections such as bacterial, protozoan and viral infections, particularly infections caused by HIV-1 or HIV-2; as well as, conditions or disorders such as pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; prostate intraepithelial neoplasms; and psychotic and neurological disorders, including anxiety, schizophrenia, manic depression, delirium, dementia, severe mental retardation and dyskinesias, such as Huntington's disease or Gilles dela Tourett's syndrome, can be diagnosed by methods comprising determining from a sample derived from a subject having an abnormally decreased or increased level of the HGPRBMY4 polypeptide (SEQ ID NO: 2) or HGPRBMY4 mRNA. Decreased or increased expression can be measured at the RNA level using any of the methods well known in the art for the quantification of polynucleotides, such as, for example, PCR, RT-PCR, RNase protection, Northern blotting and other hybridization methods. Assay techniques that can be used to determine levels of a protein, such as an HGPRBMY4, in a sample derived from a host are well known to those of skill in the art. Such assay methods include radioimmunoassays, competitive-binding assays, Western Blot analysis, and ELISA assays.

In another of its aspects, the present invention relates to a diagnostic kit for a disease or susceptibility to a disease, particularly infections such as bacterial, fungal, protozoan and viral infections, particularly infections caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy, prostate intraepithelial neoplasms, and psychotic and neurological disorders, including anxiety, schizophrenia, manic depression, delirium, dementia, severe medal retardation and dyskinesias, such as Huntington's disease or Gilles dela Tourett's syndrome, which comprises:

(a) a HGPRBMY4 polynucleotide, preferably the nucleotide sequence of SEQ ID NO: 1, or a fragment thereof; or

(b) a nucleotide sequence complementary to that of (a); or

(c) a HGPRBMY4 polypeptide, preferably the polypeptide of SEQ ID NO: 2, or a fragment thereof; or

(d) an antibody to a HGPRBMY4 polypeptide, preferably to the polypeptide of SEQ ID NO: 2, or combinations thereof. It will be appreciated that in any such kit, (a), (b), (c) or (d) can comprise a substantial component.

The GPCR polynucleotides which can be used in the diagnostic assays according to the present invention include oligonucleotide sequences, complementary RNA and DNA molecules, and PNAs. The polynucleotides can be used to detect and quantify the HGPRBMY4-encoding nucleic acid expression in biopsied tissues in which expression (or under- or over-expression) of the HGPRBMY4 polynucleotide can be correlated with disease. The diagnostic assays can be used to distinguish between the absence, presence, and excess expression of HGPRBMY4, and to monitor regulation of HGPRBMY4 polynucleotide levels during therapeutic treatment or intervention.

In a related aspect, hybridization with PCR probes which are capable of detecting polynucleotide sequences, including genomic sequences, encoding the HGPRBMY4 polypeptide, or closely related molecules, can be used to identify nucleic acid sequences which encode the HGPRBMY4 polypeptide. The specificity of the probe, whether it is made from a highly specific region, for example, about 8 to 10 contiguous nucleotides in the 5′ regulatory region, or a less specific region, for example, especially in the 3′ coding region, and the stringency of the hybridization or amplification (maximal, high, intermediate, or low) will determine whether the probe identifies only naturally occurring sequences encoding HGPRBMY4 polypeptide, alleles thereof, or related sequences.

Probes can also be used for the detection of related sequences, and should preferably contain at least 50% of the nucleotides encoding the HGPRBMY4 polypeptide. The hybridization probes of this invention can be DNA or RNA and can be derived from the nucleotide sequence of SEQ ID NO: 1, or from genomic sequence including promoter, enhancer elements, and introns of the naturally occurring HGPRBMY4 protein.

Methods for producing specific hybridization probes for DNA encoding the HGPRBMY4 polypeptide include the cloning of a nucleic acid sequence that encodes the HGPRBMY4 polypeptide, or HGPRBMY4 derivatives, into vectors for the production of mRNA probes. Such vectors are known in the art, commercially available, and can be used to synthesize RNA probes in vitro by means of the addition of the appropriate RNA polymerases and the appropriate labeled nucleotides. Hybridization probes can be labeled by a variety of detector or reporter groups, for example, radionuclides such as ³²P or ³⁵S, or enzymatic labels, such as alkaline phosphatase coupled to the probe via avidin/biotin coupling systems, and the like.

The polynucleotide sequence encoding the HGPRBMY4 polypeptide, or fragments thereof, can be used for the diagnosis of disorders associated with expression of HGPRBMY4. Examples of such disorders or conditions are described above for “Therapeutics.” The polynucleotide sequence encoding the HGPRBMY4 polypeptide can be used in Southern or Northern analysis, dot blot, or other membrane-based technologies; in PCR technologies; or in dip stick, pin, ELISA or chip assays utilizing fluids or tissues from patient biopsies to detect the status of, for example, levels or overexpression of HGPRBMY4, or to detect altered HGPRBMY4 expression. Such qualitative or quantitative methods are well known in the art.

In a particular aspect, the nucleotide sequence encoding the HGPRBMY4 polypeptide can be useful in assays that detect activation or induction of various neoplasms or cancers, particularly those mentioned supra. The nucleotide sequence encoding the HGPRBMY4 polypeptide can be labeled by standard methods, and added to a fluid or tissue sample from a patient, under conditions suitable for the formation of hybridization complexes. After a suitable incubation period, the sample is washed and the signal is quantified and compared with a standard value. If the amount of signal in the biopsied or extracted sample is significantly altered from that of a comparable control sample, the nucleotide sequence has hybridized with nucleotide sequence present in the sample, and the presence of altered levels of nucleotide sequence encoding the HGPRBMY4 polypeptide in the sample indicates the presence of the associated disease. Such assays can also be used to evaluate the efficacy of a particular therapeutic treatment regimen in animal studies, in clinical trials, or in monitoring the treatment of an individual patient.

To provide a basis for the diagnosis of disease associated with expression of HGPRBMY4, a normal or standard profile for expression is established. This can be accomplished by combining body fluids or cell extracts taken from normal subjects, either animal or human, with a sequence, or a fragment thereof, which encodes the HGPRBMY4 polypeptide, under conditions suitable for hybridization or amplification. Standard hybridization can be quantified by comparing the values obtained from normal subjects with those from an experiment where a known amount of a substantially purified polynucleotide is used. Standard values obtained from normal samples can be compared with values obtained from samples from patients who are symptomatic for disease. Deviation between standard and subject (patient) values is used to establish the presence of disease.

Once disease is established and a treatment protocol is initiated, hybridization assays can be repeated on a regular basis to evaluate whether the level of expression in the patient begins to approximate that which is observed in a normal individual. The results obtained from successive assays can be used to show the efficacy of treatment over a period ranging from several days to months.

With respect to cancer, the presence of an abnormal amount of transcript in biopsied tissue from an individual can indicate a predisposition for the development of the disease, or can provide a means for detecting the disease prior to the appearance of actual clinical symptoms. A more definitive diagnosis of this type can allow health professionals to employ preventative measures or aggressive treatment earlier, thereby preventing the development or further progression of the cancer.

Additional diagnostic uses for oligonucleotides designed from the nucleic acid sequence encoding the HGPRBMY4 polypeptide can involve the use of PCR. Such oligomers can be chemically synthesized, generated enzymatically, or produced from a recombinant source. Oligomers will preferably comprise two nucleotide sequences, one with sense orientation (5′→3′) and another with antisense (3′→5′), employed under optimized conditions for identification of a specific gene or condition. The same two oligomers, nested sets of oligomers, or even a degenerate pool of oligomers can be employed under less stringent conditions for detection and/or quantification of closely related DNA or RNA sequences.

Methods suitable for quantifying the expression of HGPRBMY4 include radiolabeling or biotinylating nucleotides, co-amplification of a control nucleic acid, and standard curves onto which the experimental results are interpolated (P. C. Melby et al., 1993, J. Immunol. Methods, 159:235-244; and C. Duplaa et al., 1993, Anal. Biochem., 229-236). The speed of quantifying multiple samples can be accelerated by running the assay in an ELISA format where the oligomer of interest is presented in various dilutions and a spectrophotometric or colorimetric response gives rapid quantification.

### Therapeutic Assays

The HGPRBMY4 polypeptide shares homology with a putative G-protein coupled receptor (RA1C). As determined by expression in various tissues, HGPRBMY4 can play a role in prostate-, colon-, lung-, breast-, or cardiovascular-related disorders, and in cell signaling or cell cycle regulation. The HGPRBMY4 protein may be further involved in neoplastic and neurological-related disorders, where it may also be associated with cell cycle and cell signaling activities, as described further below.

In one embodiment of the present invention, the HGPRBMY4 protein can play a role in neoplastic disorders. An antagonist of the HGPRBMY4 polypeptide can be administered to an individual to prevent or treat a neoplastic disorder. Such disorders can include, but are not limited to, adenocarcinoma, leukemia, lymphoma, melanoma, myeloma, sarcoma, and teratocarcinoma, and particularly, cancers of the adrenal gland, bladder, bone, bone marrow, brain, breast, cervix, colon, endometrium, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In a related aspect, an antibody which specifically binds to HGPRBMY4 can be used directly as an antagonist or indirectly as a targeting or delivery mechanism for bringing a pharmaceutical agent to cells or tissue which express the HGPRBMY4 polypeptide.

In another embodiment of the present invention, an antagonist or inhibitory agent of the HGPRBMY4 polypeptide can be administered to a subject to prevent or treat a neurological disorder. Such disorders can include, but are not limited to, akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, Parkinson's disease, paranoid psychoses, schizophrenia, and Tourette's disorder.

In another embodiment of the present invention, an antagonist or inhibitory agent of the HGPRBMY4 polypeptide can be administered to an individual to prevent or treat an immune disorder. Such disorders can include, but are not limited to, AIDS, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjögren's syndrome, and autoimmune thyroiditis; complications of cancer, hemodialysis, extracorporeal circulation; viral, bacterial, fungal, parasitic, protozoal, and helminthic infections and trauma.

In a preferred embodiment of the present invention, an antagonist or inhibitory agent of the HGPRBMY4 polypeptide can be administered to an individual to prevent or treat a prostate-, colon-, lung-, breast-, and cardiovascular-related disorder, particularly since HGPRBMY4 is highly expressed in prostate, colon, breast, and lung, while moderately expressed in the heart. Such conditions or disorders can include, but are not limited to, prostatitis, benign prostatic hyperplasia, prostate intraepithelial neoplasms, urogenital cancers, Crohn's disease, Hirschsprung's disease, inflammatory bowel disease, Chagas' disease, bronchopulmonary disease, post-inflammatory pseudotumor, Pancoast's syndrome, and cardiovascular diseases.

In preferred embodiments, the HGPRBMY4 polynucleotides and polypeptides, including agonists, antagonists, and fragments thereof, are useful for modulating intracellular Ca²⁺ levels, modulating Ca²⁺ sensitive signaling pathways, and modulating NFAT element associated signaling pathways.

In another embodiment of the present invention, an expression vector containing the complement of the polynucleotide encoding HGPRBMY4 polypeptide can be administered to an individual to treat or prevent a neoplastic disorder, including, but not limited to, the types of cancers and tumors described above.

In a further embodiment of the present invention, an expression vector containing the complement of the polynucleotide encoding HGPRBMY4 polypeptide can be administered to an individual to treat or prevent a neurological disorder, including, but not limited to, the types of disorders described above.

In yet another embodiment of the present invention, an expression vector containing the complement of the polynucleotide encoding HGPRBMY4 polypeptide can be administered to an individual to treat or prevent a prostate-related disorder, including, but not limited to, prostatitis, benign prostatic hyperplasia, prostate intraepithelial neoplasms, and urogenital cancers. Additionally, the present invention can be used to treat or prevent a colon-, breast-, or lung-related disease, disorder, or condition, including, but not limited to, Crohn's disease, Hirschsprung's disease, ulceritive colitis, prediverticular disease of the colon, colonic diverticulitis, colonic carcinoma, Hand-Schüller-Christian syndrome, eosinophilic granuloma, desquamative interstitial pneumonia, inflammatory bowel disease, breast cancer, endometrial cancer, ovarian cancer, Chagas' disease, bronchopulmonary dysplasia, post-inflammatory pseudotumor, Pancoast's syndrome, and other lung diseases, including carcinoma.

In another embodiment, the proteins, antagonists, antibodies, agonists, complementary sequences, or vectors of the present invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Antagonists or inhibitors of the HGPRBMY4 polypeptide of the present invention can be produced using methods which are generally known in the art. In fact, the HGPRBMY4 polypeptide has been shown to be involved in the regulation of mammalian NFkB and apoptosis pathways. Antagonists against HGPRBMY4 can therefore be desired for its therapeutic effect in relation to the E-selectin phenotype. The E-selectin promoter can be activated by NFkB. Elevated levels of cAMP can, however, inhibit TNF-alpha stimulation of E-selectin expression on endothelial cells (J. Biol. Chem., 1996, 271:20828; J. Biol. Chem., 1994, 269:19193). Based on this understanding of the regulation of E-selectin, genes that modulate E-selectin expression are also likely to be either in the NFkB pathway or regulate cellular cAMP levels. The utility for agonists and antagonists to the genes herein can either be simply based on modulation of E-selectin, or broader predictions can be made by the likelihood that these genes can have more global effects by possessing the ability to regulate the NFkB pathway and/or cAMP levels in human microvascular endothelial cells.

Antagonists and agonists, such as for example, HGPRBMY4, can be useful for reducing the expression of genes that control endothelial-leukocyte cell adhesion events and cytokine secretion (J. Mol. Cell. Cardiol., 2002, 34:349; Gene Ther., 2001, 8:1635; J. Clin. Invest., 1998, 101:1905; Blood, 1998, 92:3924; J. Immunol., 1991, 147:2777). Antagonists and agonists of HGPRBMY4 may block the binding of leukocytes and platelets to the endothelium, reducing inflammatory responses on the vessel walls, as well as, entry of leukocytes into tissues of autoimmune diseases, sites of inlammation, and in diseases such as chronic obstructive pulmonary disease (COPD), where foreign substances (i.e., smoke, allergens, environmental pollutants, and pathogens) drive immune cell recruitment and activation (Ann. Rev. Pharm. Toxicol., 2000, 40:283; Ann. Rev. Med., 1994, 45:361; Semin. Immunol., 1993, 5:237; Immunol. Today, 1993, 14:506, Clin. Cardiol. 1997, 20:822). Adhesion of metastatic cancer cells to the endothelium can also contribute to the metastatic process. Thus, antagonists or agonists can reduce endothelium-cancer cell interactions (Semin. Canc. Biol., 1993, 4:219; Clin. Exp. Metastasis., 1999, 17:183). Taken together data suggest that antisense to HGPRBMY4 can increase cAMP pools that act to stimulate IkB expression, which will drive down NFkB nuclear location. Under this scenario E-selectin expression would decrease when HGPRBMY4 is antagonized (either by antisense or small molecules) as a consequence of decrease in NFkB nuclear localization, as well as by increasing the cAMP pools.

Another embodiment of the invention involves a method of preventing, treating, or ameliorating an inflammatory or immune-related disease or disorder comprising inhibiting E-selectin expression by administering to a mammal in need thereof, HGPRBMY4 in an amount effective to inhibit E-selectin expression. Accordingly, E-selectin inhibition can result in one or more of the following: a) inhibition of E-selectin activity; b) inhibition of phosphorylation of IKB; c) inhibition of NFkB-dependent gene expression; or d) increase of cAMP pools. Inhibition of E-selectin is either directly or indirectly associated with the NFkB signaling pathway, such that inhibiting activation of NFkB-dependent gene expression associated with the inhibition of E-selectin expression, can be accomplished by administering to a mammal in need thereof an amount of HGPRBMY4 effective to inhibit E-selectin expression, thereby inhibiting activation of NFkB-dependent gene expression.

In a further embodiment, HGPRBMY4 transfected CHO-NFAT/CRE cell lines of the present invention are useful for the identification of agonists and antagonists of the HGPRBMY4 polypeptide. Representative uses of these cell lines would be their inclusion in a method of identifying HGPRBMY4 agonists and antagonists. Preferably, the cell lines are useful in a method for identifying a compound that modulates the biological activity of the HGPRBMY4 polypeptide, comprising the steps of (a) combining a candidate modulator compound with a host cell expressing the HGPRBMY4 polypeptide having the sequence as set forth in SEQ ID NO: 2; and (b) measuring an effect of the candidate modulator compound on the activity of the expressed HGPRBMY4 polypeptide. This method can also be used to identify candidate compounds that modulate E-selectin activity, where the candidate compound can be an agonist or antagonist of HGPRBMY4 activity. Antisense oligonucleotides can act as antagonists of HGPRBMY4 and E-selectin activity. Non-limiting antisense oligonucleotide sequences used for identifying an E-selectin/NFkB phenotype are described in Example 15. Representative vectors expressing the HGPRBMY4 polypeptide are referenced herein (for example, pcDNA3.1 hygro™) or otherwise known in the art.

The cell lines are also useful in a method of screening for a compounds that is capable of modulating the biological activity of HGPRBMY4 polypeptide, comprising the steps of: (a) determining the biological activity of the HGPRBMY4 polypeptide in the absence of a modulator compound; (b) contacting a host cell expression the HGPRBMY4 polypeptide with the modulator compound; and (c) determining the biological activity of the HGPRBMY4 polypeptide in the presence of the modulator compound; wherein a difference between the activity of the HGPRBMY4 polypeptide in the presence of the modulator compound and in the absence of the modulator compound indicates a modulating effect of the compound. Additional uses for these cell lines are described herein or otherwise known in the art. In particular, purified HGPRBMY4 protein, or fragments thereof, can be used to produce antibodies, or to screen libraries of pharmaceutical agents, to identify those which specifically bind HGPRBMY4.

Antibodies specific for HGPRBMY4 polypeptide, or immunogenic peptide fragments thereof, can be generated using methods that have long been known and conventionally practiced in the art. Such antibodies can include, but are not limited to, polyclonal, monoclonal, chimeric, single chain, Fab fragments, and fragments produced by an Fab expression library. Neutralizing antibodies, (i.e., those which inhibit dimer formation) are especially preferred for therapeutic use.

The present invention also encompasses the polypeptide sequences that intervene between each of the predicted HGPRBMY4 transmembrane domains. Since these regions are solvent accessible either extracellularly or intracellularly, they are particularly useful for designing antibodies specific to each region. Such antibodies can be useful as antagonists or agonists of the HGPRBMY4 full-length polypeptide and can modulate its activity.

The following serve as non-limiting examples of peptides or fragments that can be used to generate antibodies:

| | |
|---|---|
| MMVDPNGNESSATYFILIGLPGLEEAQ | (SEQ ID NO: 17) |
| RTEHSLHEPMY | (SEQ ID NO: 18) |
| NSTTIQFDACLLQM | (SEQ ID NO: 19) |
| HPLRHATVLTLPRVTK | (SEQ ID NO: 20) |
| KQLPFCRSNILSHSYCLHQDVMKLACDDIR | (SEQ ID NO: 21) |
| KTVLGLTREAQAKA | (SEQ ID NO: 22) |
| HRFSKRRDSP | (SEQ ID NO: 23) |
| KTKEIRQRILRLFHVATHASEP | (SEQ ID NO: 24) |

In preferred embodiments, the following N-terminal HGPRBMY4 N-terminal fragment deletion polypeptides are encompassed by the present invention: M1-Q27, M2-Q27, V3-Q27, D4-Q27, P5-Q27, N6-Q27, G7-Q27, N8-Q27, E9-Q27, S10-Q27, S11-Q27, A12-Q27, T13-Q27, Y14-Q27, F15-Q27, I16-Q27, L17-Q27, I18-Q27, G19-Q27, L20-Q27, and/or P21-Q27 of SEQ ID NO: 17. Polynucleotide sequences encoding these polypeptides are also provided. The present invention also encompasses the use of these N-terminal HGPRBMY4 N-terminal fragment deletion polypeptides as immunogenic and/or antigenic epitopes as described elsewhere herein.

In preferred embodiments, the following C-terminal HGPRBMY4 N-terminal fragment deletion polypeptides are encompassed by the present invention: M1-Q27, M1-A26, M1-E25, M1-E24, M1-L23, M1-G22, M1-P21, M1-L20, M1-G19, M1-I18, M1-L17, M1-I16, M1-F15, M1-Y14, M1-T13, M1-A12, M1-S11, M1-S10, M1-E9, M1-N8, and/or M1-G7 of SEQ ID NO: 17. Polynucleotide sequences encoding these polypeptides are also provided. The present invention also encompasses the use of these C-terminal HGPRBMY4 N-terminal fragment deletion polypeptides as immunogenic and/or antigenic epitopes as described elsewhere herein.

In preferred embodiments, the following N-terminal HGPRBMY4 TM1-2 intertransmembrane domain deletion polypeptides are encompassed by the present invention: R1-Y11, T2-Y11, E3-Y11, H4-Y11, and/or S5-Y11of SEQ ID NO: 18. Polynucleotide sequences encoding these polypeptides are also provided. The present invention also encompasses the use of these N-terminal HGPRBMY4 TM1-2 intertransmembrane domain deletion polypeptides as immunogenic and/or antigenic epitopes as described elsewhere herein.

In preferred embodiments, the following C-terminal HGPRBMY4 TM1-2 intertransmembrane domain deletion polypeptides are encompassed by the present invention: R1-Y11, R1-M10, R1-P9, R1-E8, and/or R1-H7 of SEQ ID NO: 18. Polynucleotide sequences encoding these polypeptides are also provided. The present invention also encompasses the use of these C-terminal HGPRBMY4 TM1-2 intertransmembrane domain deletion polypeptides as immunogenic and/or antigenic epitopes as described elsewhere herein.

In preferred embodiments, the following N-terminal HGPRBMY4 TM2-3 intertransmembrane domain deletion polypeptides are encompassed by the present invention: N1-M14, S2-M14, T3-M14, T4-M14, I5-M14, Q6-M14, F7-M14, and/or D8-M14 of SEQ ID NO: 19. Polynucleotide sequences encoding these polypeptides are also provided. The present invention also encompasses the use of these N-terminal HGPRBMY4 TM2-3 intertransmembrane domain deletion polypeptides as immunogenic and/or antigenic epitopes as described elsewhere herein.

In preferred embodiments, the following C-terminal HGPRBMY4 TM2-3 intertransmembrane domain deletion polypeptides are encompassed by the present invention: N1-M14, N1-Q13, N1-L12, N1-L11, N1-C10, N1-A9, N1-D8, and/or N1-F7 of SEQ ID NO: 19. Polynucleotide sequences encoding these polypeptides are also provided. The present invention also encompasses the use of these C-terminal HGPRBMY4 TM2-3 intertransmembrane domain deletion polypeptides as immunogenic and/or antigenic epitopes as described elsewhere herein.

In preferred embodiments, the following N-terminal HGPRBMY4 TM3-4 intertransmembrane domain deletion polypeptides are encompassed by the present invention: H1-K16, P2-K16, L3-K16, R4-K16, H5-K16, A6-K16, T7-K16, V8-K16, L9-K16, and/or T10-K16 of SEQ ID NO: 20. Polynucleotide sequences encoding these polypeptides are also provided. The present invention also encompasses the use of these N-terminal HGPRBMY4 TM3-4 intertransmembrane domain deletion polypeptides as immunogenic and/or antigenic epitopes as described elsewhere herein.

In preferred embodiments, the following C-terminal HGPRBMY4 TM3-4 intertransmembrane domain deletion polypeptides are encompassed by the present invention: H1-K16, H1-T15, H1-V14, H1-R13, H1-P12, H1-L11, H1-T10, H1-L9, H1-V8, and/or H1-T7 of SEQ ID NO: 20. Polynucleotide sequences encoding these polypeptides are also provided. The present invention also encompasses the use of these C-terminal HGPRBMY4 TM3-4 intertransmembrane domain deletion polypeptides as immunogenic and/or antigenic epitopes as described elsewhere herein.

In preferred embodiments, the following N-terminal HGPRBMY4 TM4-5 intertransmembrane domain deletion polypeptides are encompassed by the present invention: K1-R30, Q2-R30, L3-R30, P4-R30, F5-R30, C6-R30, R7-R30, S8-R30, N9-R30, I10-R30, L11-R30, S12-R30, H13-R30, S14-R30, Y15-R30, C16-R30, L17-R30, H18-R30, Q19-R30, D20-R30, V21-R30, M22-R30, K23-R30, and/or L24-R30 of SEQ ID NO: 21. Polynucleotide sequences encoding these polypeptides are also provided. The present invention also encompasses the use of these N-terminal HGPRBMY4 TM4-5 intertransmembrane domain deletion polypeptides as immunogenic and/or antigenic epitopes as described elsewhere herein.

In preferred embodiments, the following C-terminal HGPRBMY4 TM4-5 intertransmembrane domain deletion polypeptides are encompassed by the present invention: K1-R30, K1-129, K1-D28, K1-D27, K1-C26, K1-A25, K1-L24, K1-K23, K1-M22, K1-V21, K1-D20, K1-Q19, K1-H18, K1-L17, K1-C16, K1-Y15, K1-S14, K1-H13, K1-S12, K1-L11, K1-I10, K1-N9, K1-S8, and/or K1-R7 of SEQ ID NO: 21. Polynucleotide sequences encoding these polypeptides are also provided. The present invention also encompasses the use of these C-terminal HGPRBMY4 TM4-5 intertransmembrane domain deletion polypeptides as immunogenic and/or antigenic epitopes as described elsewhere herein.

In preferred embodiments, the following N-terminal HGPRBMY4 TM5-6 intertransmembrane domain deletion polypeptides are encompassed by the present invention: K1-A14, T2-A14, V3-A14, L4-A14, G5-A14, L6-A14, T7-A14, and/or R8-A14 of SEQ ID NO: 22. Polynucleotide sequences encoding these polypeptides are also provided. The present invention also encompasses the use of these N-terminal HGPRBMY4 TM5-6 intertransmembrane domain deletion polypeptides as immunogenic and/or antigenic epitopes as described elsewhere herein.

In preferred embodiments, the following C-terminal HGPRBMY4 TM5-6 intertransmembrane domain deletion polypeptides are encompassed by the present invention: K1-A14, K1-K13, K1-A12, K1-Q11, K1-A10, K1-E9, K1-R8, and/or K1-T7 of SEQ ID NO: 22. Polynucleotide sequences encoding these polypeptides are also provided. The present invention also encompasses the use of these C-terminal HGPRBMY4 TM5-6 intertransmembrane domain deletion polypeptides as immunogenic and/or antigenic epitopes as described elsewhere herein.

In preferred embodiments, the following N-terminal HGPRBMY4 TM6-7 intertransmembrane domain deletion polypeptides are encompassed by the present invention: H1-P10, R2-P10, F3-P10, and/or S4-P10 of SEQ ID NO: 23. Polynucleotide sequences encoding these polypeptides are also provided. The present invention also encompasses the use of these N-terminal HGPRBMY4 TM6-7 intertransmembrane domain deletion polypeptides as immunogenic and/or antigenic epitopes as described elsewhere herein.

In preferred embodiments, the following C-terminal HGPRBMY4 TM6-7 intertransmembrane domain deletion polypeptides are encompassed by the present invention: H1-P10, H1-S9, H1-D8, and/or H1-R7 of SEQ ID NO: 23. Polynucleotide sequences encoding these polypeptides are also provided. The present invention also encompasses the use of these C-terminal HGPRBMY4 TM6-7 intertransmembrane domain deletion polypeptides as immunogenic and/or antigenic epitopes as described elsewhere herein.

In preferred embodiments, the following N-terminal HGPRBMY4 C-terminal fragment deletion polypeptides are encompassed by the present invention: K1-P22, T2-P22, K3-P22, E4-P22, I5-P22, R6-P22, Q7-P22, R8-P22, I9-P22, L10-P22, R11-P22, L12-P22, F13-P22, H14-P22, V15-P22, and/or A16-P22 of SEQ ID NO: 24. Polynucleotide sequences encoding these polypeptides are also provided. The present invention also encompasses the use of these N-terminal HGPRBMY4 C-terminal fragment deletion polypeptides as immunogenic and/or antigenic epitopes as described elsewhere herein.

In preferred embodiments, the following C-terminal HGPRBMY4 C-terminal fragment deletion polypeptides are encompassed by the present invention: K1-P22, K1-E21, K1-S20, K1-A19, K1-H18, K1-T17, K1-A16, K1-V15, K1-H14, K1-F13, K1-L12, K1-R11, K1-L10, K1-I9, K1-R8, and/or K1-Q7 of SEQ ID NO: 24. Polynucleotide sequences encoding these polypeptides are also provided. The present invention also encompasses the use of these C-terminal HGPRBMY4 C-terminal fragment deletion polypeptides as immunogenic and/or antigenic epitopes as described elsewhere herein.

The HGPRBMY4 polypeptides of the present invention were determined to comprise several phosphorylation sites based upon the Motif algorithm (Genetics Computer Group, Inc.). The phosphorylation of such sites can regulate some biological activity of the HGPRBMY4 polypeptide. For example, phosphorylation at specific sites can be involved in regulating the proteins ability to associate or bind to other molecules (for example, proteins, ligands, substrates, DNA, etc.). In the present case, phosphorylation can modulate the ability of the HGPRBMY4 polypeptide to associate with other polypeptides, particularly cognate ligand for HGPRBMY4, or its ability to modulate certain cellular signal pathways.

The HGPRBMY4 polypeptide was predicted to comprise one PKC phosphorylation site using the Motif algorithm (Genetics Computer Group, Inc.). In vivo, protein kinase C exhibits a preference for the phosphorylation of serine or threonine residues. The PKC phosphorylation sites have the following consensus pattern: [ST]-x-[RK], where S or T represents the site of phosphorylation and ‘x’ an intervening amino acid residue. Additional information regarding PKC phosphorylation sites can be found in Woodget J. R., Gould K. L., Hunter T., Eur. J. Biochem. 161:177-184(1986), and Kishimoto A., Nishiyama K., Nakanishi H., Uratsuji Y., Nomura H., Takeyama Y., Nishizuka Y., J. Biol. Chem. 260:12492-12499(1985); which are hereby incorporated by reference herein.

In preferred embodiments, the following PKC phosphorylation site polypeptide is encompassed by the present invention: MVHRFSKRRDSPL (SEQ ID NO: 33). Polynucleotides encoding this polypeptide is also provided. The present invention also encompasses the use of the HGPRBMY4 PKC phosphorylation site polypeptide as an immunogenic and/or antigenic epitope as described elsewhere herein.

The HGPRBMY4 polypeptide was predicted to comprise three casein kinase II phosphorylation sites using the Motif algorithm (Genetics Computer Group, Inc.). Casein kinase II (CK-2) is a protein serine/threonine kinase whose activity is independent of cyclic nucleotides and calcium. CK-2 phosphorylates many different proteins. The substrate specificity [1] of this enzyme can be summarized as follows: (1) Under comparable conditions Ser is favored over Thr.; (2) An acidic residue (either Asp or Glu) must be present three residues from the C-terminal of the phosphate acceptor site; (3) Additional acidic residues in positions +1, +2, +4, and +5 increase the phosphorylation rate. Most physiological substrates have at least one acidic residue in these positions; (4) Asp is preferred to Glu as the provider of acidic determinants; and (5) A basic residue at the N-terminal of the acceptor site decreases the phosphorylation rate, while an acidic one will increase it.

A consensus pattern for casein kinase II phosphorylations site is as follows: [ST]-x(2)-[DE], wherein ‘x’ represents any amino acid, and S or T is the phosphorylation site.

Additional information specific to casein kinase II phosphorylation site domains can be found in reference to the following publication: Pinna L. A., Biochim. Biophys. Acta 1054:267-284(1990); which is hereby incorporated herein in its entirety.

In preferred embodiments, the following casein kinase II phosphorylation site polypeptide is encompassed by the present invention: VRTEHSLHEPMYTF (SEQ ID NO: 34), FLCMLSGIDILIST (SEQ ID NO: 35), and/or AIHSLSGMESTVLL (SEQ ID NO: 36). Polynucleotides encoding these polypeptides are also provided. The present invention also encompasses the use of this casein kinase II phosphorylation site polypeptide as an immunogenic and/or antigenic epitope as described elsewhere herein.

The HGPRBMY4 polypeptide was predicted to comprise two cAMP- and cGMP-dependent protein kinase phosphorylation site using the Motif algorithm (Genetics Computer Group, Inc.). There has been a number of studies relative to the specificity of cAMP- and cGMP-dependent protein kinases. Both types of kinases appear to share a preference for the phosphorylation of serine or threonine residues found close to at least two consecutive N-terminal basic residues.

A consensus pattern for cAMP- and cGMP-dependent protein kinase phosphorylation sites is as follows: [RK](2)-x-[ST], wherein “x” represents any amino acid, and S or T is the phosphorylation site.

Additional information specific to cAMP- and cGMP-dependent protein kinase phosphorylation sites can be found in reference to the following publication: Fremisco J. R., Glass D. B., Krebs E. G, J. Biol. Chem. 255:4240-4245(1980); Glass D. B., Smith S. B., J. Biol. Chem. 258:14797-14803(1983); and Glass D. B., El-Maghrabi M. R., Pilkis S. J., J. Biol. Chem. 261:2987-2993(1986); which is hereby incorporated herein in its entirety.

In preferred embodiments, the following cAMP- and cGMP-dependent protein kinase phosphorylation site polypeptide is encompassed by the present invention: HRFSKRRDSPLPVI (SEQ ID NO: 37). Polynucleotides encoding this polypeptide are also provided. The present invention also encompasses the use of this cAMP- and cGMP-dependent protein kinase phosphorylation site polypeptide as an immunogenic and/or antigenic epitope as described elsewhere herein.

The HGPRBMY4 polypeptide has been shown to comprise three glycosylation sites according to the Motif algorithm (Genetics Computer Group, Inc.). As discussed more specifically herein, protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion.

Asparagine glycosylation sites have the following concensus pattern, N-{P}-[ST]-{P}, wherein N represents the glycosylation site. However, it is well known that that potential N-glycosylation sites are specific to the consensus sequence Asn-Xaa-Ser/Thr. However, the presence of the consensus tripeptide is not sufficient to conclude that an asparagine residue is glycosylated, due to the fact that the folding of the protein plays an important role in the regulation of N-glycosylation. It has been shown that the presence of proline between Asn and Ser/Thr will inhibit N-glycosylation; this has been confirmed by a recent statistical analysis of glycosylation sites, which also shows that about 50% of the sites that have a proline C-terminal to Ser/Thr are not glycosylated. Additional information relating to asparagine glycosylation can be found in reference to the following publications, which are hereby incorporated by reference herein: Marshall R. D., Annu. Rev. Biochem. 41:673-702(1972); Pless D. D., Lennarz W. J., Proc. Natl. Acad. Sci. U.S.A. 74:134-138(1977); Bause E., Biochem. J. 209:331-336(1983); Gavel Y., von Heijne G., Protein Eng. 3:433-442(1990); and Miletich J. P., Broze G. J. Jr., J. Biol. Chem. 265:11397-11404 (1990).

In preferred embodiments, the following VDPNGNESSATYFI (SEQ ID NO: 38), IAVLGNLTIIYIVR (SEQ ID NO: 39), and/or AIFWFNSTTIQFDA (SEQ ID NO: 40). Polynucleotides encoding these polypeptides are also provided. The present invention also encompasses the use of these HGPRBMY4 asparagine glycosylation site polypeptide as inmmunogenic and/or antigenic epitopes as described elsewhere herein.

The HGPRBMY4 polypeptide was predicted to comprise four N-myristoylation sites using the Motif algorithm (Genetics Computer Group, Inc.). An appreciable number of eukaryotic proteins are acylated by the covalent addition of myristate (a C₁₄-saturated fatty acid) to their N-terminal residue via an amnide linkage. The sequence specificity of the enzyme responsible for this modification, myristoyl CoA:protein N-myristoyl transferase (NMT), has been derived from the sequence of known N-myristoylated proteins and from studies using synthetic peptides. The specificity seems to be the following: i) The N-terminal residue must be glycine; ii) In position 2, uncharged residues are allowed; iii) Charged residues, proline and large hydrophobic residues are not allowed; iv) In positions 3 and 4, most, if not all, residues are allowed; v) In position 5, small uncharged residues are allowed (Ala, Ser, Thr, Cys, Asn and Gly); serine is favored; and vi) In position 6, proline is not allowed.

A consensus pattern for N-myristoylation is as follows: G-{EDRKHPFYW}-x(2)-[STAGCN]-{P}, wherein ‘x’ represents any amino acid, and G is the N-myristoylation site.

Additional information specific to N-myristoylation sites can be found in reference to the following publication: Towler D. A., Gordon J. I., Adams S. P., Glaser L., Annu. Rev. Biochem. 57:69-99(1988); and Grand R. J. A., Biochem. J. 258:625-638(1989); which is hereby incorporated herein in its entirety.

In preferred embodiments, the following N-myristoylation site polypeptides are encompassed by the present invention: MVDPNGNESSATYFIL (SEQ ID NO: 41), LIGLPGLEEAQFWLAF (SEQ ID NO: 42), IHSLSGMESTVLLAMA (SEQ ID NO: 43), and/or QAKAFGTCVSHVCAVF (SEQ ID NO: 44). Polynucleotides encoding these polypeptides are also provided. The present invention also encompasses the use of these N-myristoylation site polypeptides as inmnunogenic and/or antigenic epitopes as described elsewhere herein.

Moreover, in confirmation of HGPRBMY4 representing a novel GPCR, the HGPRBMY4 polypeptide was predicted to comprise a G-protein coupled receptor motif using the Motif algorithm (Genetics Computer Group, Inc.). G-protein coupled receptors (also called R7G) are an extensive group of hormones, neurotransmitters, odorants and light receptors which transduce extracellular signals by interaction with guanine nucleotide-binding (G) proteins. Some examples of receptors that belong to this family are provided as follows: 5-hydroxytryptamine (serotonin) 1A to 1F, 2A to 2C, 4, 5A, 5B, 6 and 7, Acetylcholine, muscarinic-type, M1 to M5, Adenosine A1, A2A, A2B and A3, Adrenergic alpha-1A to -1C; alpha-2A to -2D; beta-1 to -3, Angiotensin II types I and II, Bombesin subtypes 3 and 4, Bradykinin B1 and B2, c3a and C5a anaphylatoxin, Cannabinoid CB1 and CB2, Chemokines C-C CC-CKR-1 to CC-CKR-8, Chemokines C-X-C CXC-CKR-1 to CXC-CKR-4, Cholecystokinin-A and cholecystokinin-B/gastrin, Dopamine D1 to D5, Endothelin ET-a and ET-b, fMet-Leu-Phe (fMLP) (N-formyl peptide), Follicle stimulating hormone (FSH-R), Galanin, Gastrin-releasing peptide (GRP-R), Gonadotropin-releasing hormone (GNRH-R), Histamine H1 and H2 (gastric receptor I), Lutropin-choriogonadotropic hormone (LSH-R), Melanocortin MC1R to MC5R, Melatonin, Neuromedin B (NMB-R), Neuromedin K (NK-3R), Neuropeptide Y types 1 to 6, Neurotensin (NT-R), Octopamine (tyramine) from insects, Odorants, Opioids delta-, kappa- and mu-types, Oxytocin (OT-R), Platelet activating factor (PAF-R), Prostacyclin, Prostaglandin D2, Prostaglandin E2, EP1 to EP4 subtypes, Prostaglandin F2, Purinoreceptors (ATP), Somatostatin types 1 to 5, Substance-K (NK-2R), Substance-P (NK-1R), Thrombin, Thromboxane A2, Thyrotropin (TSH-R), Thyrotropin releasing factor (TRH-R), Vasopressin V1a, V1b and V2, Visual pigments (opsins and rhodopsin), Proto-oncogene mas, Caenorhabditis elegans putative receptors C06G4.5, C38C10.1, C43C3.2, T27D1.3 and ZC84.4, Three putative receptors encoded in the genome of cytomegalovirus: US27, US28, and UL33., ECRF3, a putative receptor encoded in the genome of herpes virus saimiri.

The structure of all GPCRs are thought to be identical. They have seven hydrophobic regions, each of which most probably spans the membrane. The N-terminus is located on the extracellular side of the membrane and is often glycosylated, while the C-terminus is cytoplasmic and generally phosphorylated. Three extracellular loops alternate with three intracellular loops to link the seven transmembrane regions. Most, but not all of these receptors, lack a signal peptide. The most conserved parts of these proteins are the transmembrane regions and the first two cytoplasmic loops. A conserved acidic-Arg-aromatic triplet is present in the N-terminal extremity of the second cytoplasmic loop and could be implicated in the interaction with G proteins.

The putative consensus sequence for GPCRs comprises the conserved triplet and also spans the major part of the third transmembrane helix, and is as follows:

[GSTALIVMFYWC]-[GSTANCPDE]-{EDPKRH}-x(2)-[LIVMNQGA]-x(2)-[LWMFT]-[GSTANC]-[LIVMFYWSTAC]-[DENH]-R-[FYWCSH]-x(2)-[LIVM],

where “X” represents any amino acid.

Additional information relating to G-protein coupled receptors can be found in reference to the following publications: Strosberg A. D., Eur. J. Biochem. 196:1-10(1991); Kerlavage A. R., Curr. Opin. Struct. Biol. 1:394-401(1991); Probst W. C., Snyder L. A., Schuster D. I., Brosius J., Sealfon S. C., DNA Cell Biol. 11:1-20(1992); Savarese T. M., Fraser C. M., Biochem. J. 283:1-9(1992); Branchek T., Curr. Biol. 3:315-317(1993); Stiles G. L., J. Biol. Chem. 267:6451-6454(1992); Friell T., Kobilka B. K., Lefkowitz R. J., Caron M. G., Trends Neurosci. 11:321-324(1988); Stevens C. F., Curr. Biol. 1:20-22(1991); Sakurai T., Yanagisawa M., Masaki T., Trends Pharmacol. Sci. 13:103-107(1992); Salesse R., Remy J. J., Levin J. M., Jallal B., Garnier J., Biochimie 73:109-120(1991); Lancet D., Ben-Arie N., Curr. Biol. 3:668-674(1993); Uhl G. R., Childers S., Pasternak G., Trends Neurosci. 17:89-93(1994); Barnard E. A., Burnstock G., Webb T. E., Trends Pharmacol. Sci. 15:67-70(1994); Applebury M. L., Hargrave P. A., Vision Res. 26:1881-1895(1986); Attwood T. K., Eliopoulos E. E., Findlay J. B. C., Gene 98:153-159(1991); Hiyper Text Transfer Protocol://World Wide Web.gcrdb.University of Texas Health Science Center at San Antonio.educational organization; and Hyper Text Transfer Protocol://swift.European Molecular Biology Laboratory-heidelberg.Deutschland/7tm/.

In preferred embodiments, the following G-protein coupled receptors signature polypeptide is encompassed by the present invention: HSLSGMESTVLLAMAFDRYVAICHPLR (SEQ ID NO: 45). Polynucleotides encoding this polypeptide is also provided. The present invention also encompasses the use of the HGPRBMY4 G-protein coupled receptors signature polypeptide as immunogenic and/or antigenic epitopes as described elsewhere herein.

For the production of antibodies, various hosts including goats, rabbits, sheep, rats, mice, humans, and others, can be immunized by injection with the HGPRBMY4 polypeptide, or any fragment or oligopeptide thereof, which has immunogenic properties. Depending on the host species, various adjuvants can be used to increase the immunological response. Non-limiting examples of suitable adjuvants include Freund's (incomplete), mineral gels such as aluminum hydroxide or silica, and surface active substances such as lysolecithin, pluronic polyols, polyanions, peptides, oil emulsions, KLH, and dinitrophenol. Adjuvants typically used in humans include BCG (bacilli Calmette Guérin) and Corynebacterium parvumn.

Preferably, the peptides, fragments, or oligopeptides used to induce antibodies to HGPRBMY4 polypeptide (i.e., immunogens) have an amino acid sequence having at least five amino acids, and more preferably, at least 7-10 amino acids. It is also preferable that the immunogens are identical to a portion of the amino acid sequence of the natural protein; they can also contain the entire amino acid sequence of a small, naturally occurring molecule. The peptides, fragments or oligopeptides can comprise a single epitope or antigenic determinant or multiple epitopes. Short stretches of HGPRBMY4 amino acids can be fused with those of another protein, such as KLH, and antibodies are produced against the chimeric molecule.

Monoclonal antibodies to the HGPRBMY4 polypeptide, or immunogenic fragments thereof, can be prepared using any technique which provides for the production of antibody molecules by continuous cell lines in culture. These include, but are not limited to, the hybridoma technique, the human B-cell hybridoma technique, and the EBV-hybridoma technique (G. Kohler et al., 1975, Nature, 256:495-497; D. Kozbor et al., 1985, J. Immunol. Methods, 81:31-42; R. J. Cote et al., 1983, Proc. Natl. Acad. Sci. USA, 80:2026-2030; and S. P. Cole et al., 1984, Mol. Cell Biol., 62:109-120). The production of monoclonal antibodies is well known and routinely used in the art.

In addition, techniques developed for the production of “chimeric antibodies,” the splicing of mouse antibody genes to human antibody genes to obtain a molecule with appropriate antigen specificity and biological activity can be used (S. L. Morrison et al., 1984, Proc. Natl. Acad. Sci. USA, 81:6851-6855; M. S. Neuberger et al., 1984, Nature, 312:604-608; and S. Takeda et al., 1985, Nature, 314:452-454). Alternatively, techniques described for the production of single chain antibodies can be adapted, using methods known in the art, to produce the HGPRBMY4 polypeptide-specific single chain antibodies. Antibodies with related specificity, but of distinct idiotypic composition, can be generated by chain shuffling from random combinatorial immunoglobulin libraries (D. R. Burton, 1991, Proc. Natl. Acad. Sci. USA, 88:11120-3). Antibodies can also be produced by inducing in vivo production in the lymphocyte population or by screening recombinant immunoglobulin libraries or panels of highly specific binding reagents as disclosed in the literature (R. Orlandi et al., 1989, Proc. Natl. Acad. Sci. USA, 86:3833-3837 and G. Winter et al., 1991, Nature, 349:293-299).

Antibody fragments which contain specific binding sites for HGPRBMY4 polypeptide can also be generated. For example, such fragments include, but are not limited to, F(ab′)2 fragments which can be produced by pepsin digestion of the antibody molecule and Fab fragments which can be generated by reducing the disulfide bridges of the F(ab′)2 fragments. Alternatively, Fab expression libraries can be constructed to allow rapid and easy identification of monoclonal Fab fragments with the desired specificity (W. D. Huse et al., 1989, Science, 254.1275-1281).

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve measuring the formation of complexes between the HGPRBMY4 polypeptide and its specific antibody. A two-site, monoclonal-based immunoassay utilizing monoclonal antibodies reactive with two non-interfering HGPRBMY4 polypeptide epitopes is preferred, but a competitive binding assay can also be employed (Maddox, supra).

Another aspect of the invention relates to a method for inducing an immunological response in a mammal which comprises inoculating the mammal with HGPRBMY4 polypeptide, or a fragment thereof, adequate to produce antibody and/or T cell immune response to protect said animal from infections such as bacterial, fungal, protozoan and viral infections, particularly infections caused by HIV-1 or HIV-2. Yet another aspect of the invention relates to a method of inducing immunological response in a mammal which comprises, delivering HGPRBMY4 polypeptide via a vector directing expression of HGPRBMY4 polynucleotide in vivo in order to induce such an immunological response to produce antibody to protect said animal from diseases.

A further aspect of the invention relates to an immunological/vaccine formulation (composition) which, when introduced into a mammalian host, induces an immunological response in that mammal to an HGPRBMY4 polypeptide wherein the composition comprises a HGPRBMY4 polypeptide or HGPRBMY4 gene. The vaccine formulation can further comprise a suitable carrier. Since the HGPRBMY4 polypeptide can be broken down in the stomach, it is preferably administered parenterally (including subcutaneous, intramuscular, intravenous, intradermal, etc. injection). Formulations suitable for parenteral administration include aqueous and non-aqueous sterile injection solutions which can contain anti-oxidants, buffers, bacteriostats and solutes which render the formulation isotonic with the blood of the recipient; and aqueous and non-aqueous sterile suspensions which can include suspending agents or thickening agents. The formulations can be presented in unit-dose or multi-dose containers, for example, sealed ampoules and vials, and can be stored in a freeze-dried condition requiring only the addition of the sterile liquid carrier immediately prior to use. The vaccine formulation can also include adjuvant systems for enhancing the immunogenicity of the formulation, such as oil-in-water systems and other systems known in the art. The dosage will depend on the specific activity of the vaccine and can be readily determined by routine experimentation.

In an embodiment of the present invention, the polynucleotide encoding the HGPRBMY4 polypeptide, or any fragment or complement thereof, can be used for therapeutic purposes. In one aspect, antisense, to the polynucleotide encoding the HGPRBMY4 polypeptide, can be used in situations in which it would be desirable to block the transcription of the mRNA. In particular, cells can be transformed with sequences complementary to polynucleotides encoding HGPRBMY4 polypeptide. Thus, complementary molecules can be used to modulate HGPRBMY4 polynucleotide and polypeptide activity, or to achieve regulation of gene function. Such technology is now well known in the art, and sense or antisense oligomers or oligonucleotides, or larger fragments, can be designed from various locations along the coding or control regions of polynucleotide sequences encoding HGPRBMY4 polypeptide.

Expression vectors derived from retroviruses, adenovirus, herpes or vaccinia viruses, or from various bacterial plasmids can be used for delivery of nucleotide sequences to the targeted organ, tissue or cell population. Methods, which are well known to those skilled in the art, can be used to construct recombinant vectors which will express a nucleic acid sequence that is complementary to the nucleic acid sequence encoding the HGPRBMY4 polypeptide. These techniques are described both in J. Sambrook et al., supra and in F. M. Ausubel et al., supra.

Polypeptides used in treatment can also be generated endogenously in the subject, in treatment modalities often referred to as “gene therapy.” Thus for example, cells from a subject can be engineered with a polynucleotide, such as DNA or RNA, to encode a polypeptide ex vivo, and for example, by the use of a retroviral plasmid vector. The cells can then be introduced into the subject.

The genes encoding the HGPRBMY4 polypeptide can be turned off by transforming a cell or tissue with an expression vector that expresses high levels of an HGPRBMY4 polypeptide-encoding polynucleotide, or a fragment thereof. Such constructs can be used to introduce untranslatable sense or antisense sequences into a cell. Even in the absence of integration into the DNA, such vectors can continue to transcribe RNA molecules until they are disabled by endogenous nucleases. Transient expression can last for a month or more with a non-replicating vector, and even longer if appropriate replication elements are designed to be part of the vector system.

Modifications of gene expression can be obtained by designing antisense molecules or complementary nucleic acid sequences (DNA, RNA, or PNA), to the control, 5′, or regulatory regions of the gene encoding the HGPRBMY4 polypeptide, (e.g., signal sequence, promoters, enhancers, and introns). Oligonucleotides derived from the transcription initiation site, for example, between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described (see, for example, J. E. Gee et al., 1994, In: B. E. Huber and B. I. Carr, Molecular and Immunologic Approaches, Futura Publishing Co., Mt. Kisco, N.Y.). The antisense molecule or complementary sequence can also be designed to block translation of mRNA by preventing the transcript from binding to ribosomes.

Ribozymes, i.e., enzymatic RNA molecules, can also be used to catalyze the specific cleavage of RNA. The mechanism of ribozyme action involves sequence-specific hybridization of the ribozyme molecule to complementary target RNA, followed by endonucleolytic cleavage. Suitable examples include engineered hammerhead motif ribozyme molecules that can specifically and efficiently catalyze endonucleolytic cleavage of sequences encoding HGPRBMY4 polypeptide.

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides of the region of the target gene containing the cleavage site can be evaluated for secondary structural features which can render the oligonucleotide inoperable. The suitability of candidate targets can also be evaluated by testing accessibility to hybridization with complementary oligonucleotides using ribonuclease protection assays.

Complementary ribonucleic acid molecules and ribozymes according to the invention can be prepared by any method known in the art for the synthesis of nucleic acid molecules. Such methods include techniques for chemically synthesizing oligonucleotides, for example, solid phase phosphoramidite chemical synthesis. Alternatively, RNA molecules can be generated by in vitro and in vivo transcription of DNA sequences encoding HGPRBMY4. Such DNA sequences can be incorporated into a wide variety of vectors with suitable RNA polymerase promoters such as T7 or SP. Alternatively, the cDNA constructs that constitutively or inducibly synthesize complementary RNA can be introduced into cell lines, cells, or tissues.

RNA molecules can be modified to increase intracellular stability and half-life. Possible modifications include, but are not limited to, the addition of flanking sequences at the 5′ and/or 3′ ends of the molecule, or the use of phosphorothioate or 2′ O-methyl, rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytosine, guanine, thymine, and uridine which are not as easily recognized by endogenous endonucleases.

Many methods for introducing vectors into cells or tissues are available and are equally suitable for use in vivo, in vitro, and ex vivo. For ex vivo therapy, vectors can be introduced into stem cells taken from the patient and clonally propagated for autologous transplant back into that same patient. Delivery by transfection and by liposome injections can be achieved using methods, which are well known in the art.

Any of the therapeutic methods described above can be applied to any individual in need of such therapy, including, for example, mammals such as dogs, cats, cows, horses, rabbits, monkeys, and most preferably, humans.

A further embodiment of the present invention embraces the administration of a pharmaceutical composition, in conjunction with a pharmaceutically acceptable carrier, diluent, or excipient, for any of the above-described therapeutic uses and effects. Such pharmaceutical compositions can comprise HGPRBMY4 nucleic acid, polypeptide, or peptides, antibodies to HGPRBMY4 polypeptide, mimetics, agonists, antagonists, or inhibitors of HGPRBMY4 polypeptide or polynucleotide. The compositions can be administered alone or in combination with at least one other agent, such as a stabilizing compound, which can be administered in any sterile, biocompatible pharmaceutical carrier, including, but not limited to, saline, buffered saline, dextrose, and water. The compositions can be administered to a patient alone, or in combination with other agents, drugs, hormones, or biological response modifiers.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

In addition to the active ingredients (i.e., the HGPRBMY4 nucleic acid or polypeptide, or functional fragments thereof), the pharmaceutical compositions can contain suitable pharmaceutically acceptable carriers or excipients comprising auxiliaries which facilitate processing of the active compounds into preparations which can be used pharmaceutically. Further details on techniques for formulation and administration are provided in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained by the combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents can be added, such as cross-linked polyvinyl pyrrolidone, agar, alginic acid, or a physiologically acceptable salt thereof, such as sodium alginate.

Dragee cores can be used in conjunction with physiologically suitable coatings, such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification, or to characterize the quantity of active compound, i.e., dosage.

Pharmaceutical preparations, which can be used orally, include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances, which increase the viscosity of the suspension, such as sodium carboxymethyl cellulose, sorbitol, or dextran. In addition, suspensions of the active compounds can be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension can also contain suitable stabilizers or agents which increase the solubility of the compounds to allow for the preparation of highly concentrated solutions.

For topical or nasal administration, penetrants or permeation agents that are appropriate to the particular barrier to be permeated are used in the formulation. Such penetrants are generally known in the art.

The pharmaceutical compositions of the present invention can be manufactured in a manner that is known in the art, such as but not limited by means of conventional mixing, dissolving, granulating, dragee-making, levigating, emulsifying, encapsulating, entrapping, or lyophilizing processes.

The pharmaceutical composition can be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation can be a lyophilized powder which can contain any or all of the following: 1-50 mM histidine, 0.1%-2% sucrose, and 2%-7% mannitol, at a pH range of 4.5 to 5.5, combined with a buffer prior to use. After the pharmaceutical compositions have been prepared, they can be placed in an appropriate container and labeled for treatment of an indicated condition. For administration of HGPRBMY4 product, such labeling would include amount, frequency, and method of administration.

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, for example, using neoplastic cells, or in animal models, usually mice, rabbits, dogs, or pigs. The animal model can also be used to determine the appropriate concentration range and route of administration. Such information can then be used and extrapolated to determine useful doses and routes for administration in humans.

A therapeutically effective dose refers to that amount of active ingredient, for example, HGPRBMY4 polypeptide, or fragments thereof, antibodies to HGPRBMY4 polypeptide, agonists, antagonists or inhibitors of HGPRBMY4 polypeptide, which ameliorates, reduces, or eliminates the symptoms or condition. Therapeutic efficacy and toxicity can be determined by standard pharmaceutical procedures in cell cultures or experimental animals, for example, ED₅₀ (the dose therapeutically effective in 50% of the population) and LD₅₀ (the dose lethal to 50% of the population). The dose ratio of toxic to therapeutic effects is the therapeutic index, which can be expressed as the ratio, ED₅₀/LD₅₀. Pharmaceutical compositions which exhibit large therapeutic indices are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, sensitivity of the patient, and the route of administration.

The practitioner, who will consider the factors related to the individual requiring treatment, will determine the exact dosage. Dosage and administration are adjusted to provide sufficient levels of the active moiety or to maintain the desired effect. Factors, which can be taken into account, include the severity of the individual's disease state, general health of the patient, age, weight, and gender of the patient, diet, time and frequency of administration, drug combination(s), reaction sensitivities, and tolerance/response to therapy. As a general guide, long-acting pharmaceutical compositions can be administered every 3 to 4 days, every week, or once every two weeks, depending on half-life and clearance rate of the particular formulation. Variations in these dosage levels can be adjusted using standard empirical routines for optimization, as is well understood in the art.

Normal dosage amounts can vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will employ different formulations for nucleotides than for proteins or their inhibitors. Similarly, delivery of polynucleotides or polypeptides will be specific to particular cells, conditions, locations, and the like.

In another embodiment of the present invention, antibodies which specifically bind to the HGPRBMY4 polypeptide can be used for the diagnosis of conditions or diseases characterized by expression (or overexpression) of the HGPRBMY4 polynucleotide or polypeptide, or in assays to monitor patients being treated with the HGPRBMY4 polypeptide, or its agonists, antagonists, or inhibitors. The antibodies useful for diagnostic purposes can be prepared in the same manner as those described herein for use in therapeutic methods. Diagnostic assays for the HGPRBMY4 polypeptide include methods which utilize the antibody and a label to detect the protein in human body fluids or extracts of cells or tissues. The antibodies can be used with or without modification, and can be labeled by joining them, either covalently or non-covalently, with a reporter molecule. A wide variety of reporter molecules, which are known in the art, can be used, several of which are described above.

The use of mammalian cell reporter assays to demonstrate functional coupling of known GPCRs (G Protein Coupled Receptors) has been well documented in the literature (Gilman, 1987, Boss et al., 1996; Alam & Cook, 1990; George et al., 1997; Selbie & Hill, 1998; Rees et al., 1999). In fact, reporter assays have been successfully used for identifying novel small molecule agonists or antagonists against GPCRs as a class of drug targets (Zlokarnik et al., 1998; George et al., 1997; Boss et al., 1996; Rees et al, J Biomol Screen, 6(1):19-27 (2001)). In such reporter assays, a promoter is regulated as a direct consequence of activation of specific signal transduction cascades following agonist binding to a GPCR (Alam & Cook 1990; Selbie & Hill, 1998; Boss et al., 1996; George et al., 1997; Gilman, 1987).

A number of response element-based reporter systems have been developed that enable the study of GPCR function. These include cAMP response element (CRE)-based reporter genes for G alpha i/o, G alpha s-coupled GPCRs, Nuclear Factor Activator of Transcription (NFAT)-based reporters for G alpha q/11 or the promiscuous G protein G alpha 15/16-coupled receptors and MAP kinase reporter genes for use in Galpha i/o coupled receptors (Selbie & Hill, 1998; Boss et al., 1996; George et al., 1997; Blahos, et al., 2001; Offermann & Simon, 1995; Gilman, 1987; Rees et al., 2001). Transcriptional response elements that regulate the expression of Beta-Lactamase within a CHO K1 cell line (CHO/NFAT-CRE: Aurora Biosciences™) (Zlokarnik et al., 1998) have been implemented to characterize the function of the orphan HGPRBMY4 polypeptide of the present invention. The system enables demonstration of constitutive G-protein coupling to endogenous cellular signaling components upon intracellular overexpression of orphan receptors. Overexpression has been shown to represent a physiologically relevant event. For example, it has been shown that overexpression occurs in nature during metastatic carcinomas, wherein defective expression of the monocyte chemotactic protein 1 receptor, CCR2, in macrophages is associated with the incidence of human ovarian carcinoma (Sica, et al., 2000; Salcedo et al., 2000). Indeed, it has been shown that overproduction of the Beta 2 Adrenergic Receptor in transgenic mice leads to constitutive activation of the receptor signaling pathway such that these mice exhibit increased cardiac output (Kypson et al., 1999; Dorn et al., 1999). These are only a few of the many examples demonstrating constitutive activation of GPCRs whereby many of these receptors are likely to be in the active, R*, conformation (J. Wess, 1997) (see Example 11).

Several assay protocols including ELISA, RIA, and FACS for measuring the HGPRBMY4 polypeptide are known in the art and provide a basis for diagnosing altered or abnormal levels of HGPRBMY4 polypeptide expression. Normal or standard values for HGPRBMY4 polypeptide expression are established by combining body fluids or cell extracts taken from normal mammalian subjects, preferably human, with antibody to the HGPRBMY4 polypeptide under conditions suitable for complex formation. The amount of standard complex formation can be quantified by various methods; photometric means are preferred. Quantities of HGPRBMY4 polypeptide expressed in subject sample, control sample, and disease samples from biopsied tissues are compared with the standard values. Deviation between standard and subject values establishes the parameters for diagnosing disease.

### Microarrays and Screening Assays

In another embodiment of the present invention, oligonucleotides, or longer fragments derived from the HGPRBMY4 polynucleotide sequence described herein can be used as targets in a microarray. The microarray can be used to monitor the expression level of large numbers of genes simultaneously (to produce a transcript image), and to identify genetic variants, mutations and polymorphisms. This information can be used to determine gene function, to understand the genetic basis of a disease, to diagnose disease, and to develop and monitor the activities of therapeutic agents. In a particular aspect, the microarray is prepared and used according to the methods described in WO 95/11995 (Chee et al.); D. J. Lockhart et al., 1996, Nature Biotechnology, 14:1675-1680; and M. Schena et al., 1996, Proc. Natl. Acad. Sci. USA, 93:10614-10619. Microarrays are further described in U.S. Pat. No. 6,015,702 to P. Lal et al.

In another embodiment of this invention, the nucleic acid sequence, which encodes the HGPRBMY4 polypeptide, can also be used to generate hybridization probes, which are useful for mapping the naturally occurring genomic sequence. The sequences can be mapped to a particular chromosome, to a specific region of a chromosome, or to artificial chromosome constructions (HACs), yeast artificial chromosomes (YACs), bacterial artificial chromosomes (BACs), bacterial PI constructions, or single chromosome cDNA libraries, as reviewed by C. M. Price, 1993, Blood Rev., 7:127-134 and by B. J. Trask, 1991, Trends Genet., 7:149-154.

Fluorescent In Situ Hybridization (FISH), (as described in I. Verma et al., 1988, Human Chromosomes: A Manual of Basic Techniques, Pergamon Press, New York, N.Y.) can be correlated with other physical chromosome mapping techniques and genetic map data. Examples of genetic map data can be found in numerous scientific journals or at Online Mendelian Inheritance in Man (OMIM). Correlation between the location of the gene encoding the HGPRBMY4 polypeptide on a physical chromosomal map and a specific disease, or predisposition to a specific disease, can help delimit the region of DNA associated with that genetic disease. The nucleotide sequences, particularly that of SEQ ID NO: 1, or fragments thereof, according to this invention can be used to detect differences in gene sequences between normal, carrier, or affected individuals.

In situ hybridization of chromosomal preparations and physical mapping techniques such as linkage analysis using established chromosomal markers can be used for extending genetic maps. Often the placement of a gene on the chromosome of another mammalian species, such as mouse, can reveal associated markers, even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (R. A. Gatti et al., 1988, Nature, 336:577-580), any sequences mapping to that area can represent associated or regulatory genes for further investigation. The nucleotide sequence of the present invention can also be used to detect differences in the chromosomal location due to translocation, inversion, and the like, among normal, carrier, or affected individuals.

In another embodiment of the present invention, the HGPRBMY4 polypeptide, its catalytic or immunogenic fragments or oligopeptides thereof, can be used for screening libraries of compounds in any of a variety of drug screening techniques. The fragment employed in such screening can be free in solution, affixed to a solid support, borne on a cell surface, or located intracellularly. The formation of binding complexes, between HGPRBMY4 polypeptide, or portion thereof, and the agent being tested, can be measured utilizing techniques commonly practiced in the art.

Another technique for drug screening which can be used provides for high throughput screening of compounds having suitable binding affinity to the protein of interest as described in WO 84/03564 (Venton, et al.). In this method, as applied to the HGPRBMY4 protein, large numbers of different small test compounds are synthesized on a solid substrate, such as plastic pins or some other surface. The test compounds are reacted with the HGPRBMY4 polypeptide, or fragments thereof, and washed. Bound HGPRBMY4 polypeptide is then detected by methods well known in the art. Purified HGPRBMY4 polypeptide can also be coated directly onto plates for use in the aforementioned drug screening techniques. Alternatively, non-neutralizing antibodies can be used to capture the peptide and immobilize it on a solid support.

In a further embodiment of this invention, competitive drug screening assays can be used in which neutralizing antibodies, capable of binding the HGPRBMY4 polypeptide, specifically compete with a test compound for binding to the HGPRBMY4 polypeptide. In this manner, the antibodies can be used to detect the presence of any peptide, which shares one or more antigenic determinants with the HGPRBMY4 polypeptide.

### EXAMPLES

The Examples herein are meant to exemplify the various aspects of carrying out the invention and are not intended to limit the scope of the invention in any way. The Examples do not include detailed descriptions for conventional methods employed, such as in the construction of vectors, the insertion of cDNA into such vectors, or the introduction of the resulting vectors into the appropriate host. Such methods are well known to those skilled in the art and are described in numerous publication's, for example, Sambrook, Fritsch, and Maniatis, Molecular Cloning: a Laboratory Manual, 2^{nd} Edition, Cold Spring Harbor Laboratory Press, USA, (1989).

### Example 1

### Bioinformatics Analysis

G-protein coupled receptor sequences were used as a probe to search the Incyte and public domain EST databases. The search program used was gapped BLAST (S. F. Altschul, et al., Nuc. Acids Res., 25:3389-4302 (1997)). The top EST hits from the BLAST results were searched back against the non-redundant protein and patent sequence databases. From this analysis, ESTs encoding potential novel GPCRs were identified based on sequence homology. The Incyte EST (CloneID:998550) was selected as potential novel GPCR candidate, called HGPRBMY4, for subsequent analysis. This EST was sequenced and the full-length clone of this GPCR was obtained using the EST sequence information and conventional methods. The complete protein sequence of HGPRBMY4 was analyzed for potential transmembrane domains. The TMPRED program (K. Hofmann and W. Stoffel, Biol. Chem., 347:166 (1993)) was used for transmembrane prediction. The program predicted seven transmembrane domains and the predicted domains match with the predicated transmembrane domains of related GPCRs at the sequence level. Based on sequence, structure and known GPCR signature sequences, the orphan protein, HGPRBMY4, is a novel human GPCR.

### Example 2

### Cloning of the Novel Human GPCR HGPRBMY4

Using the EST sequence, an antisense 80 base pair oligonucleotide with biotin on the 5′ end was designed that was complementary to the putative coding region of HGPRBMY4 as follows: 5′-b-GATCCACCATCATGAAGAAGCTGAAC TGTGACCAGCACCAGGCAGGTAGAGGCTCAACCGTATGGAAGGAATGTGT GACC-3′ (SEQ ID NO: 5). This biotinylated oligo was incubated with a mixture of single-stranded covalently closed circular cDNA libraries, which contained DNA of the sense strand. Hybrids between the biotinylated oligo and the circular cDNA were captured on streptavidin magnetic beads. Upon thermal release of the cDNA from the biotinylated oligo, the single stranded cDNA was converted into double strands using a primer homologous to a sequence on the cDNA cloning vector. The double stranded cDNA was introduced into E. coli by electroporation and the resulting colonies were screened by PCR, using a primer pair designed from the EST sequence to identify the proper cDNA.

Oligos used to identify the cDNA by PCR were as follows:

HGPRBMY4s (SEQ ID NO: 6) 5′-ACTGAGCACAGCCTGCATGA-3′; and

HGPRBMY4a (SEQ ID NO: 7) 5′-b-TCTGTAGCAGACAAGCATCAAACTG -3′

Those cDNA clones that were positive by PCR had the inserts sized and two of the largest clones (4.5 Kb and 3.3 Kb) were chosen for DNA sequencing. Both clones had identical sequence over the common regions.

### Example 3

### Expression Profiling of Novel Human GPCR, HGPRBMY4

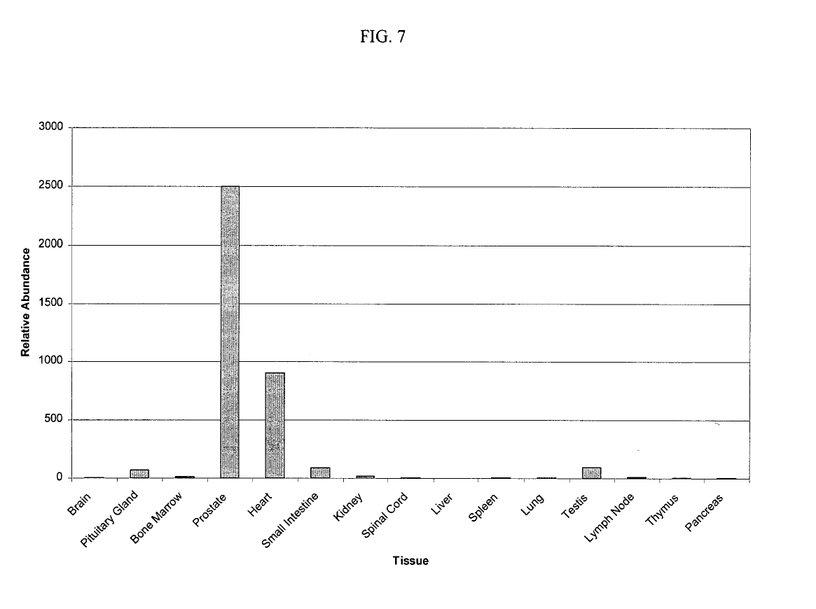

The same PCR primer pair used to identify HGPRBMY4 cDNA clones (HGPRBMY4s-SEQ ID NO: 6 and HGPRBMY4a-SEQ ID NO: 7) was used to measure the steady state levels of mRNA by quantitative PCR. Briefly, first strand cDNA was made from commercially available mRNA. The relative amount of cDNA used in each assay was determined by performing a parallel experiment using a primer pair for the cyclophilin gene, which is expressed in equal amounts in all tissues. The cyclophilin primer pair detected small variations in the amount of cDNA in each sample, and these data were used for normalization of the data obtained with the primer pair for HGPRBMY4. The PCR data were converted into a relative assessment of the difference in transcript abundance among the tissues tested and the data are presented in FIG. 7. Transcripts of the orphan GPCR, HGPRBMY4, were found to be highly expressed in prostate and moderately in heart.

### Example 4

### G-protein Coupled Receptor Immunohistochemistry Hybridization Expression Profiling

Immunohistochemistry expression using the LifeSpan database, describes positive staining in normal, benign, and carcinoma cells . Slides containing paraffin sections (LifeSpan BioSciences, Inc.; Seattle, Wash.) were deparaffinized through xylene and alcohol, rehydrated, and then subjected to the steam method of target retrieval (#S1700; DAKO Corp.; Carpenteria, Calif.).

Immunohistochemical assay techniques are commonly known in the art and are described briefly herein. Immunocytochemical (ICC) experiments were performed on a DAKO autostainer following the procedures and reagents developed by DAKO. Specifically, the slides were blocked with avidin, rinsed, blocked with biotin, rinsed, protein blocked with DAKO universal protein block, machine blown dry, primary antibody, incubated, and the slides rinsed. Biotinylated secondary antibody was applied using the manufacturer's instructions (1 drop/10 ml, or approximately 0.75 g/mL), incubated, rinsed slides, and applied Vectastain ABC-AP reagent for 30 minutes. Vector Red was used as substrate and prepared according to the manufacturer's instructions just prior to use.

Moderate to strong positivity was identified in the small subsets of normal prostatic epithelial cells, with most cells staining faintly (five of five samples). A small subset of glands was negative. Most staining was noted near the luminal ends of epithelial cells, whereas the basal cells were predominantly negative with a small subset showing blush to faint staining. Smooth muscle stromal myocytes were predominantly negative with a small subset showing blush to faint staining.

In samples of glandular and stromal hyperplasia, prostatic epithelial cells stained predominantly faintly with small subsets showing moderate staining (three of three samples). Interestingly, basal cells in hyperplastic glands appeared to have increased staining compared to basal cells in normal glands (three of three samples). These basal cells within hyperplastic glands showed predominantly moderate staining, with small subsets of blush to faint staining cells. Dysplastic regions did not appear to stain differently than non-dysplastic regions. Staining was generally limited to the luminal ends of the epithelial cells, although subsets of cells stained more uniformly.

Prostatic adenocarcinoma cells were present in Gleason pattern 3 (moderately differentiated) and 4 (moderately to poorly differentiated). Malignant cells in pattern 3 stained faintly, and subsets were negative or showed blush staining, or stained moderately to strongly (five of five samples). Occasional small malignant glands stained strongly. The staining pattern showed predominantly uniform distribution throughout the cells. The malignant cells of Gleason pattern 4 were more frequently negative than pattern 3 cells, with small subsets of cells staining blush to strongly (two of two samples with pattern 4).

Moderate to strong staining was also observed in the epithelium lining Bowman's capsule, subsets of the smooth muscle cells in the muscularis propria of the small intestine and arrector pili of skin, subsets of vascular smooth muscle cells and collecting duct cells in the renal medulla, rare subsets of hepatocytes (most were negative or showed blush staining), subsets of type II pneumocytes adjacent to alveolar hemorrhage, pleural mesothelial cells, subsets of skeletal muscle myocytes, and subsets of sebocytes in dermis.

Blush to faint staining was identified in small subsets of each of the following cell types: neurons, astrocytes, cardiac myocytes, capillary endothelial cells, plasma cells, smooth muscle cells, hepatocytes, proximal and distal renal tubules and collecting ducts, type I pneumocytes, macrophages, skeletal muscle myocytes, splenic lymphocytes, and pancreatic islet cells.

The majority of the following cell types were negative: hepatocytes, bile duct cells, Kupffer cells, neurons, astrocytes, oligodendroglia, enterocytes, Schwann cells, ganglion cells, renal tubular cells, pancreatic acinar, duct and islet cells, epidermal cells, splenic sinusoidal endothelial cells, lymphocytes, and fibroblasts.

### Methods

### Peptide Selection and Antibody Production

The HGPRBMY4 sequence was analyzed using the algorithm of Hopp and Woods (Proc. Nat. Acad. Sci. USA 78(6): 3824-3828 (1981)) in order to determine candidate peptides for use in antibody production. These peptides were compared to sequences within the Swissprot database in order to assess the likely specificity of the resulting antibodies. The following peptide was selected and synthesized and used to generate rabbit polyclonal antisera: KEIRQRILRLFHVATHASE (SEQ ID NO: 64). In order to allow for peptide conjugation to the carrier protein, a cysteine residue was added to the N-terminus of the peptide. The serum from the third bleed was subjected to peptide affinity purification, and the eluted antibodies were then used in immunohistochemistry experiments.

Antibody Titration Protocol and Results of Positive Control Study:

Titration experiments were conducted with antibody HGPRBMY4 (rabbit polyclonal) to determine concentrations that produce minimal background and maximal detection of signal. Serial dilutions were performed at 1:50, 1:100, 1:250, 1:500, and 1:1000. The highest signal-to-noise ratios were apparent at dilutions of 1:100 and 1:250 on paraffin-embedded, formalin-fixed tissues. These concentrations were used for the study. The antibody directed against HGPRBMY4 was used as the primary antibody, and the principal detection system consisted of a Vector anti-rabbit secondary (BA-1000), a Vector ABC-AP kit (AK-5000), and a Vector Red substrate kit (SK-5100). These reagents produced a fuchsia-colored deposit in areas of antibody binding. Tissues were also stained with a positive control antibody (CD31) to verify that the tissue antigens were preserved and accessible for immunohistochemical analysis. Only tissues that stained positive for CD31 were used for the remainder of this study. The negative control consisted of performing the entire immunohistochemistry procedure on adjacent sections in the absence of primary antibody. Slides were imaged using a DVC 1310C digital camera coupled to a Nikon microscope. Images were stored as TIFF files using Adobe Photoshop. staining a body standard panel I are as follows:

BODY STANDARD PANEL I

| | Sample | Tissue | Diagnosis | Age/Sex |
|---|---|---|---|---|
| 1 | 1 | Brain, | Normal | 53 M |
| | | Cortex | | |
| 2 | 1 | Heart | Normal | 81 F |
| 3 | 1 | Kidney, | Normal | 63 M |
| | | Cortex | | |
| 4 | 1 | Kidney, | Normal | 63 M |
| | | Medulla | | |
| 5 | 1 | Liver | Normal | 62 M |
| 6 | 1 | Lung | Normal | 15 M |
| 7 | 1 | Pancreas | Normal | 61 M |
| 8 | 1 | Skeletal | Normal | 56 M |
| | | Muscle | | |
| 9 | 1 | Skin | Normal | 18 F |
| 10 | 1 | Small | Normal | 66 F |
| | | Intestine | | |
| 11 | 1 | Spleen | Normal | 57 M |

Sample 1 was a section of normal cerebral cortex obtained at autopsy from a 53-year-old male who died of a ruptured aneurysm of the aortic arch. The H&E (hematoxylin and eosin stain) section showed cerebral cortex with unremarkable neurons and astroglia. Normal pia-arachnoid meninges were present with small blood vessel. In section stained with HGPRBMY4 antibody, neurons within the cortex were predominanantly negative, except for subsets that showed blush punctate nuclear sraining. Astrocytes were negative, except for subsets that showed blush punctate nuclear staining. Oligodendrocytes and capillary endothelial cells were negative. Within white matter, astrocytes were negative, except for subsets that showed blush punctate nuclear staining. Oligodendrocytes and microglial cells were negative. Within meninges, meningothelial cells and subpial astroglia were negative.

### Heart, Normal

Sample 1 was a section of normal heart obtained at autopsy from an 81-year-old female who died of complications of atherosclerotic cardiovascular disease. The H&E (hematoxylin and eosin stain) section showed unremarkable myocardium with small branches of the coronary artery and vein within the tissue. No endocardium or pericardium was present. In sections stained with HGPRBMY4 antibody , cardiac myocytes were predominantly negative, except for rare, blush, punctate granules intermixed with lipofucsin pigment in the cytoplasm. Capillary endothelium was predominantly negative, with only rare focal blush staining. Interstitial fibroblasts were negative. Within muscular vessels, endothelium and vascular smooth muscle were negative.

### Kidney, Cortex, Normal

Sample 1 was a section of normal renal cortex obtained at surgery from a 63-year-old male. The H&E (hematoxylin and eosin stain) sections showed normal renal cortex without inflammation or fibrosis. In sections stained with HGPRBMY4 antibody, within glomeruli, the epithelium lining Bowman's capsule was strongly positive, and visceral epithelial cells were negative or showed blush staining. The epithelium of proximal convoluted tubules was predominantly negative, with only rare blush to strong positivity. Distal convoluted tubules were mostly negative, but subsets showed blush to faint positivity, and collecting ducts were predominantly negative with rare focal blush positivity.

### Kidney, Medulla, Normal

Sample 1 was a section of normal renal medulla obtained at surgery from a 63-year-old male. The H&E (hematoxylin and eosin stain) section showed normal renal medulla with a mildly hyalinized interstitium. In sections stained with HGPRBMY4 antibody, within the renal medulla, collecting ducts were negative or stained faintly to strongly, and thin loops of Henle were negative. Thick loops of Henle were negative. Vascular endothelium was negative, and vascular smooth muscle stained faintly to moderately.

### Liver, Normal

Sample 1 was a section of normal liver obtained at autopsy from a 62-year-old male who died of a myocardial infarction. The H&E (hematoxylin and eosin stain) section showed normal liver with scattered chronic inflammatory cells in the portal region. In sections stained with HGPRBMY4 antibody, hepatocytes were predominantly negative, but occasional subsets showed blush to faint staining and rare cells showed moderate to strong staining. Sinusoidal endothelial cells and Kupffer cells were negative. Within portal areas, bile duct epithelium was negative. Within branches of the hepatic artery and portal vein, endothelial cells and vascular smooth muscle were negative.

### Lung, Normal

Sample 1 was a section of normal lung obtained at autopsy from a 15-year-old male who died of trauma associated with a motor-vehicle accident. The H&E (hematoxylin and eosin stain) section showed atelectatic lung and pleura with focal alveolar hemorrhage. Although alveolar septa and other parenchymal structures appeared normal with no inflammation (except for occasional macrophages), type II pneumocytes were highly represented, consistent with reactive changes against extravasated erythrocytes in the alveolar lumina. In sections stained with HGPRBMY4 antibody, type I pneumocytes were negative or showed blush staining, and type II pneumocytes showed blush to moderate staining. Alveolar capillary endothelium was negative. Alveolar macrophages showed blush to faint staining. Vascular endothelium was negative or showed blush to faint staining, and vascular smooth muscle stained faintly. Mesothelial cells stained moderately to strongly.

### Pancreas, Normal

Sample 1 was a section of normal pancreas obtained at autopsy from a 61-year-old male who died of coronary sclerosis with stenosis. The H&E (hematoxylin and eosin stain) section showed normal pancreas with duct, acinar, and islet tissue present. In sections stained with HGPRBMY4 antibody, pancreatic exocrine acinar epithelium and ducts were negative. Cells within the islets of Langerhans were negative or showed rare blush staining. Vascular endothelium was negative or showed blush to faint staining. Vascular smooth muscle and adipocytes were negative.

### Skeletal Muscle, Normal

Sample 1 was a section of normal skeletal muscle obtained at autopsy from a 56-year-old male who died of an intracranial hemorrhage. The H&E (hematoxylin and eosin stain) section consisted of normal skeletal muscle and endomysial fibrovascular tissue, but no perimysium was present. In sections stained with HGPRBMY4 antibody, skeletal muscle myocytes were negative or showed blush to moderate staining, occasionally along striations. Subsets of myocytes were completely negative adjacent to other myocytes, which were moderately positive (suspicious for possible differential staining of types I and II myocytes). Within the endomysium, capillary endothelium was negative. Fibroblasts were negative.

### Skin, Normal

Sample 1 was a section of normal skin obtained at breast excision from an 18-year-old female. The H&E (hematoxylin and eosin stain) section showed normal epidermis, and dermis with adnexal structures. In sections stained with HGPRBMY4 antibody, within the epidermis, basal keratinocytes, cells within the stratum spinosum, and cells within the stratum granulosum were negative. Corneal keratin, melanocytes, and Langerhans cells were negative. Sebocytes within sebaceous glands were faintly to moderately positive. Dermal fibroblasts were negative, and within dermal vessels, endothelium and vascular smooth muscle were negative. The arrector pili muscles were moderately to strongly positive. Scattered neutrophils were strongly positive.

### Small Intestine, Normal

Sample 1 was a section of normal small intestine obtained at surgery from a 66-year-old female. The H&E (hematoxylin and eosin stain) section of ileum showed normal-appearing epithelium and scattered chronic inflammatory cells in the lamina propria with moderate villous edema. Normal-appearing submucosa, muscularis mucosa, and muscularis propria were present. In sections stained with HGPRBMY4 antibody, enterocytes, neuroendocrine cells, and goblet cells were negative. Within the lamina propria, capillary endothelium was negative, the majority of plasma cells were negative or showed blush staining, and macrophages showed faint punctate positivity in their cytoplasm. The smooth muscle of the muscularis mucosa and muscularis propria showed predominantly blush to faint staining. Endothelial cells within submucosal vessels were negative, and vascular smooth muscle was negative. Neutrophils were strongly positive. Lymphocytes were negative. Within Auerbach's and Meissner's plexuses, ganglion cells and Schwann cells were negative. The majority of fibroblasts were negative.

### Spleen, Normal

Sample 1 was a section of normal spleen obtained at autopsy from a 57-year-old male who died of a cerebrovascular accident. The H&E (hematoxylin and eosin stain) section consisted of normal spleen with the red and white pulp, without diagnostic abnormality. In sections stained within HGPRBMY4 antibody, within the white pulp, lymphocytes in periarterial lymphatic sheaths were negative or showed blush (granular nuclear) staining. Within the red pulp, sinusoidal endothelial cells and reticular cells were negative. Eosinophils and neutrophils were strongly positive. Within vessels, endothelial cells and smooth muscle were negative. Plasma cells were nagative. Mesothelial cells on the capsular serosal surface were predominantly nagetive, with occasional subsets being strongly positive.

The result of staining individual specimens are as follows.

Individual Specimen Panel

| | Sample | Tissue | Diagnosis | Age/Sex |
|---|---|---|---|---|
| 1 | 1 | Prostate | Normal | 40 M |
| | 2 | Prostate | Normal | 13 M |
| | 3 | Prostate | Normal | 16 M |
| | 4 | Prostate | Normal | 65 M |
| | 5 | Prostate | Normal | 18 M |
| 2 | 1 | Prostate | Benign Prostatic Hyperplasia | 71 M |
| | 2 | Prostate | Benign Prostatic Hyperplasia | 77 M |
| | 3 | Prostate | Benign Prostatic Hyperplasia | 82 M |
| 3 | 1 | Prostate | Carcinoma | 77 M |
| | 2 | Prostate | Carcinoma | 58 M |
| | 3 | Prostate | Carcinoma | 72 M |
| | 4 | Prostate | Carcinoma | 61 M |
| | 5 | Prostate | Carcinoma | 72 M |

Sample 1 was a section of normal prostate obtained at autopsy from a 40-year-old male who died of acute interstitial pneumonitis. The H&E (hematoxylin and eosin stain) stained section showed normal prostatic glandular and stromal tissue with concretion occasionally present within dilated glandular lumina. In sections stained with HGPRBMY4 antibody, prostatic glandular epithelium was mostly negative, with occasional subset showing faint to moderate positivity. Ductal epithelium was predominantly nagative, with only rare faintly positive ducts. Basal cells were negative. Prostatic stromal smooth muscle myocytes were predominantly negative or showed rare strong positivity. Stromal fibroblasts were negative or showed rare strong positivity. Vascular endothelial cells and vascular smooth muscle were negative. Concretions were negative or showed rare blush staining.

Sample 2 was a section of normal prostate obtained at autopsy from a 13-year-old male who died of pulmonary hemorrhage secondary to malignant lymphoma. The H&E (hematoxylin and eosin stain) section showed normal prostatic glandular and stromal tissue as well as adjacent fibrovascular and peripheral nerve tissue. In sections stained with HGPRBMY4 antibody, prostatic glandular epithelium, ductal epithelium, and basal cells stained faintly. Prostatic stromal smooth muscle myocytes were mostly negative, with only rare focal blush to faint staining. Stromal fibroblasts were negative. Vascular endothelial cells were negative, and vascular smooth muscle was negative or showed rare blush to faint positivity. Schwann cells and adipocytes were negative.

Sample 3 was a section of normal prostate obtained at autopsy from a 16-year-old male who died of trauma. The H&E (hematoxylin and eosin stain) stained section showed normal prostatic glandular and stromal tissue, as well as prostatic capsule and adjacent fibrovascular, peripheral nerve, and ganglion tissue. In sections stained with HGPRBMY4 antibody, prostatic glandular epithelium was faintly positive, and ductal epithelium was mostly negative, with subsets of cells showing faint staining. Basal cells were negative or showed blush staining. Prostatic stromal smooth muscle myocytes, stromal fibroblasts, vascular endothelial and vascular smooth muscle, prostatic capsule fibroblasts, Schwann cells, ganglion cells, and adipocytes were negative.

Sample 4 was a section of normal prostate obtained at surgery from a 65-year-old male. The H&E (hematoxylin and eosin stain) sections showed benign prostatic glandular and stromal tissue with a focal suggestion of early nodule formation, but non-diagnostic of hyperplasia. Adjacent normal fibrovascular and peripheral nerve tissue was also present. In sections stained with HGPRBMY4 antibody, prostatic glandular epithelium was faintly positive, with rare glands showing moderate staining. Most of the staining was limited to the luminal ends of the epithelial cells. Ductal epithelium was faintly positive. Basal cells were predominantly negative with subsets showing blush to faint staining. Prostatic stromal smooth muscle myocytes were negative or showed blush to faint staining. Stromal fibroblasts were negative. Vascular endothelial cells were negative or showed blush staining, and vascular smooth muscle was faintly to moderately positive. The prostatic capsule fibroblasts were negative. Schwann cells were negative or showed blush staining, and adipocytes were negative.

Sample 5 was a section of normal prostate obtained at autopsy from an 18-year-old male who died of a gunshot wound. The H&E (hematoxylin and eosin stain) section showed normal prostatic glandular and stromal tissue, prostatic urethral urothelium, as well as adjacent fibrovascular, adipose, skeletal muscle, and peripheral nerve tissue. In sections stained with HGPRBMY4 antibody, prostatic glandular epithelium stained predominantly faintly, with subsets of glands showing moderate positivity. Staining was mostly limited to the luminal ends of epithelial cells. Ductal epithelium stained faintly, in contrast to adjacent prostatic urethral urothelium, which was predominantly negative. Basal cells were predominantly negative, with subsets showing blush to faint staining. Prostatic stromal smooth muscle myocytes and stromal fibroblasts were negative. Vascular endothelial cells were negative, and vascular smooth muscle was negative or stained faintly in rare vessels within the extraprostatic tissue. Prostatic capsule fibroblasts were negative. Schwann cells were negative, and adipocytes were predominantly negative, with rare strongly positive subsets. Skeletal muscle myocytes were negative or showed faint to moderate staining.

### Prostate, Benign Prostatic Hyperplasia

Sample 1 was a section of prostate obtained at surgery from a 71-year-old male with benign prostatic hyperplasia. The H&E (hematoxylin and eosin stain) section showed fragments of benign prostatic tissue with focal glandular and stromal nodular hyperplasia. Focal chronic inflammation with lymphoid follicle formation and intraductal acute fibrinopurulent exudate was also present. Low-grade PIN (Prostatic Intraepithelial Neoplasia) was noted focally in one nodule. In sections stained with HGPRBMY4 antibody, prostatic glandular epithelium showed predominantly faint staining, with subsets of moderately positive glands. No difference between areas of PIN and non-dysplastic regions was identified. Most staining was near luminal ends of epithelial cells. Ductal epithelium was faintly positive, and the adjacent prostatic urethral urothelium was faintly to strongly positive (associated with mixed inflammation having strongly positive neutrophils, and lymphocytes that were negative to blush positive). Interestingly, basal cells showed predominantly moderate staining, with subsets showing blush to faint staining. Myocytes of prostatic stromal smooth muscle were negative or showed blush to faint staining. Stromal fibroblasts were negative or showed blush staining. Vascular endothelial cells were negative or showed blush to moderate positivity, and vascular smooth muscle stained faintly.

Sample 2 was a section of prostate obtained at surgery from a 77-year-old male with benign prostatic hyperplasia. The H&E (hematoxylin and eosin stain) section showed stromal and glandular nodular hyperplasia, cystic dilatation of glands, and scattered chronic inflammation. No PIN was identified. In sections stained with HGPRBMY4 antibody, prostatic glandular epithelium showed predominantly faint staining with subsets of glands showing moderate positivity. Staining was mostly limited to the luminal ends of some of the epithelial cells, and other staining was evenly distributed throughout the cytoplasm. Interestingly, in areas of hyperplasia, basal cells showed predominantly moderate positivity, with subsets showing blush to faint staining. Prostatic stromal smooth muscle myocytes were negative or showed blush to faint staining. Stromal fibroblasts were negative or showed blush staining. Vascular endothelial cells were negative or showed blush to moderate positivity, and vascular smooth muscle stained faintly.

Sample 3 was a section of prostate obtained at surgery from an 82-year-old male with benign prostatic hyperplasia. The H&E (hematoxylin and eosin stain) section showed glandular and stromal nodular hyperplasia with focal low-grade PIN. Focal cystically dilated glands were present with scattered chronic inflammation in the stroma. In sections stained with HGPRBMY4 antibody, prostatic glandular epithelium showed predominantly faint staining, with subsets of moderately positive glands. Staining was mostly limited to the luminal ends of some of the epithelial cells, and other staining was evenly distributed throughout the cytoplasm. Interestingly, in some areas of hyperplasia, basal cells were predominantly moderately positive with subsets showing blush to faint staining. Other nodules showed about the same intensity of staining in both basal and epithelial cells. No difference in staining was noted between PIN cells and non-dysplastic cells. Prostatic stromal smooth muscle myocytes were negative or showed blush to faint staining. Stromal fibroblasts were negative or showed blush staining. Vascular endothelial cells were negative or showed blush staining, and vascular smooth muscle showed blush to faint staining.

### Prostate, Carcinoma

Sample 1 was a section of prostate obtained at surgery from a 77-year-old male with prostate carcinoma. The H&E (hematoxylin and eosin stain) section showed atypical glands infiltrating the fibromuscular stroma, as well as foci of fused glands with minimal lumen formation. These findings were diagnostic of moderately to poorly differentiated adenocarcinoma (Gleason grade 3+4=7). In sections stained with HGPRBMY4 antibody, malignant cells in moderately differentiated (Gleason pattern 3) glands showed predominantly faint to strong staining, with only a small negative subset. Moderately to poorly differentiated glands (Gleason pattern 4), however, were predominantly negative, with only a small subset containing mainly blush, but rarely strongly positive cells. Smooth muscle stroma myocytes showed blush to faint staining in the region of tumor. Stromal fibroblasts were negative in the region of tumor. Vascular endothelial cells showed blush to strong staining, and vascular smooth muscle was faintly to moderately positive.

Sample 2 was a section of prostate obtained at surgery from a 58-year-old male with prostate carcinoma. The H&E (hematoxylin and eosin stain) section showed infiltrating glands of varying size, nuclear and architectural atypia diagnostic of moderately differentiated adenocarcinoma (Gleason grade 3+3=6). In sections stained with HGPRBMY4 antibody, malignant cells stained faintly to strongly, although a small subset of glands were negative or showed blush staining. Smooth muscle stromal myocytes stained faintly to moderately near the tumor, compared to predominantly negative with only occasional blush to faint (rarely moderate) staining, distant from the tumor. Fibroblasts were negative. Vascular endothelial cells were negative or showed blush staining, and vascular smooth muscle showed blush to faint staining.

Sample 3 was a section of prostate obtained at surgery from a 72-year-old male with prostate carcinoma. The H&E (hematoxylin and eosin stain) stained section showed infiltrating atypical glands of variable size and shape diagnostic of moderately differentiated adenocarcinoma (Gleason grade 3+3=6). Surrounding prostate glands focally contained high-grade PIN. In sections stained with HGPRBMY4 antibody, malignant cells stained faintly to strongly, although a small subset of glands were negative or showed blush staining. High-grade PIN in the surrounding prostate glands stained faintly to moderately positive in epithelial cells and basal cells. Smooth muscle stromal myocytes stained faintly to moderately, independent of their proximity to tumor. Fibroblasts were negative. Vascular endothelial cells were negative or showed blush staining, and vascular smooth muscle showed blush to faint staining.

Sample 4 was a section of prostate obtained at surgery from a 61-year-old male with prostate carcinoma. The H&E (hematoxylin and cosin stain) section showed infiltrating atypical glands with nuclear and architectural atypia. A subset of glands had minimal or no lumina with focal perineural invasion. These findings were diagnostic of moderately to poorly differentiated adenocarcinoma (Gleason grade 3+4=7). In sections stained with HGPRBMY4 antibody, malignant cells were negative or stained faintly to moderately positive. Generally, more differentiated glands (Gleason pattern 3) showed blush to moderate staining, whereas less differentiated glands (Gleason pattern 4), were more often negative or stained blush. However, rare cell clusters stained moderately positive. Smooth muscle stromal myocytes stained faintly to moderately positive, independent of their proximity to the tumor. Fibroblasts were negative. Vascular endothelial cells were negative or showed blush staining, and vascular smooth muscle showed blush to moderate staining.

Sample 5 was a section of prostate obtained at surgery from a 72-year-old male with prostate carcinoma. The H&E (hematoxylin and eosin stain) section showed infiltrating atypical glands with variable size and shape, but with retention of the glandular lumina and architecture. Focal perineural invasion was present. These findings were diagnostic of moderately differentiated adenocarcinoma (Gleason grade 3+3=6). In sections stained with HGPRBMY4 antibody, malignant cells were negative or stained faintly to moderately positive. Rare malignant cells stained strongly. Smooth muscle stromal myocytes were negative or showed blush to moderate staining, independent of their proximity to the tumor. Fibroblasts were negative. Vascular endothelial cells were negative or showed blush to moderate staining, and vascular smooth muscle stained faintly to moderately.

### Example 5

### G-protien Coupled Receptor PCR Expression Profiling

RNA quantification was performed using the Taqman® real-time-PCR fluorogenic assay. The Taqman® assay is one of the most precise methods for assaying the concentration of nucleic acid templates.

All cell lines were grown using standard conditions: RPMI 1640 supplemented with 10% fetal bovine serum, 100 IU/ml penicillin, 100 mg/ml streptomycin, and 2 mM L-glutamine, 10 mM Hepes (all from GibcoBRL; Rockville, Md.). Eighty percent confluent cells were washed twice with phosphate-buffered saline (GibcoBRL) and harvested using 0.25% trypsin (GibcoBRL). RNA was prepared using the RNeasy Maxi Kit from Qiagen (Valencia, Calif.).

cDNA template for real-time PCR was generated using the Superscript™ First Strand Synthesis system for RT-PCR.

SYBR Green real-time PCR reactions were prepared as follows: The reaction mix consisted of 20 ng first strand cDNA; 50 nM Forward Primer; 50 nM Reverse Primer; 0.75×SYBR Green I (Sigma); 1×SYBR Green PCR Buffer (50 mM Tris-HCl pH 8.3, 75 mM KCl); 10% DMSO; 3 mM MgCl₂; 300 micromolar each dATP, dGTP, dTTP, dCTP; 1 U Platinum® Taq DNA Polymerase High Fidelity (Cat# 11304-029; Life Technologies; Rockville, Md.); 1:50 dilution; ROX (Life Technologies). Real-time PCR was performed using an Applied Biosystems 5700 Sequence Detection System. Conditions were 95° C. for 10 min (denaturation and activation of Platinum® Taq DNA Polymerase), 40 cycles of PCR (95° C. for 15 sec, 60° C. for 1 min). PCR products are analyzed for uniform melting using an analysis algorithm built into the 5700 Sequence Detection System.

Forward primer: GPCR9-F1: 5′-CCTGTGCTCAACCCAATTGTCT-3′ (SEQ ID NO: 25); and

Reverse primer: GPCR9-R: 15′-ACTGACACCTAGGGCTCTGAAG-3′ (SEQ ID NO: 26).

cDNA quantification used in the normalization of template quantity was performed using Taqman® technology. Taqman® reactions are prepared as follows: The reaction mix consisted of 20 ng first strand cDNA; 25 nM GAPDH-F3, Forward Primer; 250 nM GAPDH-R1 Reverse Primer; 200 nM GAPDH-PVIC Taqman® Probe (fluorescent dye labeled oligonucleotide primer); 1×Buffer A (Applied Biosystems); 5.5 mM MgCl2; 300 micromolar dATP, dGTP, dTTP, dCTP; 1 U Amplitaq Gold (Applied Biosystems). GAPDH, D-glyceraldehyde-3-phosphate dehydrogenase, was used as control to normalize mRNA levels.

Real-time PCR was performed using an Applied Biosystems 7700 Sequence Detection System. Conditions were 95° C. for 10 min. (denaturation and activation of Amplitaq Gold), 40 cycles of PCR (95° C. for 15 sec, 60° C. for 1 min).

The sequences for the GAPDH oligonucleotides used in the Taqman® reactions are as follows:

| | |
|---|---|
| GAPDH-F3-5′-AGCCGAGCCACATCGCT-3′ | (SEQ ID NO: 27) |
| GAPDH-R1-5′-GTGACCAGGCGCCCAATAC-3′ | (SEQ ID NO: 28) |
| GAPDH-PVIC Taqman ® Probe-VIC-5′- | (SEQ ID NO: 29) |
| CAAATCCGTTGACTCCGACCTTCACCTT-3′ | |
| TAMRA. | |

The Sequence Detection System generates a Ct (threshold cycle) value that is used to calculate a concentration for each input cDNA template. cDNA levels for each gene of interest are normalized to GAPDH cDNA levels to compensate for variations in total cDNA quantity in the input sample. This is done by generating GAPDH Ct values for each cell line. Ct values for the gene of interest and GAPDH are inserted into a modified version of the δδCt equation (Applied Biosystems Prism® 7700 Sequence Detection System User Bulletin #2), which is used to calculate a GAPDH normalized relative cDNA level for each specific cDNA. The δδCt equation is as follows: relative quantity of nucleic acid template=2^{δδCt}=2^{(δCta-δCtb)}, where δCta=Ct target−Ct GAPDH, and δCtb=Ct reference−Ct GAPDH. (No reference cell line was used for the calculation of relative quantity; δCtb was defined as 21).

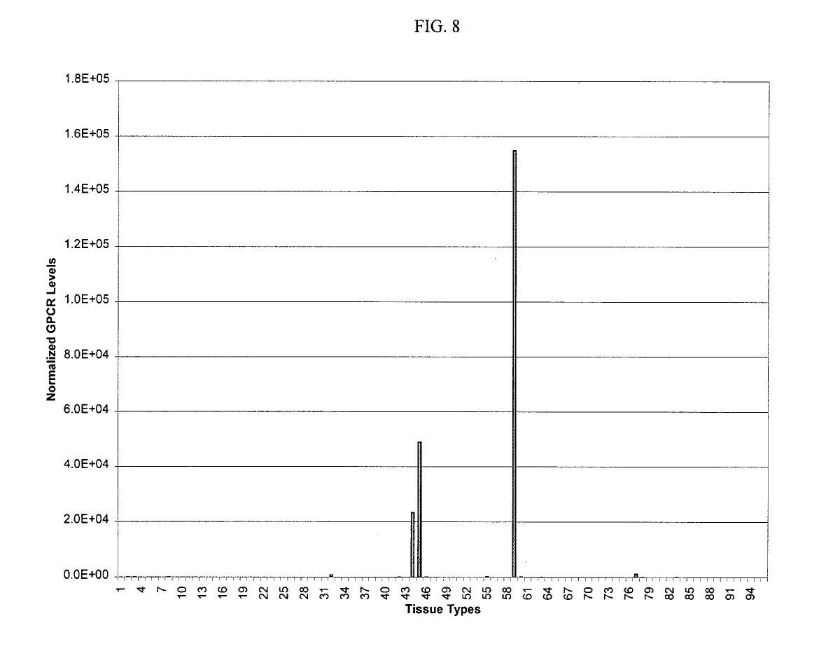

The Graph # of Table I corresponds to the tissue type position number of FIG. 8. Interestingly, HGPRBMY4 (also known as GPCR9) was found to be overexpressed 800 to 49,000 fold greater in colon carcinoma cell lines and 150,000 in the SHIP-77 lung carcinoma cell line, in comparison to other cancer cell lines in the OCLP-1 (oncology cell line panel).

TABLE I

| Graph # | Name | Tissue | CtGAPDH | GPCR9-1 | dCt | ddCt | Quant. |
|---|---|---|---|---|---|---|---|
| 1 | A-427 | lung | 18 | 40 | 22 | 1 | 5.0E−01 |
| 2 | A431 | squamous | 19.85 | 36.19 | 16.34 | −4.66 | 2.5E+01 |
| 3 | A2780/DDP-S | ovarian | 17.89 | 33.7 | 15.81 | −5.19 | 3.7E+01 |
| 4 | A2780/DDP-R | ovarian | 21.51 | 40 | 18.49 | −2.51 | 5.7E+00 |
| 5 | HCT116/epo5 | colon | 17.71 | 40 | 22.29 | 1.29 | 4.1E−01 |
| 6 | A2780/TAX-R | ovarian | 18.4 | 37.62 | 19.22 | −1.78 | 3.4E+00 |
| 7 | A2780/TAX-S | ovarian | 17.83 | 40 | 22.17 | 1.17 | 4.4E−01 |
| 8 | A549 | lung | 17.63 | 32.77 | 15.14 | −5.86 | 5.8E+01 |
| 9 | AIN4/myc | breast | 17.81 | 40 | 22.19 | 1.19 | 4.4E−01 |
| 10 | AIN 4T | breast | 17.15 | 37.06 | 19.91 | −1.09 | 2.1E+00 |
| 11 | AIN 4 | breast | 17.49 | 40 | 22.51 | 1.51 | 3.5E−01 |
| 12 | BT-549 | breast | 17.55 | 40 | 22.45 | 1.45 | 3.7E−01 |
| 13 | BT-20 | breast | 17.9 | 40 | 22.1 | 1.1 | 4.7E−01 |
| 14 | C-33A | cervical | 17.49 | 40 | 22.51 | 1.51 | 3.5E−01 |
| 15 | CACO-2 | colon | 17.56 | 37.61 | 20.05 | −0.95 | 1.9E+00 |
| 16 | Calu-3 | lung | 18.09 | 40 | 21.91 | 0.91 | 5.3E−01 |
| 17 | Calu-6 | lung | 16.62 | 40 | 23.38 | 2.38 | 1.9E−01 |
| 18 | BT-474 | breast | 17.65 | 35.54 | 17.89 | −3.11 | 8.6E+00 |
| 19 | Cx-1 | colon | 18.79 | 40 | 21.21 | 0.21 | 8.6E−01 |
| 20 | CCRF-CEM | leukemia | 17.07 | 38.51 | 21.44 | 0.44 | 7.4E−01 |
| 21 | ChaGo-K-1 | lung | 17.79 | 40 | 22.21 | 1.21 | 4.3E−01 |
| 22 | DU4475 | breast | 18.1 | 40 | 21.9 | 0.9 | 5.4E−01 |
| 23 | ES-2 | ovarian | 17.22 | 36.83 | 19.61 | −1.39 | 2.6E+00 |
| 24 | H3396 | breast | 18.04 | 40 | 21.96 | 0.96 | 5.1E−01 |
| 25 | HBL100 | breast | 17.02 | 34.52 | 17.5 | −3.5 | 1.1E+01 |
| 26 | HCT116/VM46 | colon | 17.87 | 35.35 | 17.48 | −3.52 | 1.1E+01 |
| 27 | HCT116/VP35 | colon | 17.3 | 40 | 22.7 | 1.7 | 3.1E−01 |
| 28 | HCT116 | colon | 17.59 | 35.57 | 17.98 | −3.02 | 8.1E+00 |
| 29 | A2780/epo5 | ovarian | 17.54 | 34.65 | 17.11 | −3.89 | 1.5E+01 |
| 30 | HCT116/ras | colon | 17.18 | 40 | 22.82 | 1.82 | 2.8E−01 |
| 31 | HCT116/TX15 | colon | 17.36 | 36.41 | 19.05 | −1.95 | 3.9E+00 |
| | CR | | | | | | |
| 32 | HT-29 | colon | 17.9 | 29.26 | 11.36 | −9.64 | 8.0E+02 |
| 33 | HeLa | cervical | 17.59 | 35.15 | 17.56 | −3.44 | 1.1E+01 |
| 34 | Her2 MCF-7 | breast | 19.26 | 40 | 20.74 | −0.26 | 1.2E+00 |
| 35 | HL-60 | leukemia | 17.54 | 35.64 | 18.1 | −2.9 | 7.5E+00 |
| 36 | HOC-76 | ovarian | 34.3 | 40 | 5.7 | −15.3 | Mouse |
| 37 | Hs 294T | melanoma | 17.73 | 40 | 22.27 | 1.27 | 4.1E−01 |
| 38 | HS 578T | breast | 17.83 | 34.93 | 17.1 | −3.9 | 1.5E+01 |
| 39 | HT-1080 | fibrosarcoma | 17.16 | 36.92 | 19.76 | −1.24 | 2.4E+00 |
| 40 | HCT116/vivo | colon | 17.7 | 34.61 | 16.91 | −4.09 | 1.7E+01 |
| 41 | HT-3 | cervical | 17.42 | 40 | 22.58 | 1.58 | 3.3E−01 |
| 42 | K562 | leukemia | 18.42 | 34.32 | 15.9 | −5.1 | 3.4E+01 |
| 43 | SiHa | cervical | 18.07 | 40 | 21.93 | 0.93 | 5.2E−01 |
| 44 | LNCAP | prostate | 18.17 | 24.67 | 6.5 | −14.5 | 2.3E+04 |
| 45 | LS 174T | colon | 17.93 | 23.35 | 5.42 | −15.58 | 4.9E+04 |
| 46 | LX-1 | lung | 18.17 | 34.32 | 16.15 | −4.85 | 2.9E+01 |
| 47 | MCF7 | breast | 17.83 | 40 | 22.17 | 1.17 | 4.4E−01 |
| 48 | MCF-7/AdrR | breast | 17.23 | 40 | 22.77 | 1.77 | 2.9E−01 |
| 49 | MDA-MB-175- | breast | 15.72 | 40 | 24.28 | 3.28 | 1.0E−01 |
| | VII | | | | | | |
| 50 | MDA-MB-231 | breast | 17.62 | 40 | 22.38 | 1.38 | 3.8E−01 |
| 51 | MDA-MB-453 | breast | 17.9 | 37.1 | 19.2 | −1.8 | 3.5E+00 |
| 52 | MDA-MB-468 | breast | 17.49 | 40 | 22.51 | 1.51 | 3.5E−01 |
| 53 | MDAH 2774 | breast | 16.87 | 35.7 | 18.83 | −2.17 | 4.5E+00 |
| 54 | ME-180 | cervical | 16.86 | 40 | 23.14 | 2.14 | 2.3E−01 |
| 55 | MIP | colon | 16.92 | 30.42 | 13.5 | −7.5 | 1.8E+02 |
| 56 | ddH2O | colon | 40 | 36.21 | −3.79 | −24.79 | ND |
| 57 | SK-CO-1 | colon | 17.75 | 40 | 22.25 | 1.25 | 4.2E−01 |
| 58 | LoVo | colon | 17.64 | 36.89 | 19.25 | −1.75 | 3.4E+00 |
| 59 | SHP-77 | lung | 18.66 | 22.42 | 3.76 | −17.24 | 1.5E+05 |
| 60 | T84 | colon | 16.44 | 29.81 | 13.37 | −7.63 | 2.0E+02 |
| 61 | BT-483 | breast | 17.45 | 40 | 22.55 | 1.55 | 3.4E−01 |
| 62 | CCD-18Co | colon, | 17.19 | 34.51 | 17.32 | −3.68 | 1.3E+01 |
| | | fibroblast | | | | | |
| 63 | Colo 320DM | colon | 17.01 | 32.24 | 15.23 | −5.77 | 5.5E+01 |
| 64 | DMS 114 | lung | 18.14 | 36.92 | 18.78 | −2.22 | 4.7E+00 |
| 65 | Sk-LU-1 | lung | 15.81 | 32.95 | 17.14 | −3.86 | 1.5E+01 |
| 66 | SK-MES-1 | lung | 17.1 | 40 | 22.9 | 1.9 | 2.7E−01 |
| 67 | SW1573 | lung | 17.14 | 37.94 | 20.8 | −0.2 | 1.1E+00 |
| 68 | SW 626 | ovarian | 16.94 | 40 | 23.06 | 2.06 | 2.4E−01 |
| 69 | SW1271 | lung | 16.45 | 40 | 23.55 | 2.55 | 1.7E−01 |
| 70 | SW756 | cervical | 15.59 | 40 | 24.41 | 3.41 | 9.4E−02 |
| 71 | SW900 | lung | 18.17 | 40 | 21.83 | 0.83 | 5.6E−01 |
| 72 | T47D | breast | 18.86 | 40 | 21.14 | 0.14 | 9.1E−01 |
| 73 | UACC-812 | breast | 17.06 | 40 | 22.94 | 1.94 | 2.6E−01 |
| 74 | UPN251 | ovarian | 17.69 | 40 | 22.31 | 1.31 | 4.0E−01 |
| 75 | ZR-75-1 | breast | 15.95 | 40 | 24.05 | 3.05 | 1.2E−01 |
| 76 | SKBR3 | breast | 17.12 | 40 | 22.88 | 1.88 | 2.7E−01 |
| 77 | SW403 | colon | 18.39 | 29.19 | 10.8 | −10.2 | 1.2E+03 |
| 78 | SW837 | colon | 18.35 | 34.65 | 16.3 | −4.7 | 2.6E+01 |
| 79 | CCD-112Co | colon | 18.03 | 34.95 | 16.92 | −4.08 | 1.7E+01 |
| 80 | Colo201 | colon | 17.89 | 40 | 22.11 | 1.11 | 4.6E−01 |
| 81 | PC-3 | prostate | 17.25 | 40 | 22.75 | 1.75 | 3.0E−01 |
| 82 | OVCAR-3 | ovarian | 17.09 | 40 | 22.91 | 1.91 | 2.7E−01 |
| 83 | SW480 | colon | 17 | 32.1 | 15.1 | −5.9 | 6.0E+01 |
| 84 | SW620 | colon | 17.16 | 34.74 | 17.58 | −3.42 | 1.1E+01 |
| 85 | SW1417 | colon | 17.22 | 40 | 22.78 | 1.78 | 2.9E−01 |
| 86 | Colo 205 | colon | 18.02 | 40 | 21.98 | 0.98 | 5.1E−01 |
| 87 | HCT-8 | colon | 17.44 | 35.76 | 18.32 | −2.68 | 6.4E+00 |
| 88 | PA-1 | ovarian | 17.33 | 40 | 22.67 | 1.67 | 3.1E−01 |
| 89 | CCD-33Co | colon | 17.07 | 35.25 | 18.18 | −2.82 | 7.1E+00 |
| 90 | MRC-5 | lung | 17.3 | 40 | 22.7 | 1.7 | 3.1E−01 |
| 91 | Pat-21 R60 | breast | 35.59 | 40 | 4.41 | −16.59 | ND |
| 92 | NCI-H596 | lung | 17.73 | 37.25 | 19.52 | −1.48 | 2.8E+00 |
| 93 | MSTO-211H | lung | 16.81 | 36.57 | 19.76 | −1.24 | 2.4E+00 |
| 94 | Caov-3 | ovarian | 15.5 | 40 | 24.5 | 3.5 | 8.8E−02 |
| 95 | Ca Ski | cervical | 17.38 | 40 | 22.62 | 1.62 | 3.3E−01 |
| 96 | LS123 | colon | 17.65 | 34.51 | 16.86 | −4.14 | 1.8E+01 |

### Example 6

### Taqman™ Quantitative PCR Analysis of HGPRBMY4

SYBR green quantitative PCR analysis of HGPRBMY4 demonstrated that this GPCR was expressed mainly in the prostate, heart and testis. Analysis of HGPRBMY4 by TaqMan™ quantitative PCR on an extended panel of tissue RNAs confirms and extends these observations.

The sequences for the HGPRBMY4 primer/probe set are as follows:

| | | |
|---|---|---|
| Forward Primer: | 5′-CATTGACTGCTCTTTGCTCATCA-3′ | (SEQ ID NO: 61) |
| Reverse Primer: | 5′-AATAACCGGTGTCAAGCATAAGC-3′ | (SEQ ID NO: 62) |
| Probe: | 5′-TGAATCCCCCAGCAAAGTGCCTAGAACATAATA-3′. | (SEQ ID NO: 63) |

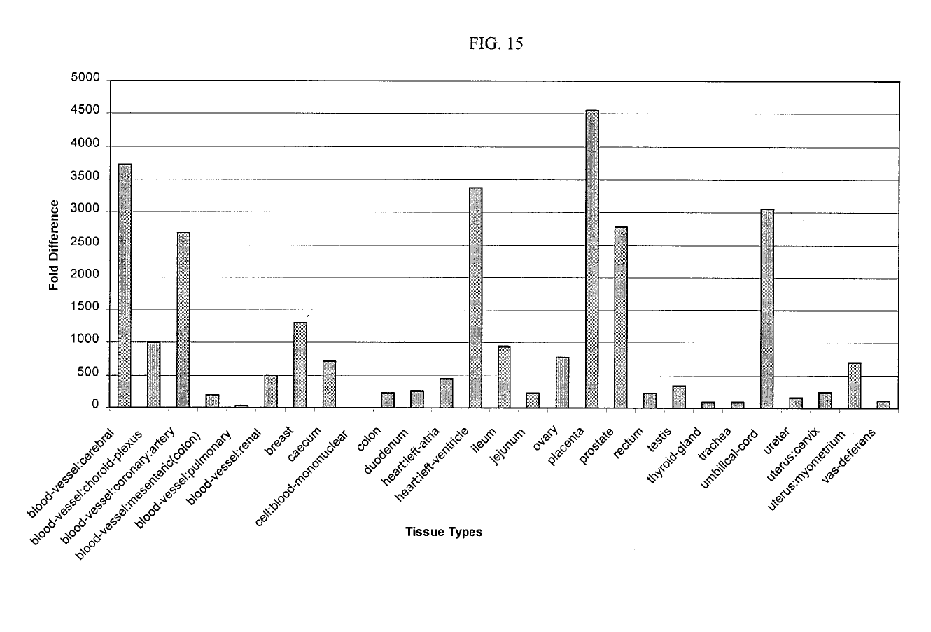

Transcripts of HGPRBMY4 are indeed found in the prostate, but higher concentrations are also observed in the placenta, cerebral blood vessel, and the umbilical cord. Within the heart, HGPRBMY4 is expressed approximately 7 times higher in the left ventricle when compared to the left atria. Analysis of HGPRBMY4 expression in RNA samples isolated from the left ventricle of patients with cardiomyopathy and hypertension found no evidence of altered expression in these conditions. Expression in the coronary artery is also appreciable however an analysis of HGPRBMY4 expression in samples isolated from individuals with atherosclerosis and hypertension again found no evidence of altered expression in these conditions (see FIG. 15).

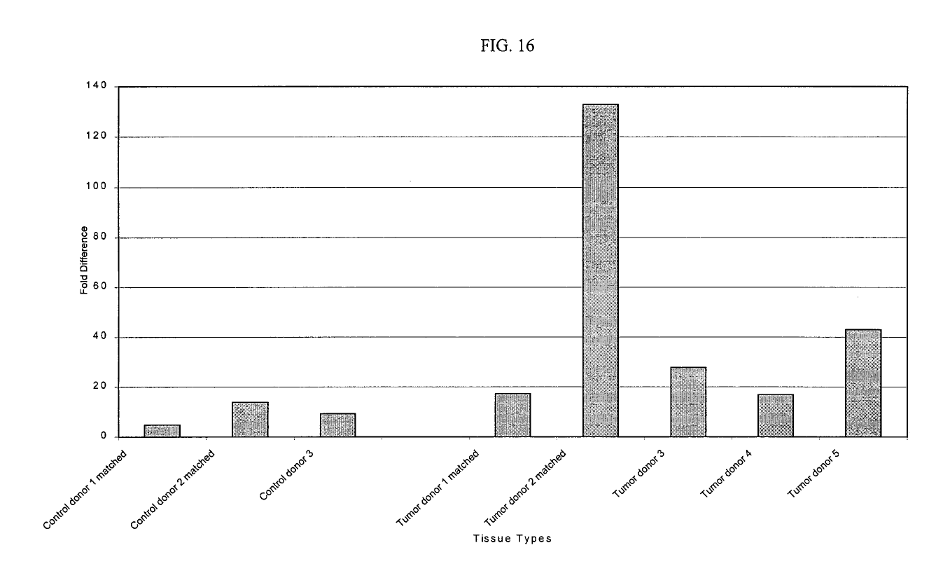

HGPRBMY4 expression has also been examined in RNA samples derived from normal and prostate tumors. In all tumors, expression of HGPRBMY4 was higher, including 2 matched samples where the increase was 3-fold in one sample and 10-fold in another. No other tumor type showed any evidence of altered expression. These data suggest that small molecule modulators of HGPRBMY4 can have utility in the treatment of prostate cancer (FIG. 16).

### Example 7

### SYBR Green Quantitative PCR Analysis of HGPRBMY4 in a Panel of Tumor Cell Lines

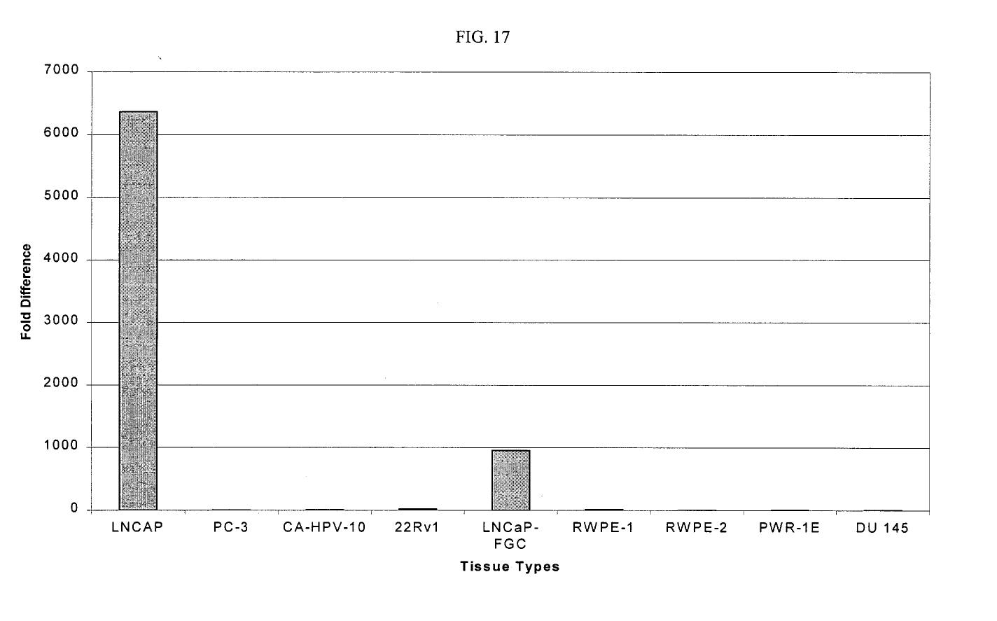

TaqMan™ quantitative PCR analysis of HGPRBMY4 has revealed that the transcript is expressed mainly in the prostate, heart, testis, placenta, cerebral blood vessel and umbilical cord. It was previously also shown that expression of HGPRBMY4 is higher in prostate tumor samples than in normal prostate samples. This analysis of several tumor cell lines confirms and extends these findings. HGPRBMY4 steady state RNA levels are over 6000 fold higher in the LNCAP prostate tumor cell line, and almost 1000 fold higher in the LNCAP-FGC prostate tumor cell line than the cell line with the lowest steady state levels. These findings support the suggestion that modulators of HGPRBMY4 can have utility in the treatment of prostate cancer (FIG. 17).

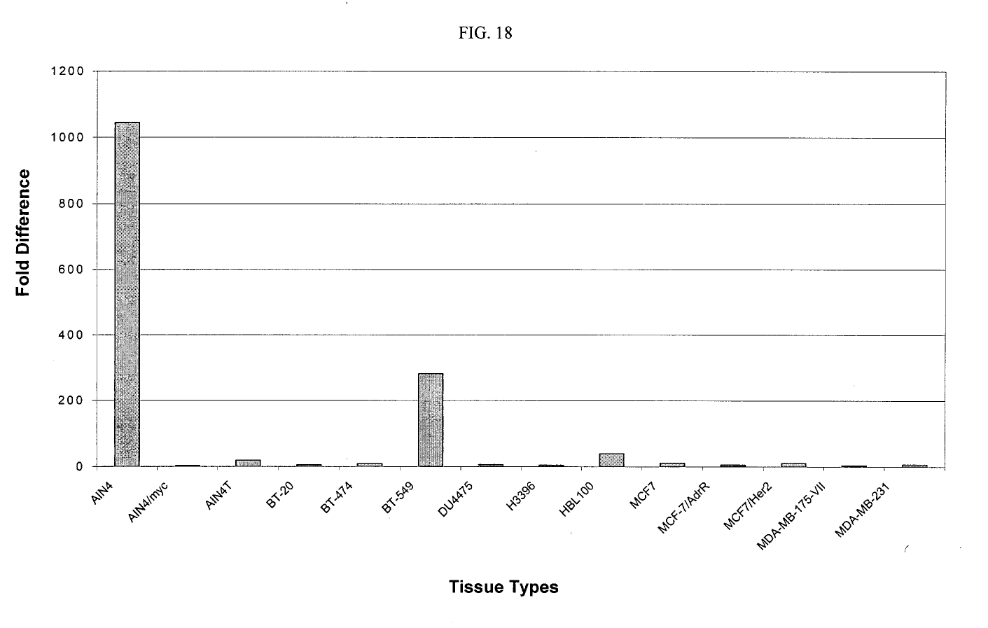

In addition to these findings, results also showed high steady state levels of HGPRBMY4 in cell lines derived from breast, colon and lung tumors HGPRBMY4 steady state RNA levels were over 1000 fold higher in the AIN4 line and almost 300 fold higher in the BT-549 line which are of breast origin (FIG. 18).

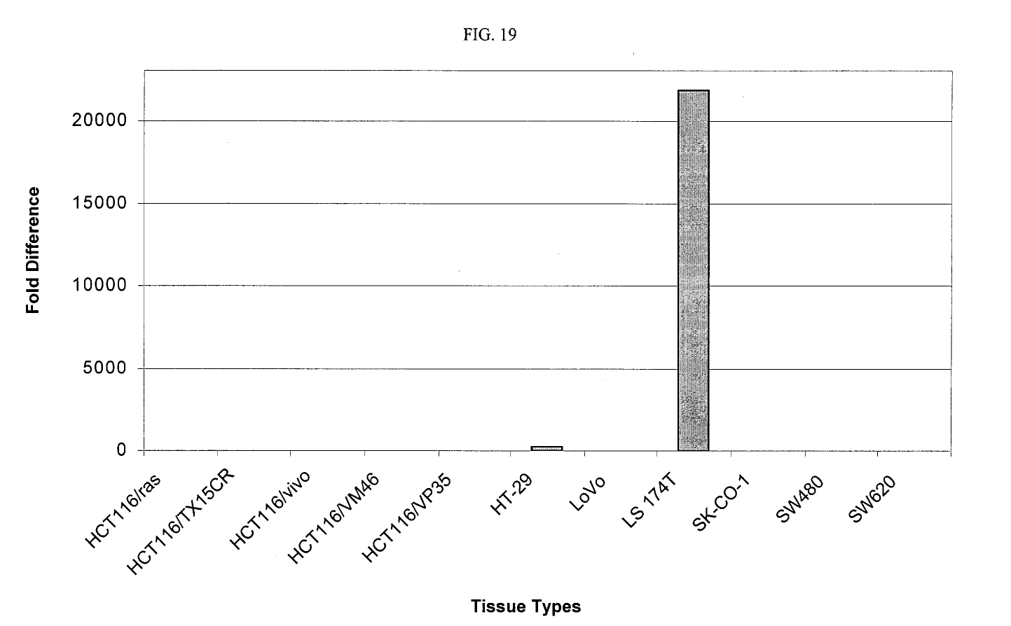

Steady state RNA levels forHGPRBMY4 were almost 22,000 fold higher in the LS174T cell line and over 250 fold higher in the HT-29 cell line which are of colon origin (FIG. 19).

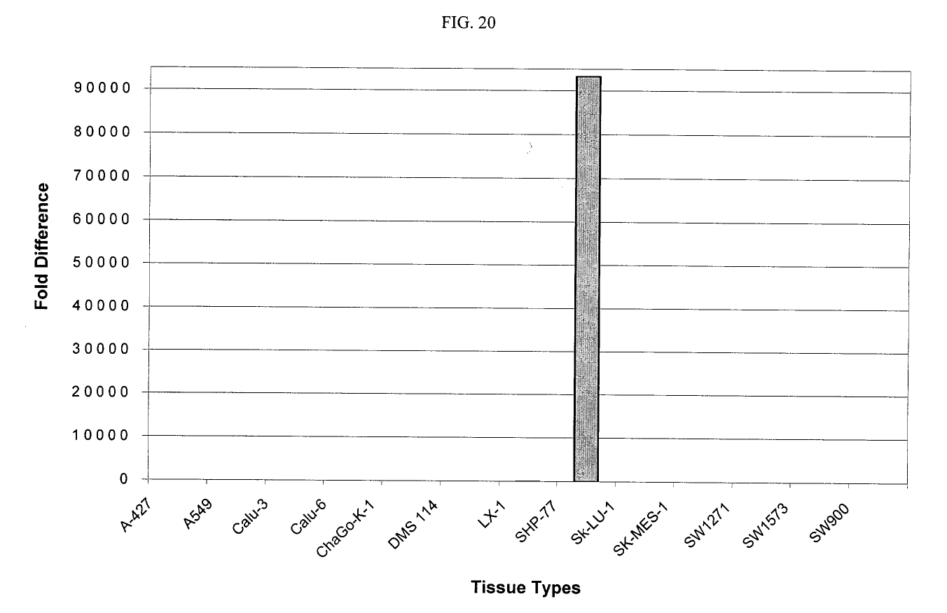

Steady state RNA levels of HGPRBMY4 were over 93,000 old higher in SHP-77 cell line, which is of lung origin, than that observed in the cell line with the lowest steady state RNA levels (FIG. 20).

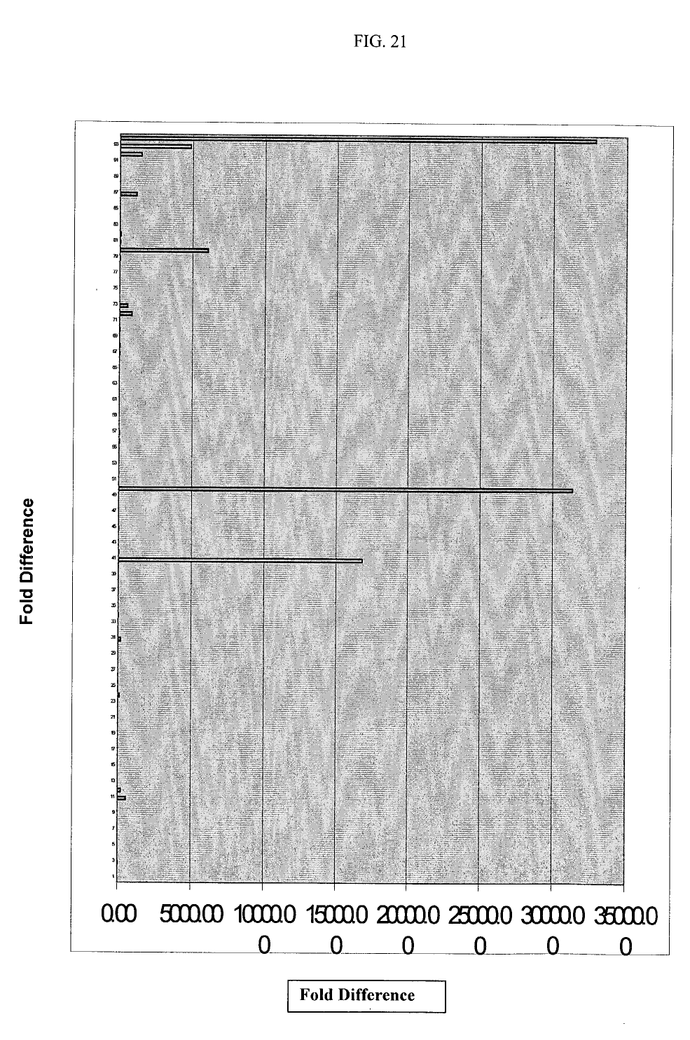

An overall view of the steady state RNA levels amongst all of the cancer cell lines is provided in FIG. 21. Table II (provided below) provides a numerical representation of the values illustrated in FIG. 21. The cooresponding number (“Number”) of each cell line refers to the ‘Y-axis’ of FIG. 21.

Taken together, these results suggest that overexpression of HGPRBMY4 can be involved in the etiology of cancers other than those of the prostate, and that modulators of HGPRBMY4 activity can have utility in the treatment of these cancers.

| | | Fold | |
|---|---|---|---|
| Number | Name | Change | Tissue Origin |
| 1. | A-427 | 1.59 | lung |
| 2. | A-431 | 6.28 | squamous |
| 3. | A2780/DDP-S | 16.17 | ovarian |
| 4. | A2780/DDP-R | 4.99 | ovarian |
| 5. | HCT116/epo5 | 3.99 | colon |
| 6. | A2780/TAX-R | 12.15 | ovarian |
| 7. | A2780/TAX-S | 6.96 | ovarian |
| 8. | A549 | 2.97 | lung |
| 9. | AIN4/myc | 3.23 | breast |
| 10. | AIN4T | 12.35 | breast |
| 11. | AIN4 | 553.27 | breast |
| 12. | BT-549 | 211.49 | breast |
| 13. | BT-20 | 2.10 | breast |
| 14. | C-33A | 1.89 | cervical |
| 15. | CACO-2 | 2.67 | colon |
| 16. | Calu-3 | 3.98 | lung |
| 17. | Calu-6 | 1.00 | lung |
| 18. | BT-474 | 4.09 | breast |
| 19. | CCRF-CEM | 2.97 | leukemia |
| 20. | ChaGo-K-1 | 3.75 | lung |
| 21. | DU4475 | 8.45 | breast |
| 22. | ES-2 | 3.73 | ovarian |
| 23. | H3396 | 2.35 | breast |
| 24. | HBL100 | 107.52 | breast |
| 25. | HCT116/VM46 | 2.73 | colon |
| 26. | HCT116/VP35 | 2.81 | colon |
| 27. | HCT116 | 1.81 | colon |
| 28. | A2780/epo5 | 6.89 | ovarian |
| 29. | HCT116/ras | 1.89 | colon |
| 30. | HCT116/TX15CR | 4.44 | colon |
| 31. | HT-29 | 177.13 | colon |
| 32. | HeLa | 5.11 | cervical |
| 33. | MCF7/Her2 | 5.14 | breast |
| 34. | HL-60 | 20.57 | leukemia |
| 35. | HOC-76 | 3.74 | ovarian |
| 36. | Hs 294T | 4.50 | melanoma |
| 37. | HCT116/vivo | 2.35 | colon |
| 38. | HT-3 | 2.84 | cervical |
| 39. | K-562 | 11.09 | leukemia |
| 40. | SiHa | 6.58 | cervical |
| 41. | LS 174T | 16838.20 | colon |
| 42. | LX-1 | 16.70 | lung |
| 43. | MCF7 | 3.66 | breast |
| 44. | MCF-7/AdrR | 5.42 | breast |
| 45. | MDA-MB-175-VII | 13.74 | breast |
| 46. | MDA-MB-231 | 4.06 | breast |
| 47. | ME-180 | 7.77 | cervical |
| 48. | SK-CO-1 | 7.34 | colon |
| 49. | LoVo | 3.10 | colon |
| 50. | SHP-77 | 31360.03 | lung |
| 51. | DMS 114 | 9.87 | lung |
| 52. | Sk-LU-1 | 2.34 | lung |
| 53. | SK-MES-1 | 2.87 | lung |
| 54. | SW1573 | 5.65 | lung |
| 55. | SW626 | 3.05 | ovarian |
| 56. | SW1271 | 18.46 | lung |
| 57. | SW756 | 17.42 | cervical |
| 58. | SW900 | 5.21 | lung |
| 59. | Colo201 | 6.84 | colon |
| 60. | PC-3 | 5.07 | prostate |
| 61. | OVCAR-3 | 5.38 | ovarian |
| 62. | SW480 | 3.30 | colon |
| 63. | SW620 | 3.07 | colon |
| 64. | PA-1 | 1.56 | ovarian |
| 65. | Caov-3 | 5.08 | ovarian |
| 66. | Ca Ski | 4.89 | cervical |
| 67. | HUVEC | 23.51 | endothelial |
| 68. | Jurkat | 35.44 | leukemia |
| 69. | HS804.SK | 9.23 | skin |
| 70. | WM373 | 19.26 | melanoma |
| 71. | WM852 | 5.16 | melanoma |
| 72. | NCI-N87 | 848.91 | gastric |
| 73. | RPMI-2650 | 537.05 | SCC |
| 74. | SCC-15 | 8.43 | SCC |
| 75. | SCC-4 | 4.58 | SCC |
| 76. | SCC-25 | 3.97 | SCC |
| 77. | SCC-9 | 9.23 | SCC |
| 78. | G-361 | 4.69 | melanoma |
| 79. | C32 | 14.00 | melanoma |
| 80. | A-375 | 6054.00 | melanoma |
| 81. | SK-MEL-1 | 46.87 | melanoma |
| 82. | SK-MEL-28 | 58.62 | melanoma |
| 83. | SK-MEL-5 | 4.66 | melanoma |
| 84. | SK-MEL-3 | 2.29 | melanoma |
| 85. | CA-HPV-10 | 8.57 | prostate |
| 86. | 22Rv1 | 12.24 | prostate |
| 87. | LNCaP-FGC | 1129.24 | prostate |
| 88. | RWPE-1 | 4.10 | prostate |
| 89. | RWPE-2 | 7.82 | prostate |
| 90. | PWR-1E | 12.08 | prostate |
| 91. | DU 145 | 9.90 | prostate |
| 92. | TOTAL RNA, FETAL LUNG | 1462.33 | lung fetal |
| 93. | TOTAL RNA, BREAST | 4852.01 | breast |
| 94. | TOTAL RNA, OVARY | 32847.21 | ovarian |

### Methods

PCR primer pairs were designed to the specific gene and used to measure the steady state levels of mRNA by quantitative PCR across a panel of cell line RNA's. Briefly, first strand CDNA was made from several cell line RNAs and subjected to real time quantitative PCR using a PE 7900HT instrument (Applied Biosystems, Foster City, Calif.) which detects the amount of DNA amplified during each cycle by the fluorescent output of SYBR green, a DNA binding dye specific for double stranded DNA. The specificity of the primer pairs for their targets is verified by performing a thermal denaturation profile at the end of the run which gives an indication of the number of different DNA sequences present by determining melting temperature of double stranded amplicon(s). In the experiment, only one DNA fragment of the correct Tm was detected, having a homogeneous melting point.

Small variations in the amount of cDNA used in each tube was determined by performing parallel experiments using a primer pair for a gene expressed in equal amounts in all tissues, GAPDH. These data were used to normalize the data obtained with the gene specific primer pairs. The PCR data were converted into a relative assessment of the difference in transcript abundance amongst the tissues tested and the data are presented in bar graph form for each transcript.

The formula for calculating the relative abundance is:

Relative abundance=2^{-ΔΔCt}

Where ΔΔCt=(The Ct of the sample−the Ct for cyclophilin)−the Ct for a calibrator sample. The calibrator sample is arbitrarily chosen as the one with the lowest abundance.

For each PCR reaction 10 μL of 2×Sybr Green Master Mix (PE Biosystems) was combined with 4.9 μL water, 0.05 μL of each PCR primer (at 100 micromolar concentration) and 5 microliters of template DNA. The PCR reactions used the following conditions:

95° C. for 10 minutes, then 40 cycles of

95° C. for 30 seconds followed by

60° C. for 1 minute

then the thermal denaturation protocol was begun at 60° C. and the fluorescence measured as the temperature increased slowly to 95° C.

The sequence of the PCR primers were:

| | | |
|---|---|---|
| HGPRBMY4s/ | 5′-ACTGAGCACAGCCTGCATGA-3′ | (SEQ ID NO: 6) |
| GPCR-9s | | |
| HGPRBMY4a/ | 5′-TCTGTAGCAGACAAGCATCAAACTG-3′ | (SEQ ID NO: 7) |
| GPCR-9a | | |

### Example 8

### Expression of HGPRBMY4

### Methods

RNA quantification was performed using the Taqman® real-time-PCR fluorogenic assay. The Taqman® assay is one of the most precise methods for assaying the concentration of nucleic acid templates. All cell lines were grown using standard conditions: RPMI 1640 supplemented with 10% fetal bovine serum, 100 IU/ml penicillin, 100 mg/ml streptomycin, and 2 mM L-glutamine, 10 mM Hepes (all from GibcoBRL). Eighty percent confluent cells were washed twice with phosphate-buffered saline (GibcoBRL) and harvested using 0.25% trypsin (GibcoBRL). RNA was prepared using the RNeasy Maxi Kit from Qiagen. cDNA template for real-time PCR was generated using the Superscript™ First Strand Synthesis system for RT-PCR.

SYBR Green real-time PCR reactions were prepared as follows. The reaction mix consisted of 20 ng first strand cDNA; 50 nM Forward Primer 5′-ACTGAGCACAGCCTGCATGA-3′ (SEQ ID NO: 6); 50 nM Reverse Primer 5′-TCTGTAGCAGACAAGCATCAAACTG-3′ (SEQ ID NO: 7); 0.75×SYBR Green I (Sigma); 1×SYBR Green PCR Buffer (50 mMTris-HCl pH=8.3, 75 mM KCl); 10% DMSO; 3 mM MgCl₂; 300 micromolar each dATP, dGTP, dTTP, dCTP; 1 U Platinum® Taq DNA Polymerase High Fidelity (Life Technologies Cat# 11304-029); 1:50 diluted ROX (Life Technologies). Real-time PCR was performed using an Applied Biosystems 5700 Sequence Detection System. Conditions were 95° C. for 10 min (denaturation and activation of Platinum® Taq DNA Polymerase), 40 cycles of PCR (95° C. for 15 sec, 60° C. for 1 min). PCR products are analyzed for uniform melting using an analysis algorithm built into the 5700 Sequence Detection System.

cDNA quantification used in the normalization of template quantity was performed using Taqman® technology. Taqman® reactions were prepared as follows. The reaction mix consisted of 20 ng first strand cDNA; 25 nM GAPDH-F3, Forward Primer; 250 nM GAPDH-R1 Reverse Primer; 200 nM GAPDH-PVIC Taqman® Probe (fluorescent dye labelled oligonucleotide primer); 1×Buffer A (Applied Biosystems); 5.5 mM MgCl₂; 300 micromolar dATP, dGTP, dTTP, dCTP; 1 U Amplitaq Gold (Applied Biosystems). Real-time PCR was performed using an Applied Biosystems 7700 Sequence Detection System. Conditions for the reaction were 95° C. for 10 min. (denaturation and activation of Amplitaq Gold), 40 cycles of PCR (95° C. for 15 sec, 60° C. for 1 min).

The sequences for the GAPDH oligonucleotides used in the Taqman® reactions were as follows: GAPDH-F3 5′-AGCCGAGCCACATCGCT-3′ (SEQ ID NO: 27) and GAPDH-R1 5′-GTGACCAGGCGCCCAATAC-3′ (SEQ ID NO: 28) with GAPDH-PVIC as the Taqman® Probe-VIC-5′-CAAATCCGTTGACTCCGACCTTCACCTT-3′TAMRA (SEQ ID NO: 29).

The Sequence Detection System generated a Ct (threshold cycle) value that was used to calculate a concentration for each input cDNA template. cDNA levels for each gene of interest were normalized to GAPDH cDNA levels to compensate for variations in total cDNA quantity in the input sample. This was done by generating GAPDH Ct values for each cell line. Ct values for the gene of interest and GAPDH were inserted into the δδCt equation which was used to calculate a GAPDH normalized relative cDNA level for each specific cDNA.

Two plates (OCLP1 and OCLP3) were used for the profiling with partially overlapping samples to allow duplicate results. Cell lines used for OCLP1 are as follows: A431 (squamous origin), LNCAP and PC-3 (prostate); A2780/DDP-S, A2780/epo5, A2780/DDP-R, A2780/TAX-R, ES-2, A2780/TAX-S, UPN251, PA-1, OVCAR-3, SW 626, and Caov-3 (ovarian); Hs 294T (melanoma); SHP-77, A549, LX-1, Sk-LU-1, DMS 114, NCI-H596, MSTO-211H, SW1573, SW900, Calu-3, A-427, ChaGo-K-1, MRC-5, SK-MES-1, Calu-6, and SW1271 (lung); K562, HL-60 and CCRF-CEM (leukemia); HT-1080 (fibrosarcoma); CCD-18Co, LS 174T, SW403, HT-29, T84, MIP, SW480, Colo 320DM, SW837, LS123, HCT116/vivo, CCD-112Co, HCT116/VM46, SW620, HCT116, CCD-33Co, HCT-8, HCT116/TX15CR, LoVo, CACO-2, Cx-1, Colo 205, Colo201, SK-CO-1, HCT116/epo5, HCT116VP35, SW1417, and HCT116/ras (colon); HeLa, SiHa, C-33A, HT-3, Ca Ski, ME-180, and SW756 (cervix); HS 578T, HBL100, BT-474, MDAH 2774, MDA-MB-453, AIN 4T, Her2 MCF-7, T47D, DU4475, H3396, BT-20, MCF7, AIN4/myc, MDA-MB-231, BT-549, AIN 4, MDA-MB-468, BT-483, MCF-7/AdrR, SKBR3, UACC-812, ZR-75-1, and MDA-MB-175-VII (breast).

Cell lines used for OCLP3 are as follows: A-431 (squamous origin); HS804. SK (skin); RPMI-2650, SCC-15, SCC-4, SCC-9, and SCC-25 (head and neck cancer); LNCAP, LNCaP-FGC, 22Rv1, RWPE-1, PWR-1E, CA-HPV-10, DU 145, PC-3, and RWPE-2 (prostate); A2780/DDP-S, A2780/TAX-R, HOC-76, OVCAR-3, A2780/TAX-S, A2780/epo5, Caov-3, SW626, A2780/DDP-R, ES-2, and PA-1 (ovary), SK-MEL-28, WM373, SK-MEL-1, A-375, G-361, WM852, C32, SK-MEL-5, Hs 294T, and SK-MEL-3 (melanoma); SHP-77, LX-1, SW1271, DMS 114, SW900, ChaGo-K-1, Calu-3, SW1573, SK-MES-1, A549, Sk-LU-1, A-427, and Calu-6 (lung); K-562, Jurkat, HL-60, and CCRF-CEM (leukemia); NCI-N87 (gastric); HUVEC (endothelial); LS 174T, HT-29, Colo201, HCT116/ras, SK-CO-1, SW480, LoVo, HCT116/TX15CR, SW620, HCT116/VP35, HCT116/VM46, CACO-2, HCT116/epo5, HCT116/vivo, and HCT116 (colon); Ca Ski, ME-180, HeLa, SiHa, HT-3, SW756, and C-33A (cervix), AIN4, BT-549, HBL100, AIN4T, MCF7/Her2, MCF7, BT-474, MDA-MB-231, DU4475, MCF-7/AdrR, BT-20, H3396, MDA-MB-175-VII, and AIN4/myc (breast). Two additional controls were ovary and fetal lung.

### Expression Results

The GPCR encoding mRNA was expressed highly in several cell lines, with the highest expression in the lung carcinoma line SHP-77, the colon line LS 174T, and prostate LNCAP. Weaker expression was observed in several colon lines (SW403, HT-29, T84, MIP).

Gene profiling (see FIGS. 15 and 16) showed a most remarkable level of high expression in a single prostate tumor compared to control. Similarly, the immunohistochemistry data (see Example 4) showed moderate to strong staining in small subsets of normal prostatic epithelial cells, with most cells staining faintly (five of five samples). In normal tissues, the highest expression is found in blood vessels and associated tissues indicating a possible role in blood flow regulation.

### Example 9

### Signal Transduction Assays

The activity of GPCRs or homologues thereof, can be measured using any assay suitable for the measurement of the activity of a G protein-coupled receptor, as commonly known in the art. Signal transduction activity of a G protein-coupled receptor can be monitor by monitoring intracellular Ca²⁺, cAMP, inositol 1,4,5-triphosphate (IP₃), or 1,2-diacylglycerol (DAG). Assays for the measurement of intracellular Ca²⁺ are described in Sakurai et al. (EP 480 381). Intracellular IP₃ can be measured using a kit available from Amersham, Inc. (Arlington Heights, Ill.). A kit for measuring intracellular cAMP is available from Diagnostic Products, Inc. (Los Angeles, Calif.).

Activation of a G protein-coupled receptor triggers the release of Ca²⁺ ions sequestered in the mitochondria, endoplasmic reticulum, and other cytoplasmic vesicles into the cytoplasm. Fluorescent dyes, for example, fura-2, can be used to measure the concentration of free cytoplasmic Ca²⁺. The ester of fura-2, which is lipophilic and can diffuse across the cell membrane, is added to the media of the host cells expressing GPCRs. Once inside the cell, the fura-2 ester is hydrolyzed by cytosolic esterases to its non-lipophilic form, and then the dye cannot diffuse back out of the cell. The non-lipophilic form of fura-2 will fluoresce when it binds to free Ca²⁺. The fluorescence can be measured without lysing the cells at an excitation spectrum of 340 nm or 380 nm and at fluorescence spectrum of 500 nm (Sakurai et al., EP 480 381).

Upon activation of a G protein-coupled receptor, the rise of free cytosolic Ca²⁺ concentrations is preceded by the hydrolysis of phosphatidylinositol 4,5-bisphosphate. Hydrolysis of this phospholipid by the phospholipase C yields 1,2-diacylglycerol (DAG), which remains in the membrane, and water-soluble inositol 1,4,5-triphosphate (IP₃). Binding of ligands or agonists will increase the concentration of DAG and IP₃. Thus, signal transduction activity can be measured by monitoring the concentration of these hydrolysis products.

To measure the IP₃ concentrations, radioactivity labeled ³H-inositol is added to the media of host cells expressing GPCRs. The ³H-inositol is taken up by the cells and incorporated into IP₃. The resulting inositol triphosphate is separated from the mono and di-phosphate forms and measured (Sakurai et al., EP 480 381). Alternatively, Amersham provides an inositol 1,4,5-triphosphate assay system. With this system Amersham provides tritylated inositol 1,4,5-triphosphate and a receptor capable of distinguishing the radioactive inositol from other inositol phosphates. With these reagents an effective and accurate competition assay can be performed to determine the inositol triphosphate levels.

Cyclic AMP levels can be measured according to the methods described in Gilman et al., Proc. Natl. Acad. Sci. 67:305-312 (1970). In addition, a kit for assaying levels of cAMP is available from Diagnostic Products Corp. (Los Angeles, Calif.).

### Example 10

### GPCR Activity

Another method for screening compounds which are antagonists, and thus inhibit activation of the receptor polypeptide of the present invention is provided. This involves determining inhibition of binding of labeled ligand, such as dATP, dAMP, or UTP, to cells which have the receptor on the surface thereof, or cell membranes containing the receptor. Such a method further involves transfecting a eukaryotic cell with DNA encoding the GPCR polypeptide such that the cell expresses the receptor o n its surface. The cell is then contacted with a potential antagonist in the presence of a labeled form of a ligand, such as dATP, dAMP, or UTP. For example, radioactivity, fluorescence, or any detectable label commonly known in the art can label the ligand. The amount of labeled ligand bound to the receptors is measured by, but not limited to, measuring radioactivity associated with transfected cells or membrane from these cells. If the compound binds to the receptor, the binding of labeled ligand to the receptor is inhibited as determined by a reduction of labeled ligand which binds to the receptors. This method is called a binding assay. Naturally, this same technique can be used to determine agonists.

In a further screening procedure, mammalian cells, for example, but not limited to, CHO, HEK 293, Xenopus Oocytes, RBL-2H3, etc., which are transfected, are used to express the receptor of interest. The cells are loaded with an indicator dye that produces a fluorescent signal when bound to calcium, and the cells are contacted with a test substance and a receptor agonist, such as DATP, DAMP, or UTP. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist or agonist for the receptor.

In yet another screening procedure, mammalian cells are transfected to express the receptor of interest, and are also transfected with a reporter gene construct that is coupled to activation of the receptor (for example, but not limited to luciferase or beta-galactosidase behind an appropriate promoter). The cells are contacted with a test substance and the receptor agonist (ligand), such as dATP, dAMP, or UTP, and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the receptor.

Another screening technique for antagonists or agonists involves introducing RNA encoding the GPCR polypeptide into cells (or CHO, HEK 293, RBL-2H3, etc.) to transiently or stably express the receptor. The receptor cells are then contacted with the receptor ligand, such as dATP, dAMP, or UTP, and a compound to be screened. Inhibition or activation of the receptor is then determined by detection of a signal, such as, cAMP, calcium, proton, or other ions.

### Example 1

### Functional Characterization of HGPRBMY4

### DNA Constructs

The putative GPCR HGPRBMY4 cDNA was PCR amplified using PFUTM (Stratagene). The primers used in the PCR reaction were specific to the HGPRBMY4 polynucleotide and were ordered from Gibco BRL (5 prime primer: 5′-CCCAAGCTTGCACCATGATGGTGGATCCCAATGGCATTG-3′ (SEQ ID NO: 30) 3 prime primer: 5′-GAAGATCTCTAGGGCTCTGAAGCGTGTGTGGCC-3′ (SEQ ID NO: 31). The following 3 prime primer was used to add a Flag-tag epitope to the HGPRBMY4 polypeptide for immunocytochemistry: 5′-GAAGATCTCTACTTGTCGTCGTCGTCCTTGTAGTCCATGGGCTCTGAAGCG TGTGTGGC -3′ (SEQ ID NO: 32). The product from the PCR reaction was isolated from a 0.8% Agarose gel (Invitrogen) and purified using a Gel Extraction Kit™ from Qiagen.

The purified product was then digested overnight with the pcDNA3.1 Hygro™ mammalian expression vector from Invitrogen using the HindIII and BamHI restriction enzymes (New England Biolabs). These digested products were then purified using the Gel Extraction Kit™ from Qiagen and subsequently ligated to the pcDNA3.1 Hygro™ expression vector using a DNA molar ratio of 4 parts insert: 1 vector. All DNA modification enzymes were purchased from NEB. The ligation was incubated overnight at 16° C., after which time, one microliter of the mix was used to transform DH5 alpha cloning efficiency competent E. Coli™ (Gibco BRL). A detailed description of the pcDNA3.1 Hygro™ mammalian expression vector is available at the Invitrogen web site (Hyper Text Transfer Protocol://World Wide Web.Invitrogen.Commercial organization). The plasmid DNA from the ampicillin resistant clones were isolated using the Wizard DNA Miniprep System™ from Promega. Positive clones were then confirmed and scaled up for purification using the Qiagen Maxiprep™ plasmid DNA purification kit.

### Cell Line Generation

The pcDNA3.1hygro vector containing the orphan HGPRBMY4 cDNA was used to transfect CHO/NFAT-CRE or the CHO/NFAT G alpha 15 (Aurora Biosciences) cells using Lipofectamine 2000™ according to the manufacturers specifications (Gibco BRL). Two days later, the cells were split 1:3 into selective media (DMEM 11056, 600 μg/ml Hygromycin, 200 μg/ml Zeocin, 10% FBS). All cell culture reagents were purchased from Gibco BRL-Invitrogen.

The CHO-NFAT/CRE or CHO-NFAT G alpha 15 cell lines, transiently or stably transfected with the orphan HGPRBMY4 GPCR, were analyzed using the FACS Vantage SE™ (BD), fluorescence microscopy (Nikon), and the LJL Analyst™ (Molecular Devices). In this system, changes in real-time gene expression, as a consequence of constitutive G-protein coupling of the orphan HGPRBMY4 GPCR, were examined by analyzing the fluorescence emission of the transformed cells at 447 nm and 518 nm. The changes in gene expression were visualized using Beta-Lactamase as a reporter, and, when induced by the appropriate signaling cascade, hydrolyzed an intracellularly loaded, membrane-permeant ester substrate Cephalosporin-Coumarin-Fluorescein2/Acetoxymethyl (CCF2/AMTM Aurora Biosciences; Zlokarnik, et al., 1998). The CCF2/AMTM substrate is a 7-hydroxycoumarin cephalosporin with a fluorescein attached through a stable thioether linkage. Induced expression of the Beta-Lactamase enzyme was readily apparent since each enzyme molecule produced was capable of changing the fluorescence of many CCF2/AM TM substrate molecules. A schematic of this cell based system is shown below.

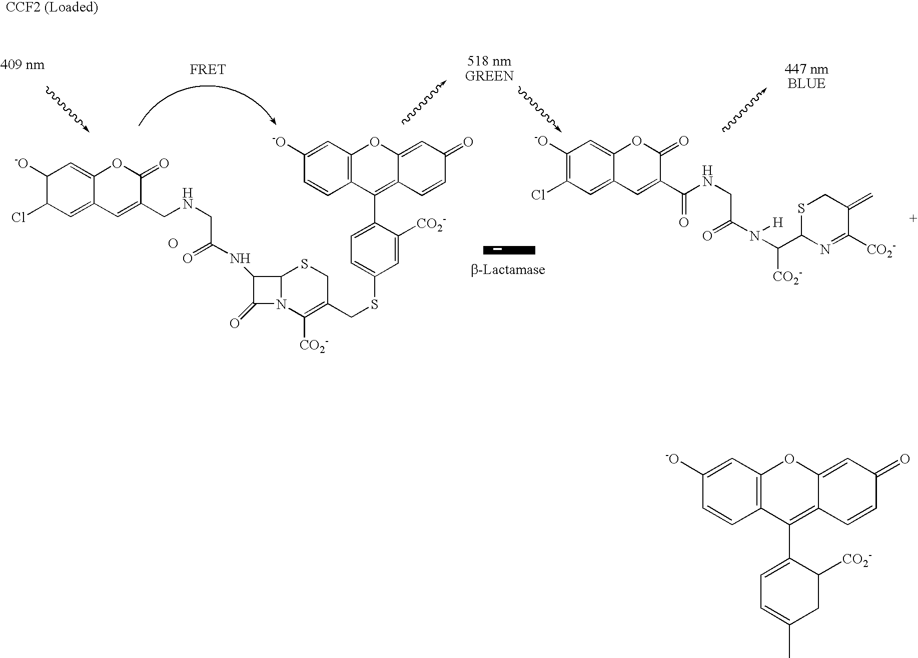

In summary, CCF2/AM TM is a membrane permeant, intracellularly-trapped, fluorescent substrate with a cephalosporin core that links a 7-hydroxycoumarin to a fluorescein. For the intact molecule, excitation of the coumarin at 409 nm results in Fluorescence Resonance Energy Transfer (FRET) to the fluorescein which emits green light at 518 nm. Production of active Beta-Lactamase results in cleavage of the Beta-Lactam ring, leading to disruption of FRET, and excitation of the coumarin only—thus giving rise to blue fluorescent emission at 447 nm.

Fluorescent emissions were detected using a Nikon-TE300 microscope equipped with an excitation filter (D405/10×−25), dichroic reflector (43ODCLP), and a barrier filter for dual DAPI/FITC (510 nM) to visually capture changes in Beta-Lactamase expression. The FACS Vantage SE was equipped with a Coherent Enterprise II Argon Laser and a Coherent 302C Krypton laser. In flow cytometry, UV excitation at 351-364 nm from the Argon Laser or violet excitation at 407 nm from the Krypton laser were used. The optical filters on the FACS Vantage SE are HQ460/50 m and HQ535/40 m bandpass were separated by a 490 dichroic mirror.

Prior to analyzing the fluorescent emissions from the cell lines as described above, the cells were loaded with the CCF2/AM substrate. A 6× CCF2/AM loading buffer was prepared whereby 1 mM CCF2/AM (Aurora Biosciences) was dissolved in 100% DMSO (Sigma). Stock solution (12 μl) was added to 60 μl of 100 mg/ml Pluronic F127 (Sigma) in DMSO containing 0.1% Acetic Acid (Sigma). This solution was added while vortexing to 1 mL of Sort Buffer (PBS minus calcium and magnesium-Gibco-25 mM HEPES-Gibco-pH 7.4, 0.1% BSA). Cells were placed in serum-free media and the 6×CCF2/AM was added to a final concentration of 1×. The cells were then loaded at room temperature for one to two hours, and then subjected to fluorescent emission analysis as described herein. Additional details relative to the cell loading methods and/or instrument settings can be found by reference to the following publications: see Zlokarnik, et al., 1998; Whitney et al., 1998; and BD Biosciences, 1999.

### Immunocytochemistry

The cell lines transfected and selected for expression of Flag-epitope tagged orphan GPCRs were analyzed by immunocytochemistry. The cells were plated at 1×10³ in each well of a glass slide (VWR). The cells were rinsed with PBS followed by acid fixation for 30 minutes at room temperature using a mixture of 5% Glacial Acetic Acid/90% ethanol. The cells were then blocked in 2% BSA and 0.1% Triton in PBS, incubated for 2 h at room temperature or overnight at 4° C. A monoclonal FITC antibody directed against FLAG was diluted at 1:50 in blocking solution and incubated with the cells for 2 h at room temperature. Cells were then washed three times with 0.1% Triton in PBS for five minutes. The slides were overlayed with mounting media dropwise with Biomedia—Gel Mount™ (Biomedia; Containing Anti-Quenching Agent). Cells were examined at 10× magnification using the Nikon TE300 equiped with FI filter (535 nm).

There is strong evidence that certain GPCRs exhibit a cDNA concentration-dependent constitutive activity through cAMP response element (CRE) luciferase reporters (Chen et al., 1999). In an effort to demonstrate functional coupling of HGPRBMY4 to known GPCR second messenger pathways, the HGPRBMY4 polypeptide was expressed at high constitutive levels in the CHO-NFAT/CRE cell line. To this end, the HGPRBMY4 cDNA was PCR amplified and subcloned into the pcDNA3.1 hygro™ mammalian expression vector as described herein. Early passage CHO-NFAT/CRE cells were then transfected with the resulting pcDNA3.1 hygro™/HGPRBMY4 construct. Transfected and non-transfected CHO-NFAT/CRE cells (control) were loaded with the CCF2 substrate and stimulated with 10 nM PMA, and 1 micromolar Thapsigargin (NFAT stimulator) or 10 micromolar Forskolin (CRE stimulator) to fully activate the NFAT/CRE element. The cells were then analyzed for fluorescent emission by Fluorescent Assisted Cell Sorter, FACS.

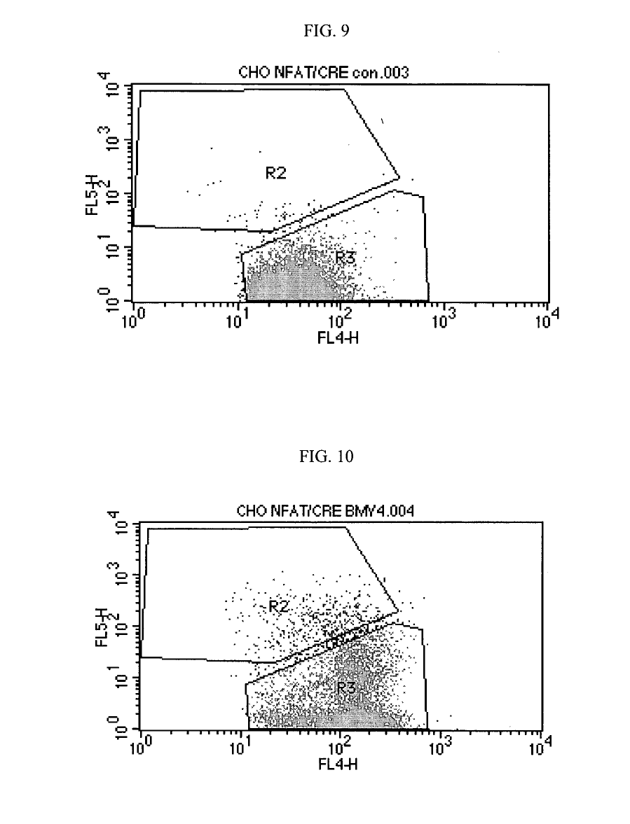

The FACS profile demonstrated the constitutive activity of HGPRBMY4 in the CHO-NFAT/CRE line as evidenced by the significant population of cells with blue fluorescent emission at 447 nm (see FIG. 10: Blue Cells). The cells were analyzed via FACS according to their wavelength emission at 518 nM (Channel R3—Green Cells), and 447 nM (Channel R2—Blue Cells). As shown, overexpression of HGPRBMY4 resulted in functional coupling and subsequent activation of beta lactamase gene expression, as evidenced by the significant number of cells with fluorescent emission at 447 nM relative to the non-transfected control CHO-NFAT/CRE cells (shown in FIG. 9).

As expected, the NFAT/CRE response element in the untransfected control cell line was not activated (i.e., beta lactamase not induced), enabling the CCF2 substrate to remain intact, and resulting in the green fluorescent emission at 518 nM (see FIG. 9—Green Cells). The cells were analyzed via FACS according to their wavelength emission at 518 nM (Channel R3—Green Cells), and 447 nM (Channel R2—Blue Cells). As shown, the vast majority of cells emitted at 518 nM, with minimal emission observed at 447 nM. The latter was expected since the NFAT/CRE response elements remained dormant in the absence of an activated G-protein dependent signal transduction pathway (e.g., pathways mediated by Gq/11 or Gs coupled receptors). As a result, the cell permeant, CCF2/AM TM (Aurora Biosciences; Zlokarnik, et al., 1998) substrate remained intact and emitted light at 518 nM.

A very low level of leaky Beta Lactamase expression was detectable as evidenced by the small population of cells emitting at 447 nm. Analysis of a stable pool of cells transfected with HGPRBMY4 revealed constitutive coupling of the cell population to the NFAT/CRE response element, activation of Beta Lactamase and cleavage of the substrate (FIG. 10—Blue Cells). These results demonstrated that overexpression of HGPRBMY4 leads to constitutive coupling of signaling pathways known to be mediated by Gq/11 or Gs coupled receptors that converge to activate either the NFAT or CRE response elements respectively (Boss et al., 1996; Chen et al., 1999).

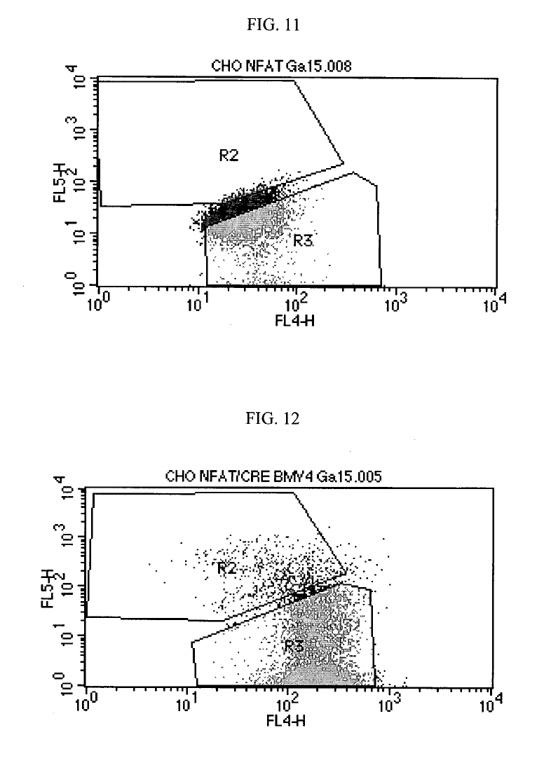

In an effort to further characterize the observed functional coupling of the HGPRBMY4 polypeptide, its ability to couple to a G protein was examined. To this end, the promiscuous G protein, G alpha 15 was utilized. Specific domains of alpha subunits of G proteins have been shown to control coupling to GPCRs (Blahos et al., 2001). It has been shown that the extreme C-terminal 20 amino acids of either G alpha 15 or 16 confer the unique ability of these G proteins to couple to many GPCRs, including those that naturally do not stimulate PLC (Blahos et al., 2001). Indeed, both G alpha 15 and 16 have been shown to couple a wide variety of GPCRs to Phospholipase C activation of calcium mediated signaling pathways (including the NFAT-signaling pathway) (Offermanns & Simon). To demonstrate that HGPRBMY4 was functioning as a GPCR, the CHO-NFAT G alpha 15 cell line that contained only the integrated NFAT response element linked to the Beta-Lactamase reporter was transfected with the pcDNA3.1 hygro™/HGPRBMY4 construct. Analysis of the fluorescence emission from this stable pool showed that HGPRBMY4 constitutively coupled to the NFAT mediated second messenger pathways via G alpha 15 (see FIGS. 11 and 12).

In conclusion, the results were consistent with HGPRBMY4 representing a functional GPCR analogous to known G alpha 15 coupled receptors. Therefore, constitutive expression of HGPRBMY4 in the CHO-NFAT G alpha 15 cell line lead to NFAT activation through accumulation of intracellular Ca²⁺ as has been demonstrated for the M3 muscarinic receptor (Boss et al., 1996).

### Demonstration of Cellular Expression

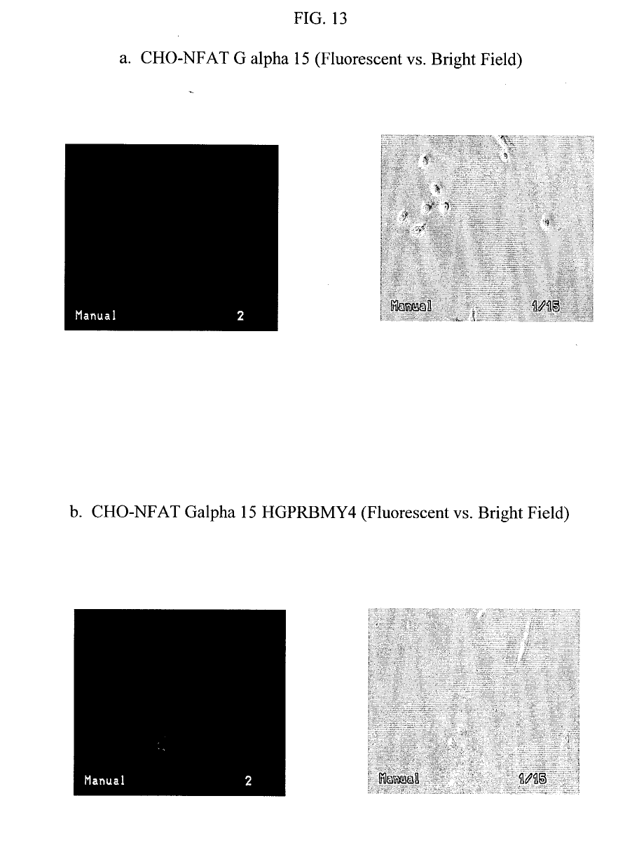

HGPRBMY4 was tagged at the C-terminus using the Flag epitope and inserted into the pcDNA3.1 hygro™ expression vector, as described herein. Immunocytochemistry of CHO-NFAT G alpha 15 cell lines transfected with the Flag-tagged HGPRBMY4 construct with FITC conjugated monoclonal antibody raised against FLAG demonstrated that HGPRBMY4 was indeed a cell surface receptor. The immunocytochemistry also confirmed expression of the HGPRBMY4 in the CHO-NFAT G alpha 15 cell lines. Briefly, CHO-NFAT G alpha 15 cell lines were transfected with pcDNA3.1 hygro™/HGPRBMY4-Flag vector, fixed with 70% methanol, and permeablized with 0.1% Triton X 100. The cells were then blocked with 1% serum and incubated with a FITC conjugated anti Flagmonoclonal antibody at 1:50 dilution in PBS-Triton. The cells were then washed several times with PBS-Triton, overlayed with mounting solution, and fluorescent images were captured (see FIG. 13). FIG. 13 shows the untransfected CHO-NFAT G alpha 15 cell line FACS profile. CHO-NFAT/CRE cell lines transfected with the pcDNA3.1 Hygro™/HGPRBMY4-FLAG mammalian expression vector were subjected to immunocytochemistry using an FITC conjugated monoclonal antibody raised against FLAG, as described herein. Panel A shows the transfected CHO-NFAT/CRE cells under visual wavelengths, and panel B shows the fluorescent emission of the same cells at 530 nm after illumination with a mercury light source. The cellular localization is clearly evident in panel B, and is consistent with the HGPRBMY4 polypeptide representing a member of the GPCR family.

The control cell line, non-transfected CHO-NFAT G alpha 15 cell line, exhibited no detectable background fluorescence (FIG. 13). The BMY4-FLAG tagged expressing CHO-NFAT G alpha 15 line exhibited specific plasma membrane expression as indicated (FIG. 13). These data provided clear evidence that BMY4 was expressed in these cells and the majority of the protein was localized to the cell surface. Cell surface localization was consistent with HGPRBM4 representing a 7 transmembrane domain containing GPCR. Taken together, the data indicated that HGPRBMY4 was a cell surface GPCR that functioned through increases in Ca²⁺ signal transduction pathways via G alpha 15.

### Screening Paradigm

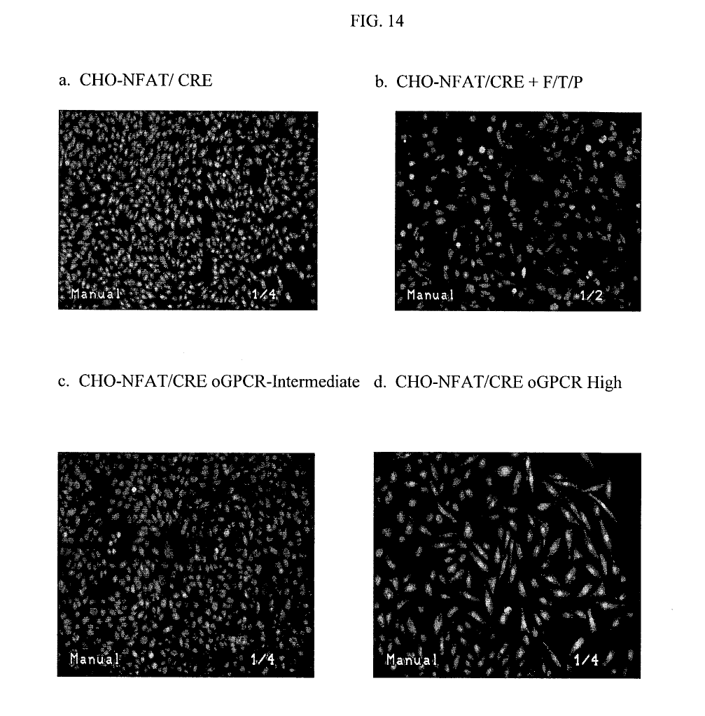

The Aurora Beta-Lactamase technology provided a clear path for identifying agonists and antagonists of the HGPRBMY4 polypeptide. Cell lines that exhibited a range of constitutive coupling activity were identified by sorting through HGPRBMY4 transfected cell lines using the FACS Vantage SE (see FIG. 14). FIG. 14 describes several CHO-NFAT/CRE cell lines transfected with the pcDNA3.1 Hygro™/HGPRBMY4 mammalian expression vector isolated via FACS that had either intermediate or high beta lactamase expression levels of constitutive activation.

For example, cell lines were sorted that had an intermediate level of orphan GPCR expression, which also correlated with an intermediate coupling response, using the LJL analyst. Such cell lines provided the opportunity to screen, indirectly, for both agonists and antogonists of HGPRBMY4 by identifying inhibitors that blocked the beta lactamase response, or agonists that increased the beta lactamase response. As described herein, modulating the expression level of beta lactamase directly correlated with the level of cleaved CCR2 substrate. For example, this screening paradigm was shown to work for the identification of modulators of a known GPCR, 5HT6, that couples through Adenylate Cyclase, in addition to, the identification of modulators of the 5HT2c GPCR, that couples through changes in [Ca²⁺]i. The data shown below represented cell lines that were engineered with the desired pattern of HGPRBMY4 expression to enable the identification of potent small molecule agonists and antagonists. HGPRBMY4 modulator screens can be carried out using a variety of high throughput methods known in the art, though preferably using the fully automated Aurora UHTSS system. The uninduced, orphan-transfected CHO-NFAT/CRE cell line represented the relative background level of beta lactamase expression (FIG. 14; panel a). Following treatment with a cocktail of 10 nanomolar PMA, 1 micromolar Thapsigargin, and 10 micromolar Forskolin (FIG. 14; P/T/F; panel b), the cells fully activated the CRE-NFAT response element demonstrating the dynamic range of the assay. Panel C (FIG. 14) represents an orphan transfected CHO-NFAT/CRE cell line that showed an intermediate level of beta lactamase expression post P/T/F stimulation, while panel D (FIG. 14) represents a HGPRBMY4 transfected CHO-NFAT/CRE cell line that showd a high level of beta lactamase expression post P/T/F stimulation.

FIG. 14 shows that representative transfected CHO-NFAT/CRE cell lines with intermediate and high beta lactamase expression levels were useful in identifing HGPRBMY4 agonists and/or antagonists. Several CHO-NFAT/CRE cell lines transfected with the pcDNA3.1 Hygro™/HGPRBMY4 mammalian expression vector were isolated via FACS that had either intermediate or high beta lactamase expression levels of constitutive activation, as described herein. Panel A (FIG. 14) shows untransfected CHO-NFAT/CRE cells prior to stimulation with 10 nanomolar PMA, 1 micromolar Thapsigargin, and 10 micromolar Forskolin (−P/T/F). Panel B (FIG. 14) shows CHO-NFAT/CRE cells after stimulation with 10 nanomolar PMA,1 micromolar Thapsigargin, and 10 micromolar Forskolin (+P/T/F). Panel C (FIG. 14) shows a representative orphan GPCR (OGPCR) transfected CHO-NFAT/CRE cells that have an intermediate level of beta lactamase expression. Panel D (FIG. 14) shows a representative orphan GPCR transfected CHO-NFAT/CRE that have a high level of beta lactamase expression.

### Example 12

### Phage Display Methods for Identifying Peptide Ligands or Modulayors of Orphan GPCRs

### Library Construction

Two HGPRBMY libraries were used for identifying peptides that can function as modulators. Specifically, a 15-mer library was used to identify peptides that can function as agonists or antagonists. The 15-mer library was an aliquot of the 15-mer library originally constructed by G. P. Smith (Scott, J K and Smith, GP. 1990, Science 249:386-390). A 40-mer library was used for identifying natural ligands and constructed essentially as previously described (B K Kay, et al. 1993, Gene 128:59-65), with the exception that a 15 base pair complementary region was used to anneal the two oligonucleotides, as opposed to 6, 9, or 12 base pairs, as described below.

The oligos used were: Oligo 1: 5′-CGAAGCGTAAGGGCCCAGCCG GCC (NNK×20) CCGGGTCCGGGCGGC-3′ (SEQ ID NO: 46) and Oligo2: 5′-AAAAGGAAAAAAGCGGCCGC (VNN×20) GCCGCCCGGACCCGG-3′ (SEQ ID NO: 47), where N=A, G, C, or T and K=C, G, or T and V=C, A, or G.

The oligos were annealed through their 15 base pair complimentary sequences which encode a constant ProGlyProGlyGly (SEQ ID NO: 48) pentapeptide sequence between the random 20 amino acid segments, and then extended by standard procedure using Klenow enzyme. This was followed by endonuclease digestion using Sfi1 and Not1 enzymes and ligation to Sfi1 and Not1 cleaved pCantab5E (Pharmacia). The ligation mixture was electroporated into E. coli XL1Blue and phage clones were essentially generated as suggested by the manufacturer for making ScFv antibody libraries in pCantab5E.

### Sequencing Bound Phage

Standard procedures commonly known in the art were used. Phage in eluates were infected into E. coli host strain (TG1 for the 15-mer library; XL1 Blue for the 40-mer library) and plated for single colonies. Colonies were grown in liquid and sequenced by standard procedure which involved: 1) generating PCR product with suitable primers of the library segments in the phage genome (15 mer library) or pCantab5E (40 mer library); and 2) sequencing PCR products using one primer of each PCR primer pair. Sequences were visually inspected or by using the Vector NTI alignment tool.

### Peptide Modulators

The following serve as non-limiting examples of peptides:

| | |
|---|---|
| GDFWYEACESSCAFW | (SEQ ID NO: 53) |
| CLRSGTGCAFQLYRF | (SEQ ID NO: 54) |
| FAGQIIWYDALDTLM | (SEQ ID NO: 55) |
| LIFFDARDCCFNEQL | (SEQ ID NO: 56) |
| LEWGSDVFYDVYDCC | (SEQ ID NO: 57) |
| RIVPNGYFNVHGRSL | (SEQ ID NO: 58) |
| WERSSAGCADQQYRC | (SEQ ID NO: 59) |
| YFSDGESFFEPGDCC | (SEQ ID NO: 60) |

### Peptide Synthesis

Peptides were synthesized on Fmoc-Knorr amide resin [N-(9-fluorenyl)methoxycarbonyl-Knorr amide-resin; Midwest Biotech; Fishers, IN] with an Applied Biosystems (Foster City, Calif.) model 433A synthesizer and the FastMoc chemistry protocol (0.25 mmol scale) supplied with the instrument. Amino acids were double coupled as their N-α-Fmoc-derivatives and reactive side chains were protected as follows: Asp, Glu: t-Butyl ester (OtBu); Ser, Thr, Tyr: t-Butyl ether (tBu); Asn, Cys, Gln, His: Triphenylmethyl (Trt); Lys, Trp: t-Butyloxycarbonyl (Boc); Arg: 2,2,4,6,7-Pentamethyldihydrobenzofuran-5-sulfonyl (Pbf). After the final double coupling cycle, the N-terminal Fmoc group was removed by the multi-step treatment with piperidine in N-Methylpyrrolidone described by the manufacturer. The N-terminal free amines were then treated with 10% acetic anhydride, 5% Diisopropylamine in N-Methylpyrrolidone to yield the N-acetyl-derivative. The protected peptidyl-resins were simultaneously deprotected and removed from the resin by standard methods. The lyophilized peptides were purified on C₁₈ to apparent homogeneity as judged by RP-HPLC analysis. Predicted peptide molecular weights were verified by electrospray mass spectrometry (J. Biol. Chem. 273:12041-12046, 1998).

Cyclic analogs were prepared from the crude linear products. The cysteine disulfide was formed using one of the following methods:

### Method 1

A sample of the crude peptide was dissolved in water at a concentration of 0.5 mg/mL and the pH adjusted to 8.5 with NH₄OH. The reaction was stirred at room temperature, and monitored by RP-HPLC. Once completed, the reaction was adjusted to pH 4 with acetic acid and lyophilized. The product was purified and characterized as above.

### Method 2

A sample of the crude peptide was dissolved at a concentration of 0.5mg/mL in 5% acetic acid. The pH was adjusted to 6.0 with NH₄OH. DMSO (20% by volume) was added and the reaction was stirred overnight. After analytical RP-HPLC analysis, the reaction was diluted with water and triple lyophilized to remove DMSO. The crude product was purified by preparative RP-HPLC (JACS. 113:6657, 1991)

### Assessing Effect of Peptides on GPCR Function

The effect of any one of these peptides on the function of the GPCR of the present invention was determined by adding an effective amount of each peptide to each functional assay. Representative functional assays are described more specifically herein, particularly Example 7.

### Uses of the Peptide Modulators of the Present Invention

The aforementioned peptides of the present invention can be useful for a variety of purposes, though most notably for modulating the function of the GPCR of the present invention, and potentially with other GPCRs of the same G-protein coupled receptor subclass (e.g., peptide receptors, adrenergic receptors, purinergic receptors, etc.), and/or other subclasses known in the art. For example, the peptide modulators of the present invention can be useful as HGPRBMY4 agonists. Alternatively, the peptide modulators of the present invention can be useful as HGPRBMY4 antagonists of the present invention. In addition, the peptide modulators of the present invention can be useful as competitive inhibitors of the HGPRBMY4 cognate ligand(s), or can be useful as non-competitive inhibitors of the HGPRBMY4 cognate ligand(s).

Furthermore, the peptide modulators of the present invention can be useful in assays designed to either deorphan the HGPRBMY4 polypeptide of the present invention, or to identify other agonists or antagonists of the HGPRBMY4 polypeptide of the present invention, particularly small molecule modulators.

### Example 13

### Method of Creating N- and C-terminal Deletion Mutants of the HGPRBMY4 Polypetide

As described elsewhere herein, the present invention encompasses the creation of N- and C-terminal deletion mutants, in addition to any combination of N- and C-terminal deletions thereof, of the HGPRBMY4 polypeptide of the present invention. A number of methods are available to one skilled in the art for creating such mutants. Such methods can include a combination of PCR amplification and gene cloning methodology. Although one of skill in the art of molecular biology, through the use of the teachings provided or referenced herein, and/or otherwise known in the art as standard methods, could readily create each deletion mutants of the present invention, exemplary methods are described below.

Briefly, using the isolated cDNA clone encoding the full-length HGPRBMY4 polypeptide sequence, appropriate primers of about 15-25 nucleotides derived from the desired 5′ and 3′ positions of SEQ ID NO: 1 can be designed to PCR amplify, and subsequently clone, the intended N- and/or C-terminal deletion mutant. Such primers could comprise, for example, an initiation and stop codon for the 5′ and 3′ primer, respectively. Such primers can also comprise restriction sites to facilitate cloning of the deletion mutant post amplification. Moreover, the primers can comprise additional sequences, such as, for example, flag-tag sequences, kozac sequences, or other sequences discussed and/or referenced herein.

For example, in the case of the Q27 to P318 N-terminal deletion mutant, the following primers could be used to amplify a cDNA fragment of this deletion mutant:

| | | |
|---|---|---|
| 5′ | 5′-GCAGCA GCGGCCGC CAGTTCTGGTTGGCCTTCCCATTG-3′ | (SEQ ID NO: 49) |
| Primer | NotI | |
| 3′ | 5′-GCAGCA GTCGAC GGGCTCTGAAGCGTGTGTGGCCAC-3′ | (SEQ ID NO: 50) |
| Primer | SalI | |

For example, in the case of the M1 to K297 C-terminal deletion mutant, the following primers could be used to amplify a cDNA fragment of this deletion mutant:

| | | |
|---|---|---|
| 5′ | 5′-GCAGCA GCGGCCGC ATGATGGTGGATCCCAATGGCAATG-3′ | (SEQ ID NO: 51) |
| Primer | NotI | |
| 3′ | 5′-GCAGCA GTCGAC CTTCACTCCATAGACAATTGGGTTG-3′ | (SEQ ID NO: 52) |
| Primer | SalI | |

Representative PCR amplification conditions are provided below, although the skilled artisan would appreciate that other conditions can be required for efficient amplification. A 100 microliter PCR reaction mixture can be prepared using 10 ng of the template DNA (cDNA clone of HGPRBMY4), 200 micromolar 4dNTPs, 1 micromolar primers, 0.25U Taq DNA polymerase (PE), and standard Taq DNA polymerase buffer. Typical PCR cycling condition are as follows:

20-25 cycles: 45 sec, 93° C.

2 min, 50° C.

2 min, 72° C.

1 cycle: 10 min, 72° C.

5 After the final extension step of PCR, 5U Klenow Fragment can be added and incubated for 15 min at 30° C.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and can be desirable in certain circumstances. The digested fragment and vector are then ligated using a DNA ligase, and then used to transform competent E. coli cells using methods provided herein and/or otherwise known in the art.

The 5′ primer sequence for amplifying any additional N-terminal deletion mutants can be determined by reference to the following formula:

(S+(X*3)) to ((S+(X*3))+25),

wherein ‘S’ is equal to the nucleotide position of the initiating start codon of the HGPRBMY4 gene (SEQ ID NO: 1), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer sense strand of SEQ ID NO: 1. Once the corresponding nucleotide positions of the primer are determined, the final nucleotide sequence can be created by the addition of applicable restriction site sequences to the 5′ end of the sequence, for example. As referenced herein, the addition of other sequences to the 5′ primer can be desired in certain circumstances (e.g., kozac sequences, etc.).

The 3′ primer sequence for amplifying any additional N-terminal deletion mutants can be determined by reference to the following formula:

(S+(X*3)) to ((S+(X*3))−25),

wherein ‘S’ is equal to the nucleotide position of the initiating start codon of the HGPRBMY4 gene (SEQ ID NO: 1), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer anti-sense strand of SEQ ID NO: 1. Once the corresponding nucleotide positions of the primer are determined, the final nucleotide sequence can be created by the addition of applicable restriction site sequences to the 5′ end of the sequence, for example. As referenced herein, the addition of other sequences to the 3′ primer can be desired in certain circumstances (e.g., stop codon sequences, etc.). The skilled artisan would appreciate that modifications of the above nucleotide positions can be necessary for optimizing PCR amplification.

The same general formulas provided above can be used in identifying the 5′ and 3′ primer sequences for amplifying any C-terminal deletion mutant of the present invention. Moreover, the same general formulas provided above can be used in identifying the 5′ and 3′ primer sequences for amplifying any combination of N-terminal and C-terminal deletion mutant of the present invention. The skilled artisan would appreciate that modifications of the above nucleotide positions can be necessary for optimizing PCR amplification.

In preferred embodiments, the following N-terminal HGPRBMY4 deletion polypeptides are encompassed by the present invention (of SEQ ID NO: 2): M1-P318, M2-P318, V3-P318, D4-P318, P5-P318, N6-P318, G7-P318, N8-P318, E9-P318, S10-P318, S11-P318, A12-P318, T13-P318, Y14-P318, F15-P318, I16-P318, L17-P318, I18-P318, G19-P318, L20-P318, P21-P318, G22-P318, L23-P318, E24-P318, E25-P318, A26-P318, Q27-P318, F28-P318, W29-P318, L30-P318, A31-P318, F32-P318, P33-P318, L34-P318, C35-P318, S36-P318, L37-P318, Y38-P318, L39-P318, I40-P318, A41-P318, V42-P318, L43-P318, G44-P318, N45-P318, L46-P318, T47-P318, I48-P318, I49-P318, Y50-P318, I51-P318, V52-P318, R53-P318, T54-P318, E55-P318, H56-P318, S57-P318, L58-P318, H59-P318, E60-P318, P61-P318, M62-P318, Y63-P318, I64-P318, F65-P318, L66-P318, C67-P318, M68-P318, L69-P318, S70-P318, G71-P318, I72-P318, D73-P318, I74-P318, L75-P318, I76-P318, S77-P318, T78-P318, S79-P318, S80-P318, M81-P318, P82-P318, K83-P318, M84-P318, L85-P318, A86-P318, I87-P318, F88-P318, W89-P318, F90-P318, N91-P318, S92-P318, T93-P318, T94-P318, I95-P318, Q96-P318, F97-P318, D98-P318, A99-P318, C100-P318, L101-P318, L102-P318, Q103-P318, M104-P318, F105-P318, A106-P318, I107-P318, H108-P318, S109-P318, L110-P318, S111-P318, G112-P318, M113-P318, E114-P318, S115-P318, T116-P318, V117-P318, L118-P318, L119-P318, A120-P318, M121-P318, A122-P318, F123-P318, D124-P318, R125-P318, Y126-P318, V127-P318, A128-P318, I129-P318, C130-P318, H131-P318, P132-P318, L133-P318, R134-P318, H135-P318, A136-P318, T137-P318, V138-P318, L139-P318, T140-P318, L141-P318, P142-P318, R143-P318, V144-P318, T145-P318, K146-P318, I147-P318, G148-P318, V149-P318, A150-P318, A151-P318, V152-P318, V153-P318, R154-P318, G155-P318, A156-P318, A157-P318, L158-P318, M159-P318, A160-P318, P161-P318, L162-P318, P163-P318, V164-P318, F165-P318, I166-P318, K167-P318, Q168-P318, L169-P318, P170-P318, F171-P318, C172-P318, R173-P318, S174-P318, N175-P318, I176-P318, L177-P318, S178-P318, H179-P318, S180-P318, Y181-P318, C182-P318, L183-P318, H184-P318, Q185-P318, D186-P318, V187-P318, M188-P318, K189-P318, L190-P318, A191-P318, C192-P318, D193-P318, D194-P318, I195-P318, R196-P318, V197-P318, N198-P318, V199-P318, V200-P318, Y201-P318, G202-P318, L203-P318, I204-P318, V205-P318, I206-P318, I207-P318, S208-P318, A209-P318, I210-P318, G211-P318, L212-P318, D213-P318, S214-P318, L215-P318, L216-P318, I217-P318, S218-P318, F219-P318, S220-P318, Y221-P318, L222-P318, L223-P318, I224-P318, L225-P318, K226-P318, T227-P318, V228-P318, L229-P318, G230-P318, L231-P318, T2342-P318, R233-P318, E234-P318, A235-P318, Q236-P318, A237-P318, K238-P318, A239-P318, F240-P318, G241-P318, T242-P318, C243-P318, V244-P318, S245-P318, H246-P318, V247-P318, C248-P318, A249-P318, V250-P318, F251-P318, I252-P318, F253-P318, Y254-P318, V255-P318, P256-P318, F257-P318, I258-P318, G259-P318, L260-P318, S261-P318, M262-P318, V263-P318, H264-P318, R265-P318, F266-P318, S267-P318, K268-P318, R269-P318, R270-P318, D271-P318, S272-P318, P273-P318, L274-P318, P275-P318, V276-P318, I277-P318, L278-P318, A279-P318, N280-P318, I281-P318, Y282-P318, L283-P318, L284-P318, V285-P318, P286-P318, P287-P318, V288-P318, L289-P318, N290-P318, P291-P318, I292-P318, V293-P318, Y294-P318, G295-P318, V296-P318, K297-P318, T298-P318, K299-P318, E300-P318, I301-P318, R302-P318, Q303-P318, R304-P318, I305-P318, L306-P318, R307-P318, L308-P318, F309-P318, H310-P318, V311-P318, and/or A312-P318 of SEQ ID NO: 2. Polynucleotide sequences encoding these polypeptides are also included in SEQ ID NO: 1. The present invention also encompasses the use of these N-terminal HGPRBMY4 deletion polypeptides as immunogenic and/or antigenic epitopes as described elsewhere herein.

In preferred embodiments, the following C-terminal HGPRBMY4 deletion polypeptides are encompassed by the present invention (of SEQ ID NO: 2): M1-P318, M1-E317, M1-S316, M1-A315, M1-H314, M1-T313, M1-A312, M1-V311, M1-H310, M1-F309, M1-L308, M1-R307, M1-L306, M1-I305, M1-R304, M1-Q303, M1-R302, M1-I301, M1-E300, M1-K299, M1-T298, M1-K297, M1-V296, M1-G295, M1-Y294, M1-V293, M1-I292, M1-P291, M1-N290, M1-L289, M1-V288, M1-P287, M1-P286, M1-V285, M1-L284, M1-L283, M1-Y282, M1-281, M1-N280, M1-A279, M1-L278, M1-I277, M1-V276, M1-P275, M1-L274, M1-P273, M1-S272, M1-D271, M1-R270, M1-R269, M1-K268, M1-S267, M1-F266, M1-R265, M1-H264, M1-V263, M1-M262, M1-S261, M1-L260, M1-G259, M1-I258, M1-F257, M1-P256, M1-V255, M1-Y254, M1-F253, M1-I252, M1-F251, M1-V250, M1-A249, M1-C248, M1-V247, M1-H246, M1-S245, M1-V244, M1-C243, M1-T242, M1-G241, M1-F240, M1-A239, M1-K238, M1-A237, M1-Q236, M1-A235, M1-E234, M1-R233, M1-T232, M1-L231, M1-G230, M1-L229, M1-V228, M1-T227, M1-K226, M1-L225, M1-I224, M1-L223, M1-L222, M1-Y221, M1-S220, M1-F219, M1-S218, M1-I217, M1-L216, M1-L215, M1-S214, M1-D213, M1-L212, M1-G211, M1-I210, M1-A209, M1-S208, M1-I207, M1-I206, M1-V205, M1-I204, M1-L203, M1-G202, M1-Y201, M1-V200, M1-V199, M1-N198, M1-V197, M1-R196, M1-I195, M1-D194, M1-D193, M1-C192, M1-A191, M1-L190, M1-K189, M1-M188, M1-V187, M1-D186, M1-Q185, M1-H184, M1-L183, M1-C182, M1-Y181, M1-S180, M1-H179, M1-S178, M1-L177, M1-I176, M1-N175, M1-S174, M1-R173, M1-C172, M1-F171, M1-P170, M1-L169, M1-Q168, M1-K167, M1-I166, M1-F165, M1-V164, M1-P163, M1-L162, M1-P161, M1-A160, M1-M159, M1-L158, M1-A157, M1-A156, M1-G155, M1-R154, M1-V153, M1-V152, M1-A151, M1-A150, M1-V149, M1-G148, M1-I147, M1-K146, M1-T145, M1-V144, M1-R143, M1-P142, M1-L141, M1-T140, M1-L139, M1-V138, M1-T137, M1-A136, M12-H135, M1-R134, M1-L133, M1-P132, M1-H131, M1-C130, M1-1129, M1-A128, M1-V127, M1-Y126, M1-R125, M1-D124, M1-F123, M1-A122, M1-M121, M1-A120, M1-L119, M1-L118, M1-V117, M1-T116, M1-S115, M1-E114, M1-M113, M1-G112, M1-S111, M1-L110, M1-S109, M1-H108, M1-I107, M1-A106, M1-F105, M1-M104, M1-Q103, M1-L102, M1-L101, M1-C100, M1-A99, M1-D98, M1-F97, M1-Q96, M1-195, M1-T94, M1-T93, M1-S92, M1-N91, M1-F90, M1-W89, M1-F88, M1-I87, M1-A86, M1-L85, M1-M84, M1-K83, M1-P82, M1-M81, M1-S80, M1-S79, M1-T78, M1-S77, M1-I76, M1-L75, M1-I74, M1-D73, M1-I72, M1-G71, M1-S70, M1-L69, M1-M68, M1-C67, M1-L66, M1-F65, M1-I64, M1-Y63, M1-M62, M1-P61, M1-E60, M1-H59, M1-L58, M1-S57, M1-H56, M1-E55, M1-T54, M1-R53, M1-V52, M1-I51, M1-Y50, M1-I49, M1-I48, M1-T47, M1-L46, M1-N45, M1-G44, M1-L43,M1-V42, M1-A41, M1-I40, M1-L39, M1-Y38, M1-L37, M1-S36, M1-C35, M1-L34, M1-P33, M1-F32, M1-A31, M1-L30, M1-W29, M1-F28, M1-Q27, M1-A26, M1-E25, M1-E24, M1-L23, M1-G22, M1-P21, M1-L20, M1-G19, M1-I18, M1-L17, M1-I16, M1-F15, M1-Y14, M1-T13, M1-A12, M1-S11, M1-S10, M1-E9, M1-N8, and/or M1-G7 of SEQ ID NO: 2. Polynucleotide sequences encoding these polypeptides are also included in SEQ ID NO: 1. The present invention also encompasses the use of these C-terminal HGPRBMY4 deletion polypeptides as immunogenic and/or antigenic epitopes as described elsewhere herein.

Alternatively, preferred polypeptides of the present invention can comprise polypeptide sequences having, for example, internal regions of the HGPRBMY4 polypeptide (e.g., any combination of both N- and C-terminal HGPRBMY4 polypeptide deletions) of SEQ ID NO: 2. For example, internal regions could be defined by the equation: amino acid NX to amino acid CX, wherein NX refers to any N-terminal deletion polypeptide amino acid of HGPRBMY4 (SEQ ID NO: 2), and where CX refers to any C-terminal deletion polypeptide amino acid of HGPRBMY4 (SEQ ID NO: 2). Polynucleotides encoding these polypeptides are also included in SEQ ID NO: 1. The present invention also encompasses the use of these polypeptides as an immunogenic and/or antigenic epitope as described elsewhere herein.

### Example 14

### Method of Enhancing the Biological Activity or Functional Characteristics through Molecular Evolution

Although many of the most biologically active proteins known are highly effective for their specified function in an organism, they often possess characteristics that make them undesirable for transgenic, therapeutic, pharmaceutical, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement can, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein can also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered G-protein coupled receptor can be constitutively active upon binding of its cognate ligand. Alternatively, an engineered G-protein coupled receptor can be constitutively active in the absence of ligand binding. In yet another example, an engineered GPCR can be capable of being activated with less than all of the regulatory factors and/or conditions typically required for GPCR activation (e.g., ligand binding, phosphorylation, conformational changes, etc.). Such GPCRs would be useful in screens to identify GPCR modulators, among other uses described herein.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides having specific regions of interest (as described by Derbyshire, K. M. et al, Gene, 46:145-152, (1986), and Hill, D E, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis can counter or inhibit the desired benefit of a useful mutation.

While both of the aforementioned methods are effective for creating randomized pools of macromolecule variants, a third method, termed “DNA Shuffling,” or “sexual PCR” (W P C, Stemmer, Proc. Natl. Acad. Sci., 91:10747, (1994)) has recently been elucidated. DNA shuffling has also been referred to as “directed molecular evolution,” “exon-shuffling,” “directed enzyme evolution,” “in vitro evolution,” and “artificial evolution.” Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also can hybridize to other DNA fragments having different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments -further diversifying the potential hybridation sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly, prepare the DNA substrate to be subjected to the DNA shuffling reaction. Preparation can be in the form of simply purifying the DNA from contaminating cellular material, chemicals, buffers, oligonucleotide primers, deoxynucleotides, RNAs, etc., and can entail the use of DNA purification kits as those provided by Qiagen, Inc. or by the Promega, Corp., for example.

Once the DNA substrate has been purified, it would be subjected to Dnase I digestion. About 2-4 micrograms of the DNA substrate(s) would be digested with 0.0015 units of Dnase I (Sigma) per ul in 100 microliters of 50 mM Tris-HCl, pH 7.4/1 mM MgCl₂ for 10-20 min. at room temperature. The resulting fragments of 10-50 base pairs could then be purified by running them through a 2% low-melting point agarose gel by electrophoresis onto DE81 ion-exchange paper (Whatman) or could be purified using Microcon concentrators (Amicon) of the appropriate molecular weight cuttoff, or could use oligonucleotide purification columns (Qiagen), in addition to other methods known in the art. If using DE81 ion-exchange paper, the 10-50 base pair fragments could be eluted from said paper using 1 M NaCl, followed by ethanol precipitation.

The resulting purified fragments would then be subjected to a PCR assembly reaction by re-suspension in a PCR mixture containing: 2 mM of each dNTP, 2.2 mM MgCl₂, 50 mM KCl, 10 mM Tris-HCl, pH 9.0, and 0.1% Triton X-100, at a final fragment concentration of 10-30 nanograms/microliter. No primers are added at this point. Taq DNA polymerase (Promega) would be used at 2.5 units per 100 microliters of reaction mixture. A PCR program of 94 C for 60 s; 94 C for 30 s, 50 ° C.-55 C for 30 s, and 72 C for 30 s using 30-45 cycles, followed by 72 C for 5 min. using an MJ Research (Cambridge, Mass.) PTC-150 thermocycler. After the assembly reaction is completed, a 1:40 dilution of the resulting primerless product would then be introduced into a PCR mixture (using the same buffer mixture used for the assembly reaction) containing 0.8 micromolar of each primer and subjecting this mixture to 15 cycles of PCR (using 94 C for 30 s, 50 C for 30 s, and 72 C for 30 s). The referred primers would be primers having the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailered to the desired level of mutagenesis using the methods described by Zhao, et al. (Nucl. Acid Res., 25(6):1307-1308, (1997).

As described above, once the randomized pool has been created, it can then be subjected to a specific screen to identify the variant possessing the desired characteristic(s). Once the variant has been identified, DNA having the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology can be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16,000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there can be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host. For example, a particular varient of the present invention can be created and isolated using DNA shuffling technology. Such a variant can have all of the desired characteristics, though can be highly immunogenic in a host due to its novel intrinsic structure. Specifically, the desired characteristic can cause the polypeptide to have a non-native strucuture which could no longer be recognized as a “self” molecule, but rather as a “foreign,” and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel varient that provided the desired characteristics.

Likewise, the invention encompasses the application of DNA shuffling technology to the evolution of polynucletotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homolog sequences, additional homologous sequences, additional non-homologous sequences, sequences from another species, and any number and combination of the above.

In addition to the described methods above, there are a number of related methods that can also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, can be found in U.S. Pat. No. 5,605,793; PCT Application No. WO 95/22625; PCT Application No. WO 97/20078; PCT Application No. WO 97/35966; and PCT Application No. WO 98/42832; PCT Application No. WO 00/09727 specifically provides methods for applying DNA shuffling to the identification of herbicide selective crops which could be applied to the polynucleotides and polypeptides of the present invention; additionally, PCT Application No. WO 00/12680 provides methods and compositions for generating, modifying, adapting, and optimizing polynucleotide sequences that confer detectable phenotypic properties on plant species; each of the above are hereby incorporated in their entirety herein for all purposes.

### Example 15

### Antisense Oligonucleotide Method and the NFkB Pathway

Antisense molecules or nucleic acid sequences complementary to the HGPRBMY4 protein-encoding sequence, or any part thereof, was used to decrease or to inhibit the expression of naturally occurring HGPRBMY4. Although the use of antisense or complementary oligonucleotides comprising about 15 to 35 base-pairs is described, essentially the same procedure was used with smaller or larger nucleic acid sequence fragments. An oligonucleotide based on the coding sequence of HGPRBMY4 protein,as shown in FIG. 1, or as depicted in SEQ ID NO: 1, for example, was used to inhibit expression of naturally occurring HGPRBMY4. The complementary oligonucleotide was typically designed from the most unique 5′ sequence and was used either to inhibit transcription by preventing promoter binding to the coding sequence, or to inhibit translation by preventing the ribosome from binding to the HGPRBMY4 protein-encoding transcript, among others. However, other regions can also be targeted.

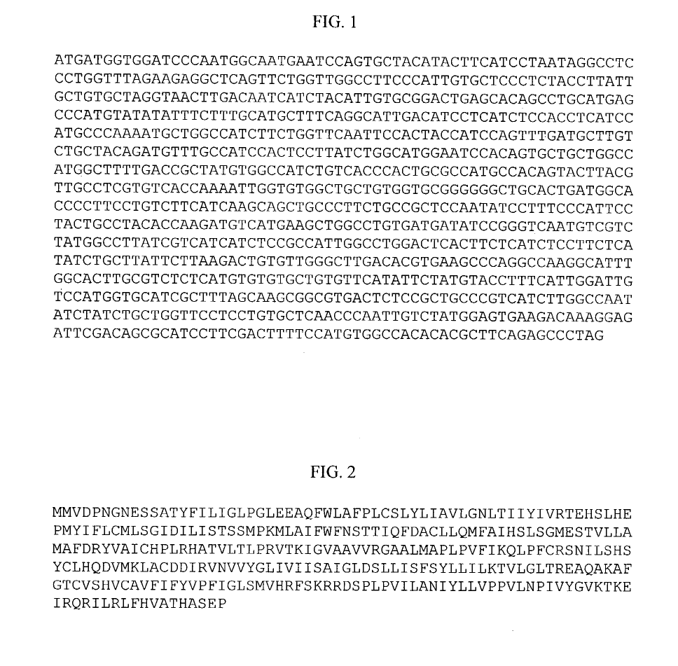

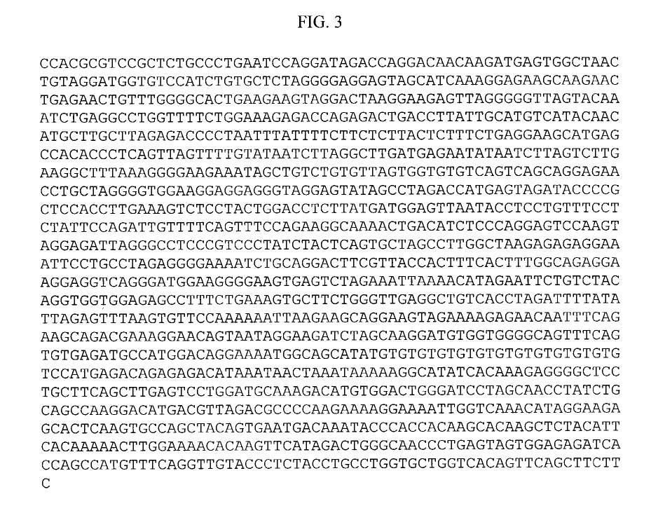

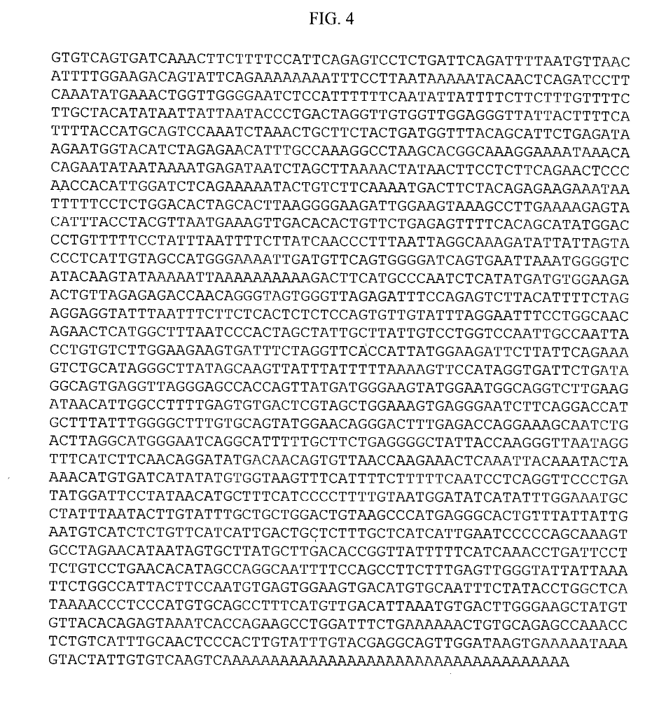

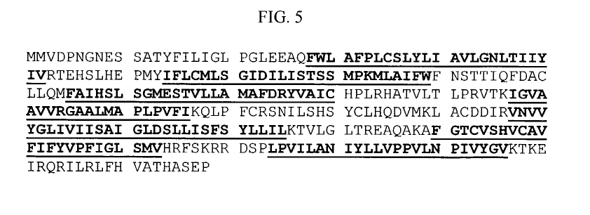

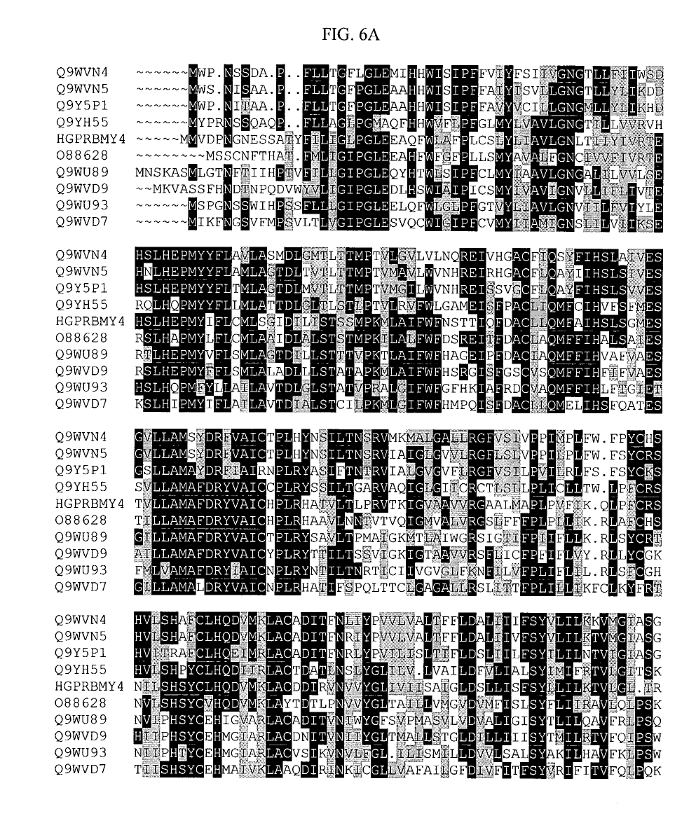

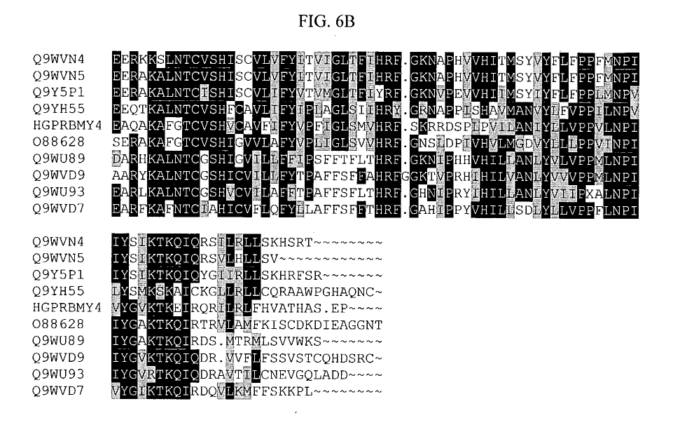

Using an appropriate portion of a 5′ sequence of SEQ ID NO: 1, an effective antisense oligonucleotide included any of about 15-35 nucleotides spanning the region which translates into the signal or 5′ coding sequence, among other regions, of the polypeptide as shown in FIG. 2 (SEQ ID NO: 2). Appropriate oligonucleotides were designed using OLIGO 4.06 software (National Biosciences Inc.; Plymouth, Minn.) and the HGPRBMY4 protein coding sequence (SEQ ID NO: 1). Preferred oligonucleotides are deoxynucleotide, or chimeric deoxynucleotide/ribonucleotide based and are provided below. The oligonucleotides were synthesized using chemistry essentially as described in U.S. Pat. No. 5,849,902; which is hereby incorporated herein by reference in its entirety.

Five antisense oligonucleotide sequences used for identifying E-selectin/NFkB phenotype for HGPRBMY4 are as follows:

SEQUENCE

| | |
|---|---|
| 5′-GGUCUAGGCUAUACUCCUACCCUCC-3′ | (SEQ ID NO: 65) |
| 5′-GGACACCAUCCUACAGUUAGCCACU-3′ | (SEQ ID NO: 66) |
| 5′-CCUCCUUCCUCUGCCAAAGUGAAAG-3′ | (SEQ ID NO: 67) |
| 5′-CCUGUCCAUGGCAUCUCACACUGAA-3′ | (SEQ ID NO: 68) |
| 5′-CCAGGCCUCAGAUUUGUACUAACCC-3′ | (SEQ ID NO: 69) |

The HGPRBMY4 polypeptide has been shown to be involved in the regulation of mammalian NFkB and apoptosis pathways. Subjecting cells to an effective amount of a pool of all five of the above antisense oligoncleotides resulted in a significant increase in E-selectin expression and activity in HMVEC cells providing convincing evidence that HGPRBMY4 at least regulated the activity and/or expression of E-selectin either directly or indirectly. Moreover, the results suggested that HGPRBMY4 was involved in the negative regulation of NFkB/IkB alpha activity and expression, either directly or indirectly. The NFkB/E-selectin assay used is described below. This assay was based upon the analysis of E-selectin activity as a downstream marker for inflammatory or proliferative signal transduction events.

### Day 0

Plates were coated with collagen. For one plate, collagen was stored at 4° C. at 0.4 mg/ml until needed. Glacial acetic acid (112.5 microliters) was added to 13.5 ml of H₂O, and then 84.35 microliters of collagen was added to 13.5 ml of acetic acid. The mixture (250 microliters) was added to each well and incubated for 2 hr at room temperature for a final concentration of 2.5 microgram/ml). Collagen was removed and rinsed twice with 500 microliters of PBS. Media (200 microliters) was added and kept at 37° C. until ready for use. HMVEC cells were then plated at 30,000 cells/well in 48 well plates.

### Day 1

HMVEC cells were transfected using 1 microgram/ml Lipofectamine 2000 lipid and 25 nM antisense oligonucleotide according to the following protocol. The necessary materials were: HMVEC cells maintained in EBM-2 (Clonetics) supplemented with EGM-2 MV (Clonetics), Opti-MEM (Gibco-BRL), Lipofectamine 2000 (Invitrogen), antisense oligomers (Sequitur), polystyrene tubes, and tissue culture-treated plates.

A 10× stock of Lipofectamine 2000 (10 micrograms/ml is 10×) was prepared, and the diluted lipid was allowed to stand at room temperature for 15 minutes. Stock solution of Lipofectamine 2000 was 1 mg/ml. A 10× solution for transfection was 10 micrograms/ml. To prepare 10× solution, 10 microliters of Lipofectamine 2000 stock was diluted per 1 ml of Opti-MEM (serum free media).

A 10× stock of each oligomer to be used in the transfection was then prepared. Stock solutions of oligomers were at 100 micromolar in 20 mM HEPES, pH 7.5. 10× concentration of oligomer was 0.25 micromolar. To prepare the 10× solutions, 2.5 microliters of oligomer was diluted per 1 ml of Opti-MEM.

Equal volumes of the 10× Lipofectamine 2000 stock and the 10× oligomer solutions were mixed well and incubated for 15 minutes at RT to allow the oligomer and lipid to complex. The resulting mixture was 533 . After incubating 15 minutes to allow the complex to form, 4 volumes of full growth media were added to the oligomer/lipid complexes (solution is now 1×). The media was then aspirated from the cells, and 0.5 ml of the 1× oligomer/lipid complexes was added to each well.

The cells were incubated for 16-24 hours at 37° C. in a humidified CO₂ incubator. Oligomer update was evaluated by fluorescent microscopy. In addition, the cell viability was evaluated by performing dead stain analysis.

### Day 2

### TNF Stimulation

TNF was stored at −70° C. in 10 microliter aliquots at a concentration of 50 micrograms/ml. Two fold dilutions of TNF were made by first adding 10 microliters to 1 ml to give 500 ng/ml of the TNF aliquots. Then 300 microliters was added to 15 ml to give 10 ng/ml. The final solution (250 microliters) was added to each well and the cells were stimulated for 6 hours at 37° C.

After stimulation, 100 microliters of supernatant was removed from each well and stored at −70° C. The remaining media was then removed from each well. The cells were then titered. Fresh media (200 microliters) was added to each well. CTR (50 microliters; cell titer reagent) was added to each well. Two blank wells were included for controls with media alone and CTR. The cells were incubated at 37° C. for about 90 minutes. One hundred microliters were removed from each well and moved to a 96 well plate. The absorbance was then read at 490 nm on spectrophotometer.

During the 90 minute incubation, a glutaraldehyde solution was prepared. Glutaraldehyde (140 microliters) was added to 14 ml PBS (0.5% glutaraldehyde). Blocking buffer was also prepared. For one plate, 50 ml was made by combining 46.5 ml PBS, 1.5 ml goat serum, and 2 ml 0.5M EDTA.

Once the cell titer was complete, the remaining media was removed and 250 microliters glutaraldehyde solution was added to each well, and incubated for 10 minutes at 4° C. The plates were then agitated and 500 microliters blocking buffer was added to each well. The plates were then incubated at 4° C. overnight.

### Day 3

### E-selectin Solution Preparation

Stock (22.5 microliters of 100 micrograms/ml) was added to 9 ml blocking buffer. The mixture (150 microliters) was added to each well and incubated for 1hour at 37° C. The wells were washed 4 times with cold PBS; the plates were agitated between washes and then aspirated after completion to remove remaining PBS.

HRP was prepared by adding 2.25 microliters HRP to 9 ml blocking buffer. The mixture (150 microliters) was added to each well and incubated for 1 hour at 37° C. The wells were washed 4 times with cold PBS; plates were agitated between washes and then aspirated at the end to remove any remaining PBS. Peroxidase color reagent (150 microliters) was added to each well for development. The plates were allowed to develop for about 5 minutes and stopped with 150 microliters 1N H₂SO₄. One hundred microliters per well were then transferred from each well to a 96 well plate and the OD was read at 450 nm.

The positive samples were then noted. It was expected that at least one or more of the NFkB associated polynucleotides and polypeptides of the present invention would show a positive result in this assay. Any positive results would provide convincing evidence that the sequences were involved in the NFkB pathway, either directly or indirectly. Specifically, HGPRBMY4 resulted in inhibition of E-selectin expression in HMVEC cells in the above assay.

### Example 16

### Method of Screening, In vitro, Compounds that Bind to the HGPRBMY4 Polypetide

In vitro systems can be designed to identify compounds capable of binding the HGPRBMY4 polypeptide of the invention. Compounds identified can be useful, for example, in modulating the activity of wild type and/or mutant HGPRBMY4 polypeptide, preferably mutant HGPRBMY4 polypeptide, can be useful in elaborating the biological function of the HGPRBMY4 polypeptide, can be utilized in screens for identifying compounds that disrupt normal HGPRBMY4 polypeptide interactions, or can in themselves disrupt such interactions.

The principle of the assays used to identify compounds that bind to the HGPRBMY4 polypeptide involves preparing a reaction mixture of the HGPRBMY4 polypeptide and the test compound under conditions and for a time sufficient to allow the two components to interact and bind, thus forming a complex which can be removed and/or detected in the reaction mixture. These assays can be conducted in a variety of ways. For example, one method to conduct such an assay would involve anchoring HGPRBMY4 polypeptide or the test substance onto a solid phase and detecting HGPRBMY4 polypeptide/test compound complexes anchored on the solid phase at the end of the reaction. In one embodiment of such a method, the HGPRBMY4 polypeptide can be anchored onto a solid surface, and the test compound, which is not anchored, can be labeled, either directly or indirectly.

In practice, microtitre plates can conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously immobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the immobilized component (the antibody, in turn, can be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for HGPRBMY4 polypeptide or the test compound to anchor any complexes formed in solution, and a labeled antibody specific for the other component of the possible complex to detect anchored complexes.

Another example of a screening assay to identify compounds that bind to HGPRBMY4, relates to the application of a cell membrane-based scintillation proximity assay (“SPA”). Such an assay would require the idenification of a ligand for HGPRBMY4 polypeptide. Once identified, unlabeled ligand is added to assay-ready plates that would serve as a positive control. The SPA beads and membranes are added next, and then ²⁵I-labeled ligand is added. After an equilibration period of 2-4 hours at room temperature, the plates can be counted in a scintillation counting machine, and the percent inhibition or stimulation calculated. Such an SPA assay may be based upon a manual, automated, or semi-automated platform, and encompass 96, 384, 1536-well plates or more. Any number of SPA beads may be used as applicable to each assay. Examples of SPA beads include, for example, Leadseeker WGA PS (Amersham cat # RPNQ 0260), and SPA Beads (PVT-PEI-WGA-TypeA; Amersham cat # RPNQ0003). The utilized membranes may also be derived from a number of cell line and tissue sources depending upon the expression profile of the respective polypeptide and the adaptability of such a cell line or tissue source to the development of a SPA-based assay. Examples of membrane preparations include, for example, cell lines transformed to express the receptor to be assayed in CHO cells or HEK cells, for example. SPA-based assays are well known in the art and are encompassed by the present invention. One such assay is described in U.S. Pat. No. 4,568,649, which is incorporated herein by reference. The skilled artisan would acknowledge that certain modifications of known SPA assays may be required to adapt such assays to each respective polypeptide.

One such screening procedure involves the use of melanophores which are transfected to express the HGPRBMY4 polypeptide of the present invention. Such a screening technique is described in PCT WO 92/01810, published Feb. 6, 1992. Such an assay may be employed to screen for a compound which inhibits activation of the receptor polypeptide of the present invention by contacting the melanophore cells which encode the receptor with both the receptor ligand, such as LPA, and a compound to be screened. Inhibition of the signal generated by the ligand indicates that a compound is a potential antagonist for the receptor, i.e., inhibits activation of the receptor.

The technique may also be employed for screening of compounds which activate the receptor by contacting such cells with compounds to be screened and determining whether such compound generates a signal, i.e., activates the receptor. Other screening techniques include the use of cells which express the HGPRBMY4 polypeptide (for example, transfected CHO cells) in a system which measures extracellular pH changes caused by receptor activation. In this technique, compounds may be contacted with cells expressing the receptor polypeptide of the present invention. A second messenger response, e.g., signal transduction or pH changes, is then measured to determine whether the potential compound activates or inhibits the receptor.

Another screening technique involves expressing the HGPRBMY4 polypeptide in which the receptor is linked to phospholipase C or D. Representative examples of such cells include, but are not limited to, endothelial cells, smooth muscle cells, and embryonic kidney cells. The screening may be accomplished as hereinabove described by detecting activation of the receptor or inhibition of activation of the receptor from the phospholipase second signal.

Another method involves screening for compounds which are antagonists or agonists by determining inhibition of binding of labeled ligand, such as LPA, to cells which have the receptor on the surface thereof, or cell membranes containing the receptor. Such a method involves transfecting a cell (such as eukaryotic cell) with DNA encoding the HGPRBMY4 polypeptide such that the cell expresses the receptor on its surface. The cell is then contacted with a potential antagonist or agonist in the presence of a labeled form of a ligand, such as LPA. The ligand can be labeled, e.g., by radioactivity. The amount of labeled ligand bound to the receptors is measured, e.g., by measuring radioactivity associated with transfected cells or membrane from these cells. If the compound binds to the receptor, the binding of labeled ligand to the receptor is inhibited as determined by a reduction of labeled ligand which binds to the receptors. This method is called binding assay.

Another screening procedure involves the use of mammalian cells (CHO, HEK 293, Xenopus Oocytes, RBL-2H3, etc) which are transfected to express the receptor of interest. The cells are loaded with an indicator dye that produces a fluorescent signal when bound to calcium, and the cells are contacted with a test substance and a receptor agonist, such as LPA. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist or agonist for the receptor.

Another screening procedure involves use of mammalian cells (CHO, HEK293, Xenopus Oocytes, RBL-2H3, etc.) which are transfected to express the receptor of interest, and which are also transfected with a reporter gene construct that is coupled to activation of the receptor (for example, luciferase or beta-galactosidase behind an appropriate promoter). The cells are contacted with a test substance and the receptor agonist (ligand), such as LPA, and the signal produced by the reporter gene is measured after a defined period of time. The signal can be measured using a luminometer, spectrophotometer, fluorimeter, or other such instrument appropriate for the specific reporter construct used. Change of the signal generated by the ligand indicates that a compound is a potential antagonist or agonist for the receptor.

Another screening technique for antagonists or agonits involves introducing RNA encoding the HGPRBMY4 polypeptide into Xenopus oocytes (or CHO, HEK 293, RBL-2H3, etc.) to transiently or stably express the receptor. The receptor oocytes are then contacted with the receptor ligand, such as LPA, and a compound to be screened. Inhibition or activation of the receptor is then determined by detection of a signal, such as, cAMP, calcium, proton, or other ions.

Another method involves screening for HGPRBMY4 polypeptide inhibitors by determining inhibition or stimulation of HGPRBMY4 polypeptide-mediated cAMP and/or adenylate cyclase accumulation or dimunition. Such a method involves transiently or stably transfecting a eukaryotic cell with HGPRBMY4 polypeptide receptor to express the receptor on the cell surface.

The cell is then exposed to potential antagonists or agonists in the presence of HGPRBMY4 polypeptide ligand, such as LPA. The changes in levels of cAMP is then measured over a defined period of time, for example, by radio-immuno or protein binding assays (for example using Flashplates or a scintillation proximity assay). Changes in cAMP levels can also be determined by directly measuring the activity of the enzyme, adenylyl cyclase, in broken cell preparations. If the potential antagonist or agonist binds the receptor, and thus inhibits HGPRBMY4 polypeptide-ligand binding, the levels of HGPRBMY4 polypeptide-mediated cAMP, or adenylate cyclase activity, will be reduced or increased.

One preferred screening method involves co-transfecting HEK-293 cells with a mammalian expression plasmid encoding a G-protein coupled receptor (GPCR), such as HGPRBMY4, along with a mixture comprised of mammalian expression plasmids cDNAs encoding GU15 (Wilkie T. M. et al Proc Natl Acad Sci USA 1991 88: 10049-10053), GU16 (Amatruda T. T. et al Proc Natl Acad Sci USA 1991 8: 5587-5591, and three chimeric G-proteins refered to as Gqi5, Gqs5, and Gqo5 (Conklin B R et al Nature 1993 363: 274-276, Conklin B. R. et al Mol Pharmacol 1996 50: 885-890). Following a 24 h incubation the trasfected HEK-293 cells are plated into poly-D-lysine coated 96 well black/clear plates (Becton Dickinson, Bedford, Mass.).

The cells are assayed on FLFPR (Fluorescent Imaging Plate Reader, Molecular Devices, Sunnyvale, Calif.) for a calcium mobilization response following addition of test ligands. Upon identification of a ligand which stimulates calcium mobilization in HEK-293 cells expressing a given GPCR and the G-protein mixtures, subsequent experiments are performed to determine which, if any, G-protein is required for the functional response. HEK-293 cells are then transfected with the test GPCR, or co-transfected with the test GPCR and G015, GD16, GqiS, Gqs5, or Gqo5. If the GPCR requires the presence of one of the G-proteins for functional expression in HEK-293 cells, all subsequent experiments are performed with HEK-293 cell cotransfected with the GPCR and the G-protein which gives the best response. Alternatively, the receptor can be expressed in a different cell line, for example RBL-2H3, without additional Gproteins.

Another screening method for agonists and antagonists relies on the endogenous pheromone response pathway in the yeast, Saccharomyces cerevisiae. Heterothallic strains of yeast can exist in two mitotically stable haploid mating types, MATa and MATa. Each cell type secretes a small peptide hormone that binds to a G-protein coupled receptor on opposite mating type cells which triggers a MAP kinase cascade leading to G1 arrest as a prelude to cell fusion.

Genetic alteration of certain genes in the pheromone response pathway can alter the normal response to pheromone, and heterologous expression and coupling of human G-protein coupled receptors and humanized G-protein subunits in yeast cells devoid of endogenous pheromone receptors can be linked to downstream signaling pathways and reporter genes (e.g., U. S. Pat. Nos. 5,063,154; 5,482,835; 5,691,188). Such genetic alterations include, but are not limited to, (i) deletion of the STE2 or STE3 gene encoding the endogenous G-protein coupled pheromone receptors; (ii) deletion of the FAR1 gene encoding a protein that normally associates with cyclindependent kinases leading to cell cycle arrest; and (iii) construction of reporter genes fused to the FUS 1 gene promoter (where FUS 1 encodes a membrane-anchored glycoprotein required for cell fusion). Downstream reporter genes can permit either a positive growth selection (e.g., histidine prototrophy using the FUS1-HIS3 reporter), or a calorimetric, fluorimetric or spectrophotometric readout, depending on the specific reporter construct used (e.g., b-galactosidase induction using a FUS1-LacZ reporter).

The yeast cells can be further engineered to express and secrete small peptides from random peptide libraries, some of which can permit autocrine activation of heterologously expressed human (or mammalian) G-protein coupled receptors (Broach, J. R. and Thorner, J., Nature 384: 14-16, 1996; Manfredi et al., Mol. Cell. Biol. 16: 4700-4709,1996). This provides a rapid direct growth selection (e.g, using the FUS 1-HIS3 reporter) for surrogate peptide agonists that activate characterized or orphan receptors. Alternatively, yeast cells that functionally express human (or mammalian) G-protein coupled receptors linked to a reporter gene readout (e.g., FUS1-LacZ) can be used as a platform for high-throughput screening of known ligands, fractions of biological extracts and libraries of chemical compounds for either natural or surrogate ligands.

Functional agonists of sufficient potency (whether natural or surrogate) can be used as screening tools in yeast cell-based assays for identifying G-protein coupled receptor antagonists. For example, agonists will promote growth of a cell with FUS-HIS3 reporter or give positive readout for a cell with FUSI-LacZ. However, a candidate compound which inhibits growth or negates the positive readout induced by an agonist is an antagonist. For this purpose, the yeast system offers advantages over mammalian expression systems due to its ease of utility and null receptor background (lack of endogenous G-protein coupled receptors) which often interferes with the ability to identify agonists or antagonists.

The contents of all patents, patent applications, published PCT applications and articles, books, references, reference manuals and abstracts cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

### References

1. Rees, S., Brown, S., Stables, J.: “Reporter gene systems for the study of G Protein Coupled Receptor signalling in mammalian cells.” In Milligan G. (ed.): Signal Transduction: A practical approach. Oxford: Oxford University Press, 1999: 171-221.

2. Alam, J., Cook, J. L.: “Reporter Genes: Application to the study of mammalian gene transcription.” Anal. Biochem. 1990; 188: 245-254.

3. Selbie, L. A. and Hill, S. J.: “G protein-coupled receptor cross-talk: The fine-tuning of multiple receptor-signaling pathways.” TiPs. 1998; 19: 87-93.

4. Boss, V., Talpade, D. J., and Murphy, T. J.: “Induction of NFAT mediated transcription by Gq-coupled Receptors in lympoid and non-lymphoid cells.” J. Biol. Chem. 1996; 271: 10429-10432.

5. George, S. E., Bungay, B. J., and Naylor, L. H.: “Functional coupling of endogenous serotonin (5-HT1B) and calcitonin (C1a) receptors in CHO cells to a cyclic AMP-responsive luciferase reporter gene.” J. Neurochem. 1997; 69: 1278-1285.

6. Suto, C M, Igna D M: “Selection of an optimal reporter for cell-based high throughput screening assays.” J. Biomol. Screening. 1997; 2: 7-12.

7. Zlokarnik, G., Negulescu, P. A., Knapp, T. E., More, L., Burres, N., Feng, L., Whitney, M., Roemer, K., and Tsien, R. Y. “Quantitation of transcription and clonal selection of single living cells with a B-Lactamase Reporter.” Science. 1998; 279: 84-88.

8. Fiering et. al., Genes Dev. 4, 1823 (1990).

9. Karttunen and N. Shastri, Proc. Natl Acad. Sci. 88, 3972 (1991).

10. Hawes, B. E., Luttrell. L. M., van Biesen, T., and Lefkowitz, R. J. (1996) J. Biol. Chem. 271, 12133-12136.

11. Gilman, A. G. (1987) Annul. Rev. Biochem. 56, 615-649.

12. T. Maniatis et al., Molecular Cloning: A Laboratory Manual, Cold Spring Harbor Laboratory, Cold Spring Harbor, N.Y., 1989.

13. Salcedo, R., Ponce, M. L., Young, H. A., Wasserman, K., Ward, J. M., Kleinman, H. K., Oppenheim, J. J., Murphy, W. J. “Human endothelial cells express CCF2 and respond to MCP-1: direct role of MCP-1 in angiogenesis and tumor progression.” Blood. 2000; 96 (1): 34-40.

14. Sica, A., Saccani, A., Bottazzi, B., Bernasconi, S., Allavena, P., Gaetano, B., LaRossa, G., Scotton, C., Balkwill F., Mantovani, A. “Defective expression of the monocyte chemotactic protein 1 receptor CCF2 in macrophages associated with human ovarian carcinoma.” J. Immunol. 2000; 164: 733-8.

15. Kypson, A., Hendrickson, S., Akhter, S., Wilson, K., McDonald, P., Lilly, R., Dolber, P., Glower, D., Lefkowitz, R., Koch, W. “Adenovirus-mediated gene transfer of the B2 AR to donor hearts enhances cardiac function.” Gene Therapy. 1999; 6: 1298-304.

16. Dorn, G. W., Tepe, N. M., Lorenz, J. N., Kock, W. J., Ligget, S. B. “Low and high level transgenic expression of B2AR differentially affect cardiac hypertrophy and function in Galpha q-overexpressing mice.” Proc. Natl. Acad. Sci. 1999; 96: 6400-5.

17. J. Wess. “G protein coupled receptor: molecular mechanisms involved in receptor activation and selectivity of G-protein recognition.” FASEB. 1997; 11:346-354.

18. Whitney, M, Rockenstein, E, Cantin, G., Knapp, T., Zlokarnik, G., Sanders, P., Durick, K., Craig, F. F., and Negulescu, P. A. “A genome-wide functional assay of signal transduction in living mammalian cells.” Nature Biotech. 1998; 16: 1329-1333.

19. B D Biosciences: FACS Vantage SE Training Manual. Part Number 11-11020-00 Rev. A. August 1999.

20. Chen, G., Jaywickreme, C., Way, J., Armour S., Queen K., Watson., C., Ignar, D., Chen, W. J., Kenakin, T. “Constitutive Receptor systems for drug discovery.” J. Pharmacol. Toxicol. Methods 1999; 42: 199-206.

21. Blahos, J., Fischer,T., Brabet, I., Stauffer,D., Rovelli, G., Bockaert, J., and Pin, J.-P. “A novel Site on the G alpha-protein that Rocognized Heptahelical Receptors.” J.Biol. Chem. 2001; 275, No. 5, 3262-69.

22. Offermanns, S. & Simon, M. I. “G alpha 15 and G alpha 16 Couple a Wide Variety of Receptors to Phospholipase C.” J. Biol. Chem. 1995; 270, No. 25, 15175-80.

What is claimed is:

1. An isolated nucleic acid molecule consisting of a polynucleotide having a nucleotide sequence selected from the group consisting of:
(a) a polynucleotide fragment of SEQ ID NO:1 or a polynucleotide fragment of the cDNA sequence included in ATCC Deposit No:PTA-2682, which is hybridizable to SEQ ID NO:1;
(b) a polynucleotide encoding a polypeptide fragment of SEQ ID NO:2 or a polypeptide fragment encoded by the cDNA sequence included in ATCC Deposit No:PTA-2682, which is hybridizable to SEQ ID NO:1;
(c) a polynucleotide encoding a polypeptide domain of SEQ ID NO:2 or a polypeptide domain encoded by the cDNA sequence included in ATCC Deposit No:PTA-2682, which is hybridizable to SEQ ID NO:1;
(d) a polynucleotide encoding a polypeptide epitope of SEQ ID NO:2 or a polypeptide epitope encoded by the cDNA sequence included in ATCC Deposit No:PTA-2682, which is hybridizable to SEQ ID NO:1;
(e) a polynucleotide encoding a polypeptide of SEQ ID NO:2 or the cDNA sequence included in ATCC Deposit No:PTA-2682, which is hybridizable to SEQ ID NO:1, having biological activity;
(f) a polynucleotide which is a variant of SEQ ID NO:1;
(g) a polynucleotide which is an allelic variant of SEQ ID NO:1;
(h) a polynucleotide which encodes a species homologue of the SEQ ID NO:2;
(i) a polynucleotide which represents the complimentary sequence (antisense) of SEQ ID NO:1;
(j) a polynucleotide corresponding to nucleotides 4 to 954 of SEQ ID NO:1;
(k) a polynucleotide corresponding to nucleotides I to 954 of SEQ ID NO: 1; or
(l) a polynucleotide capable of hybridizing under stringent conditions to any one of the polynucleotides specified in (a)-(k), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding a G-protein coupled receptor protein.

3. The isolated nucleic acid molecule of claim 1, wherein the polynucleotide fragment comprises a nucleotide sequence encoding the sequence identified as SEQ ID NO:2 or the polypeptide encoded by the cDNA sequence included in ATCC Deposit No:PTA-2682, which is hybridizable to SEQ ID NO: 1.

4. A recombinant vector comprising the isolated nucleic acid molecule of claim 1.

5. A method of making a recombinant host cell comprising the isolated nucleic acid molecule of claim 1.

6. A recombinant host cell produced by the method of claim 5.

7. The recombinant host cell of claim 6 comprising vector sequences.

8. An isolated polypeptide comprising an amino acid sequence at least 95% identical to a sequence selected from the group consisting of:
(a) a polypeptide fragment of SEQ ID NO:2 or the encoded sequence included in ATCC Deposit No:PTA-2682;
(b) a polypeptide fragment of SEQ ID NO:2 or the encoded sequence included in ATCC Deposit No:PTA-2682, having biological activity;
(c) a polypeptide domain of SEQ ID NO:2 or the encoded sequence included in ATCC Deposit No:PTA-2682;
(d) a polypeptide epitope of SEQ ID NO:2 or the encoded sequence included in ATCC Deposit No:PTA-2682;
(e) a full length protein of SEQ ID NO:2 or the encoded sequence included in ATCC Deposit No:PTA-2682;
(f) a variant of SEQ ID NO:2;
(g) an allelic variant of SEQ ID NO:2;
(h) a species homologue of SEQ ID NO:2;
(i) a polypeptide corresponding to amino acids 1 to 318 of SEQ ID NO:2; and
(j) a polypeptide corresponding to amino acids 2 to 318 of SEQ ID NO:2.

9. An isolated antibody that binds specifically to the isolated polypeptide of claim 8.

10. A recombinant host cell that expresses the isolated polypeptide of claim 8.

11. A method of making an isolated polypeptide comprising:
(a) culturing the recombinant host cell of claim 10 under conditions such that said polypeptide is expressed; and
(b) recovering said polypeptide.

12. A polypeptide produced by claim 11.

13. A method for preventing, treating, or ameliorating a medical condition, comprising administering to a mammalian subject a therapeutically effective amount of the polypeptide of claim 8 or a modulator thereof.

14. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

15. A method of diagnosing a pathological condition or a susceptibility to a pathological condition in a subject comprising:
(a) determining the presence or amount of expression of the polypeptide of claim 8 in a biological sample; and
(b) diagnosing a pathological condition or a susceptibility to a pathological condition based on the presence or amount of expression of the polypeptide.

16. The method of diagnosing a pathological condition of claim 15 wherein the condition is a member of the group consisting of: a reproductive disorder; a male reproductive disorder; a prostate disorder; prostate cancer; proliferative condition of the prostate; cardiovascular disorder; heart disorder; pulmonary disorder; lung disorder; lung cancer; proliferative condition of the lung; gastrointestinal disorder; a colon disorder; colon cancer; female reproductive disorder; ovarian cancer; placental disorder; proliferative condition of the ovary; melanoma; vascular disorders; umbilical cord disorder; disorders associated with aberrant E-selectin expression or activity; disorders associated with aberrant NFkB expression or activity; disorders associated with aberrant IkBalpha expression or activity; an inflammatory disorder; an inflammatory disorder associated with abberant NFkB regulation or regulation of the NFkB pathway; and a proliferative disorder associated with abberant NFkB regulation or regulation of the NFkB pathway.

17. A method for treating, or ameliorating a medical condition with the polypeptide provided as SEQ ID NO:2, or a modulator thereof, wherein the medical condition is a member of the group consisting of: a reproductive disorder; a male reproductive disorder; a prostate disorder; prostate cancer; proliferative condition of the prostate; cardiovascular disorder; heart disorder; pulmonary disorder; lung disorder; lung cancer; proliferative condition of the lung; gastrointestinal disorder; a colon disorder; colon cancer; female reproductive disorder; ovarian cancer; placental disorder; proliferative condition of the ovary; melanoma; vascular disorders; umbilical cord disorder; disorders associated with aberrant E-selectin expression or activity; disorders associated with aberrant NFkB expression or activity; disorders associated with aberrant IkBalpha expression or activity; an inflammatory disorder; an inflammatory disorder associated with abberant NFkB regulation or regulation of the NFkB pathway; and a proliferative disorder associated with abberant NFkB regulation or regulation of the NFkB pathway.

18. A method for treating, or ameliorating a medical condition according to claim 17 wherein the modulator is a member of the group consisting of: a small molecule, a peptide, and an antisense molecule.

19. A method for treating, or ameliorating a medical condition according to claim 18 wherein the modulator is an antagonist.

20. A method for treating, or ameliorating a medical condition according to claim 18 wherein the modulator is an agonist.

21. A method of screening for candidate compounds capable of modulating the activity of a G-protein coupled receptor polypeptide, comprising:
(a) contacting a test compound with a cell or tissue expressing the polypeptide comprising an amino acid sequence as set forth in SEQ ID NO:2; and
(b) selecting as candidate modulating compounds those test compounds that modulate activity of the G-protein coupled receptor polypeptide,
wherein said candidate modulating compounds are useful for the treatment of a disorder.

22. The method according to claim 21 wherein said cells are CHO cells.

23. The method according to claim 22 wherein said cells comprise a vector comprising the coding sequence of the beta lactamase gene under the control of NFAT response elements.

24. The method according to claim 23 wherein said cells further comprise a vector comprising the coding sequence of G alpha 15 under conditions wherein G alpha 15 is expressed.

25. The method according to claim 24 wherein said cells express a member of the group consisting of: the polypeptide of claim 8 at low levels, the polypeptide of claim 8 at moderate levels, the polypeptide of claim 8 at high levels, beta lactamase at low levels, beta lactamase at moderate levels, and beta lactamase at high levels.

26. The method according to claim 25, wherein the disorder is a member of the group consisting of: a reproductive disorder; a male reproductive disorder; a prostate disorder; prostate cancer; proliferative condition of the prostate; cardiovascular disorder; heart disorder; pulmonary disorder; lung disorder; lung cancer; proliferative condition of the lung; gastrointestinal disorder; a colon disorder; colon cancer; female reproductive disorder; ovarian cancer; placental disorder; proliferative condition of the ovary; melanoma; vascular disorders; umbilical cord disorder; disorders associated with aberrant E-selectin expression or activity; disorders associated with aberrant NFkB expression or activity; disorders associated with aberrant IkBalpha expression or activity; an inflammatory disorder; an inflammatory disorder associated with abberant NFkB regulation or regulation of the NFkB pathway; and a proliferative disorder associated with abberant NFkB regulation or regulation of the NFkB pathway.
